(19) United States
(12) Patent Application Publication
(10) Pub. No.: US 2016/0186195 A1
(43) Pub. Date: Jun. 30, 2016

(54) MODIFICATION OF SOYBEAN SEED COMPOSITION TO ENHANCE FEED, FOOD AND OTHER INDUSTRIAL APPLICATIONS OF SOYBEAN PRODUCTS

(71) Applicant: E. I. DU PONT DE NEMOURS AND COMPANY, Wilmington, DE (US)

(72) Inventors: Howard Glenn Damude, Hockessin, DE (US); Knut Meyer, Wilmington, DE (US)

(21) Appl. No.: 14/908,356

(22) PCT Filed: Jul. 30, 2014
(86) PCT No.: PCT/US14/48825

§ 371 Date: Jan. 28, 2016

Related U.S. Application Data
(60) Provisional application No. 61/860,269, filed on Jul. 31, 2013.

(51) Int. Cl. C12N 15/82; A01H 1/02; A01H 5/10

(52) U.S. Cl. CPC C12N 15/8247; A01H 5/10; A01H 1/02; C12N 15/8251; A23L 1/20; A23V 2002/00

(57) ABSTRACT

Polynucleotide sequences encoding diacylglycerol acyltransferases are used in combination with other coding sequence to modify the composition of soybean seed. The modified seed can be used to enhance feed, food and other industrial applications of soybean products.

This application claims the benefit of U.S. Provisional Application No. 61/860,269, filed Jul. 31, 2013, the entire content of which is herein incorporated by reference.

### REFERENCE TO SEQUENCE LISTING SUBMITTED ELECTRONICALLY

The official copy of the sequence listing is submitted electronically via EFS-Web as an ASCII formatted sequence listing with a file named 20140729_BB2124USPSP_SequenceLisitng_ST25 created on Jul. 29, 2014 and having a size of 759 kilobytes and is filed concurrently with the specification. The sequence listing contained in this ASCII formatted document is part of the specification and is herein incorporated by reference in its entirety.

### BACKGROUND OF THE INVENTION

Soybeans are the world's foremost provider of protein and oil representing 30.3 million hectares of crop production in the United Sates in 2011 with a value of over $35.7 billion. Soybeans accounted for 56% of the world oilseed production, with US soybean production accounting for 37% of the world production. Domestically, soybeans provided 66 percent of the edible consumption of fats and oils in the United States. More than 60% of the total value of the US soybean crop was exported as whole soybean, soybean meal or soybean oil.

Soybean oil is used in food products such as margarine, salad dressings and cooking oils, and industrial products such as plastics, biodiesel fuel and transmission fluids. Lecithin is extracted from soybean oil, is used for pharmaceutical applications and protective coatings. After the removal of soybean oil, the remaining flakes can be processed into various edible soy protein products, or used to produce soybean meal for animal feeds. Soy flour and grits are used in the commercial baking industry. Soy hulls are processed into fiber bran breads, cereal and snacks.

The continued dominance of soybean use for the aforementioned applications is dependent upon designing soybean seed compositions that will enhance soybean use and value according to the evolving demands in the food and feed industry.

### SUMMARY OF THE INVENTION

A transgenic soybean seed exhibiting an at least 10% increase in total fatty acids and an at least 1% increase in protein when compared to a control null segregant seed is provided.

In one embodiment, a transgenic soybean seed comprises a recombinant DNA construct which comprises a polynucleotide operably linked to at least one regulatory sequence and encoding one or more of a DGAT polypeptide, an ODP1 polypeptide, and a Lec1 polypeptide. The transgenic soybean seed comprises one or more of a first construct down regulating GAS activity and a second construct down regulating a fad 3 activity, a fad2 activity, or fat2B activity. The transgenic soybean seed exhibits an at least 10% increase in total fatty acids and an at least 1% increase in protein when compared to a null segregant seed. The first construct and the second construct may be on the same construct or on different constructs as the recombinant DNA construct. The regulatory sequence may be a soybean sucrose synthase promoter or a Medicago truncatula sucrose synthase promoter.

Fatty acids may be, but are not limited to palmitic, stearic, oleic, linoleic and linolenic acid.

The ODP1 polypeptide may comprise an amino acid sequence with at least 80%, 85%, 90%, 95%, 96%, 97%, 98%, 99% or 100% identity to SEQ ID NO: 69, SEQ ID NO: 81, or SEQ ID NO:111.

The Lec1 polypeptide may comprise an amino acid sequence with at least 80%, 85%, 90%, 95%, 96%, 97%, 98%, 99% or 100% identity to SEQ ID NO: 83, 94, 99, or 109.

The construct downregulating GAS activity may comprise all or part of nucleotide sequences encoding GAS1, GAS2 or GAS3 polypeptides or any combination thereof, wherein the nucleotide sequences encode amino acid sequences with at least 80%, 85%, 90%, 95%, 96%, 97%, 98%, 99% or 100% identity to SEQ ID NO: 139 (GAS3), SEQ ID NO: 140 (GAS1), or SEQ ID NO:143 (GAS2).

The second construct down regulating a fad 3 activity, a fad2 activity, or a fat2B activity may include one or more nucleotide sequences encoding amino acid sequences having (i) fad 2 activity and with at least 80%, 85%, 90%, 95%, 96%, 97%, 98%, 99% or 100% identity to SEQ ID NO: 119, 121, or 122, (ii) fad 3 activity and with at least 80%, 85%, 90%, 95%, 96%, 97%, 98%, 99% or 100% identity to SEQ ID NO: 129, 131, or 133, and (iii) fatB activity and with at least 80%, 85%, 90%, 95%, 96%, 97%, 98%, 99% or 100% identity to SEQ ID NO: 135 or 137.

In some embodiments, the percent change of palmitic, linoleic and linolenic acid is a decrease when compared to control null segregant seeds. In yet another embodiment, the percent change of oleic acid in the transgenic seed is an increase when compared to a control null segregant seed not comprising the recombinant constructs disclosed herein. The oleic acid can be increased by at least 25% in transgenic seed (s) compared to a control null segregant seed (s). In some embodiments the percent increase in oleic acid is at least 300% when compared to a control seed. In an additional embodiment the percent change of total saturates is a decrease in the transgenic seed compared to control seeds. Additional embodiments include transgenic seed with percent decreases of palmitic, linoleic, linolenic acid, and total saturates and a percent increase of oleic acid when compared to control null segregant seed seeds.

Transgenic soybean seeds may also exhibit a percent decrease in raffinose saccharides compared to null segregant seed. The percent decrease in raffinose saccharides can be at least 60% compared to null segregant seed.

Further embodiments include methods to achieve an increase in total fatty acids and protein content and to alter (increase or decrease) the fatty acid composition of the transgenic seed comprising the constructs described herein compared to null segregant seed. The methods can also include altering the raffinose saccharide, the total saturate, the oleic acid, the palmitic acid, the linoleic acid, and the linolenic acid of the transgenic seeds compared to null segregant seed.

In one embodiment, a method for increasing total fatty acids and protein in a soybean seed comprises the steps of crossing a first transgenic soybean plant with a second transgenic soybean plant to produce a third soybean plant. The first plant in the cross comprises at least one polynucleotide operably linked to at least one regulatory sequence and encodes a DGAT polypeptide, an ODP1 polypeptide, a Lec1 polypeptide or a combination thereof. The second plant in the cross comprises a construct down regulating a fad2 activity. The third soybean plant is selected from the cross and has seed comprising the polynucleotide and the construct, wherein expression of the polypeptide and the construct in the seed results in a % increase in protein in the seed, when compared to the percent increase in protein of a null segregant seed.

The downregulating activity of the construct may be one or more of a fad2, fad3, and fatB activity.

In one embodiment, a method of producing a seed is provided comprising crossing a first transgenic soybean or other species plant with a second transgenic soybean or other species plant. The first plant comprises at least one polynucleotide operably linked to at least one regulatory sequence and encoding a DGAT polypeptide, an ODP1 polypeptide, a Lec1 polypeptide, or a combination thereof. The second plant comprises a construct down regulating a fad2 activity. A third transgenic plant is selected from the crossing and has seed comprising the polynucleotide and the construct and wherein expression of the polynucleotide and the construct results in a percent increase in protein in the seed, when compared to the percent increase of a null segregant seed.

The polypeptide(s) and construct down-regulating activities may be expressed in at least one tissue of the plant, during at least one condition of abiotic stress, or both. The plant may be maize, soybean, sunflower, sorghum, canola, wheat, alfalfa, cotton, rice, barley, millet, sugar cane or switchgrass.

The at least one regulatory sequence can be a sucrose synthase promoter, such as a soybean sucrose synthase promoter or Medicago truncatula sucrose synthase promoter.

The soybean sucrose synthase promoter may comprise a nucleic acid sequence selected from the group consisting of: (a) the nucleic acid sequence of SEQ ID NO: 91; (b) a nucleic acid sequence with at least 95% sequence identity to the nucleic acid sequence of SEQ ID NO: 91; (c) a nucleic acid sequence that hybridizes to SEQ ID NO: 91 under stringent conditions; and (d) a nucleic acid sequence comprising a functional fragment of (a), (b), or (c). The Medicago truncatula sucrose synthase promoter may comprise a nucleic acid sequence selected from the group consisting of: (a) the nucleic acid sequence of SEQ ID NO: 114 or SEQ ID NO: 117; (b) a nucleic acid sequence with at least 95% sequence identity to the nucleic acid sequence of SEQ ID NO: 114 or SEQ ID NO: 117; (c) a nucleic acid sequence that hybridizes to SEQ ID NO: 114 or SEQ ID NO: 117 under stringent conditions; and (d) a nucleic acid sequence comprising a functional fragment of (a), (b) or (c).

Transgenic soybeans produced by the methods disclosed herein are also included.

Any of the transgenic seed described herein may comprise a recombinant construct having at least one DGAT sequence which can be selected from the group consisting of DGAT1, DGAT2 and DGAT1 in combination with DGAT2.

Furthermore, the DGAT sequence can be a Yarrowia sequence or soybean sequence.

The DGAT1 polypeptide may comprise an amino acid sequence with at least 80%, 85%, 90%, 95%. 96%, 97%, 98%, 99% or 100% sequence identity to SEQ ID NO: 105. The DGAT2 polypeptide may comprise an amino acid sequence with at least 80%, 85%, 90%, 95% identity to SEQ ID NO:107. In another embodiment, a plant or a seed comprising any of the recombinant DNA constructs an suppression constructs described above. The plant and the seed may be an oilseed plant and seed. The plant or seed may be a soybean plant or seed.

The percent increase in oil of the transgenic soybean seed may be at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%.

The percent increase in protein of the transgenic soybean seed may be at least 1%, 2%, 3%, 4%, 5%, 6%, 7%, 8%, compared to a non-transgenic soybean. The percent increase in protein of meal obtained from the transgenic soybean seed may be at least 3%, 4,%, 5%, 6%, 7%, 8%, 9%, 10%, 11% or 12% compared to meal obtained from non-transgenic soybean seed.

Any of the transgenic seed described herein may comprise a recombinant construct having downregulated GAS activity.

Also within the scope of the invention are product(s), such as for example meal and/or by-product(s) (e.g. lecithin), and progeny, obtained from the transgenic soybean seeds described herein. Oil and protein products obtained from the transgenic soybean are included, as well as oil and protein products obtained by the methods disclosed herein.

The oil and protein products (such as, for example, meal) can be used as a blending source to make a blended oil or protein product. Blended oil and protein products can be used in the preparation of feed or food.

### BRIEF DESCRIPTION OF THE DRAWINGS

FIG. 1 shows a diagram for the process of production of soybean oils and soybean byproducts.

FIG. 2 shows a schematic of the GmSus promoter region

FIG. 3 shows an alignment comparing the amino acid sequences of Glyma17g00950 (SEQ ID NO: 56), Glyma07g39820 (SEQ ID NO: 59) and GmLec1 (SEQ ID NO: 64).

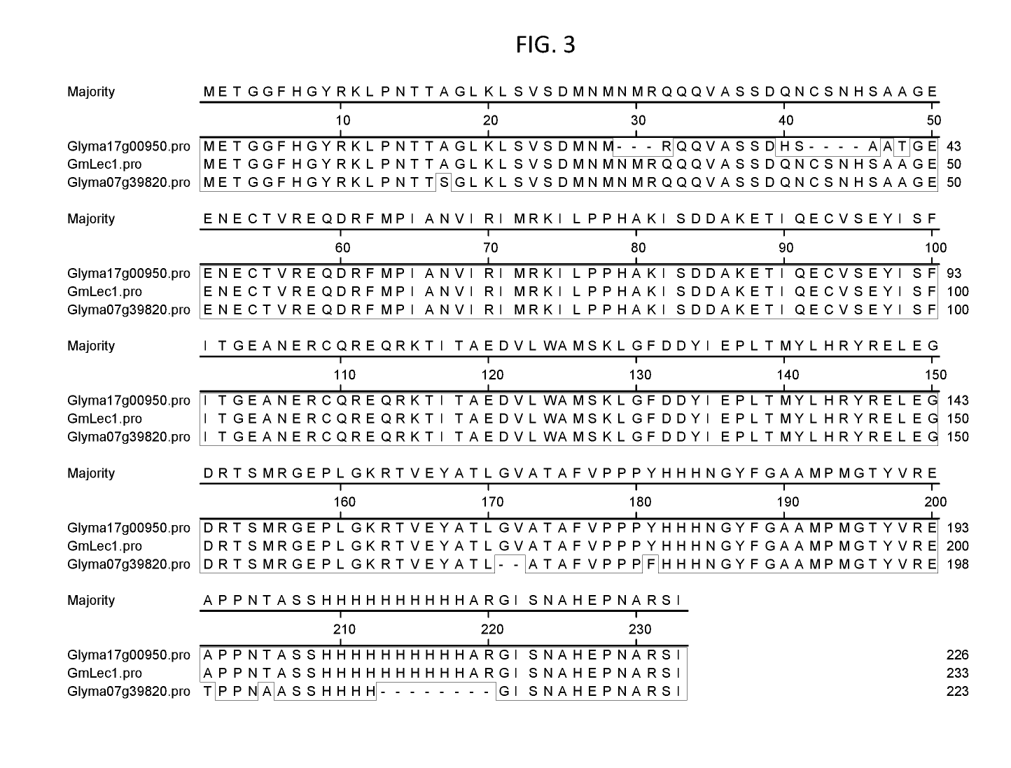

### SEQUENCE LISTINGS

The sequence descriptions summarize the Sequences Listing attached hereto. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).
SEQ ID NO: 1 corresponds to the nucleotide sequence of plasmid pKR1756.
SEQ ID NO: 2 corresponds to the nucleotide sequence of 159-fad3c amiRNA
SEQ ID NO: 3 corresponds to the nucleotide sequence of the Ann-fad3c-BD30 BsiWI/SbfI fragment.
SEQ ID NO: 4 corresponds to the nucleotide sequence of plasmid pKR277.
SEQ ID NO: 5 corresponds to the nucleotide sequence of plasmid pKR1850.
SEQ ID NO: 6 corresponds to the nucleotide sequence of plasmid KS362.
SEQ ID NO: 7 corresponds to the nucleotide sequence of the BsiWI fragment containing the beta conglycin/YLDGAT2/phaseolin cassette. SEQ ID NO: 8 corresponds to the nucleotide sequence of plasmid pKR1975. SEQ ID NO: 9 corresponds to the nucleotide sequence of the donor construct QC632.
SEQ ID NO: 10 corresponds to the nucleotide sequence of the PINII terminator.
SEQ ID NO: 11 corresponds to the nucleotide sequence of plasmid pKR1763.
SEQ ID NO: 12 corresponds to the nucleotide sequence of the DNA fragment of the 3′ transcription terminator region of the phaseolin gene with flanking ORFstop sequences (ORFstopA and ORFstopB as well as flanking BsiWi/MluI sites.
SEQ ID NO: 13 corresponds to the nucleotide sequence of plasmid pKR1849.
SEQ ID NO: 14 corresponds to the nucleotide sequence of plasmid pKR1857.
SEQ ID NO: 15 corresponds to the nucleotide sequence of plasmid pKR1980.
SEQ ID NO: 16 corresponds to the nucleotide sequence of the Not1 fragment containing Gas123 hp.
SEQ ID NO: 17 corresponds to the nucleotide sequence of plasmid pKR1273.
SEQ ID NO: 18 corresponds to the nucleotide sequence of plasmid pKR1292.
SEQ ID NO: 19 corresponds to the nucleotide sequence of plasmid pKR1986 (PHP50573).
SEQ ID NO: 20 corresponds to the nucleotide sequence of the expression plasmid QC292.
SEQ ID NO: 21 corresponds to the nucleotide sequence of plasmid QC608 (PHP44664).
SEQ ID NO: 22 corresponds to the nucleotide sequence of the recombination product of the frt1 and frt87 sites from Target line A with those in plasmid PHP70573.
SEQ ID NO: 23 corresponds to the nucleotide sequence of plasmid pKR1776.
SEQ ID NO: 24 corresponds to the nucleotide sequence of plasmid pKR1896.
SEQ ID NO: 25 corresponds to the nucleotide sequence of plasmid KS362.
SEQ ID NO: 26 corresponds to the nucleotide sequence of plasmid pKR264.
SEQ ID NO: 27 corresponds to the nucleotide sequence of plasmid pKR1972.
SEQ ID NO: 28 corresponds to the nucleotide sequence of plasmid pKR2085.
SEQ ID NO: 29 corresponds to the nucleotide sequence of plasmid pKR2008.
SEQ ID NO: 30 corresponds to the nucleotide sequence of plasmid KR2087.
SEQ ID NO: 31 corresponds to the nucleotide sequence of plasmid pKR2101 (PHP52246).
SEQ ID NO: 32 corresponds to the nucleotide sequence of plasmid pLF179.
SEQ ID NO: 33 corresponds to the nucleotide sequence of plasmid pKR1995.
SEQ ID NO: 34 corresponds to the nucleotide sequence of plasmid pKR2086.
SEQ ID NO: 35 corresponds to the nucleotide sequence of plasmid pKR2088.
SEQ ID NO: 36 corresponds to the nucleotide sequence of plasmid pKR2102 (PHP52247).
SEQ ID NO: 37 corresponds to the nucleotide sequence of recombination product frt1 and frt87 sites from Target line A with those in plasmid PHP52246.
SEQ ID NO: 38 corresponds to the nucleotide sequence of recombination product frt1 and frt87 sites from Target line A with those in plasmid PHP52247.
SEQ ID NO: 39 corresponds to the nucleotide sequence of the Arabidopsis Sucrose Synthase 2 gene.
SEQ ID NO: 40 corresponds to the amino acid sequence of the Arabidopsis Sucrose Synthase 2 gene.
SEQ ID NO: 41 corresponds to the nucleotide sequence of the predicted genomic soybean homolog of the Arabidopsis Sucrose Synthase 2 gene.
SEQ ID NO: 42 corresponds to the nucleotide sequence of the cDNA of the soybean homolog to the Arabidopsis Sucrose Synthase 2.
SEQ ID NO: 43 corresponds to the CDS of the soybean homolog to the Arabidopsis Sucrose Synthase 2.
SEQ ID NO: 44 corresponds to the amino acid sequence of the soybean homolog to the Arabidopsis Sucrose Synthase 2.
SEQ ID NO: 45 corresponds to the sequence for the 5′ end of EST sdp3c.pk014.n18.
SEQ ID NO: 46 corresponds to the sequence of the promoter region of the soybean homolog to the Arabidopsis Sucrose Synthase 2 (GmSus promoter region).
SEQ ID NO: 47 corresponds to the sequence AW box.
SEQ ID NO: 48 corresponds to theGmSuSYProm-5 oligonucleotide sequence (forward primer).
SEQ ID NO: 49 corresponds to the GmSuSYProm-5 oligonucleotide sequence (reverse primer).
SEQ ID NO: 50 corresponds to the nucleotide sequence of plasmid pLF284.
SEQ ID NO: 51 corresponds to the nucleotide sequence of plasmid pKR1963.
SEQ ID NO: 52 corresponds to the nucleotide sequence of plasmid pKR1964.
SEQ ID NO: 53 corresponds to the nucleotide sequence of plasmid pKR1965.
SEQ ID NO: 54 corresponds to the nucleotide sequence of cDNA clone se2.11d12.
SEQ ID NO: 55 corresponds to the coding sequence from clone se2.11d12-Glyma17g00950.
SEQ ID NO: 56 corresponds to the amino acid sequence of se2.11d12-Glyma17g00950.
SEQ ID NO: 57 corresponds to the full insert sequence of se1.pk0042.d8.
SEQ ID NO: 58 corresponds to the coding sequence of clone se1.pk0042.d8.
SEQ ID NO: 59 corresponds to the amino acid sequence of clone se1.pk0042.d8.
SEQ ID NO: 60 corresponds to the oligonucleotide sequence SA275.
SEQ ID NO: 61 corresponds to the oligonucleotide sequence SA276.
SEQ ID NO: 62 corresponds to the nucleotide sequence of plasmid Glyma17g00950/pCR8/GW/TOPO.
SEQ ID NO: 63 corresponds to the CDS from the PCR product contained in Glyma17g00950/pCR8/GW/TOPO, named GmLec1.
SEQ ID NO: 64 corresponds to the amino acid sequence of GmLec1.
SEQ ID NO: 65 corresponds to the oligonucleotide sequence Gmlec-5.
SEQ ID NO: 66 corresponds to the oligonucleotide sequence Gmlec-3.
SEQ ID NO: 67 corresponds to the nucleotide sequence of plasmid pLF275.
SEQ ID NO: 68 corresponds to the sequence of CDS GmODP1.
SEQ ID NO: 69 corresponds to the amino acid sequence of GmODP1.
SEQ ID NO: 70 corresponds to the 396b-GM-MFAD2-1B STAR sequence.
SEQ ID NO: 71 corresponds to the 159-GM-MFAD2-2 STAR sequence.
SEQ ID NO: 72 corresponds to the genomic miRNA precursor sequence 159.
SEQ ID NO: 73 corresponds to the genomic miRNA precursor sequence 396b.
SEQ ID NO: 74 corresponds to the miRNA precursor sequence 396b-fad2-1b/159-fad2-2.
SEQ ID NO: 75 corresponds to the sequence of soybean expression vector pKR2109.
SEQ ID NO: 76 corresponds to the nucleotide sequence of plasmid pKR1968.
SEQ ID NO: 77 corresponds to the nucleotide sequence of plasmid pKR1971.
SEQ ID NO: 78 corresponds to the nucleotide sequence of plasmid pKR2118.
SEQ ID NO: 79 corresponds to the nucleotide sequence of plasmid pKR2120.
SEQ ID NO: 80 corresponds to the GmODP1 nucleotide sequence.
SEQ ID NO: 81 corresponds to the GmODP1 amino acid sequence.
SEQ ID NO: 82 corresponds to the GmLec1 nucleotide sequence.
SEQ ID NO: 83 corresponds to the GmzLec1 amino acid sequence.
SEQ ID NO:84 corresponds to the sequence of GM-MFAD2-1B.
SEQ ID NO:85 corresponds to the sequence of GM-MFAD2-2.
SEQ ID NO: 86 is the genomic sequence of the soybean Sucrose Synthase gene corresponding to the locus Glyma13g17420.
SEQ ID NO: 87 is the cDNA sequence of the soybean Sucrose Synthase gene corresponding to the locus Glyma13g17420.
SEQ ID NO: 88 is the CDS (coding sequence) of the soybean Sucrose Synthase gene corresponding to the locus Glyma13g17420. The soybean homolog to the Arabidopsis sucrose synthase 2 gene set forth in SEQ ID NO: 5 is called GmSuS.
SEQ ID NO: 89 is the amino acid sequence encoded by SEQ ID NO: 5, and is the sequence of soybean Sucrose Synthase polypeptide.
SEQ ID NO: 90 is the sequence for the 5′ end of EST sdp3c.pk014.n18.
SEQ ID NO: 91 is the sequence of the genomic DNA upstream of the start codon of GmSuS (SEQ ID NO: 5), corresponding to the promoter for GmSuS.
SEQ ID NO: 92 is the sequence of the cDNA clone se2.11d12.
SEQ ID NO: 93 is the sequence of the soybean clone se2.11d12 from 38-718 bp, and is the coding sequence of Lec1b (GI: 158525282) and corresponds to Glyma17g00950.
SEQ ID NO: 94 is the amino acid sequence encoded by the nucleotide sequence given in SEQ ID NO: 16.
SEQ ID NO: 95 is the full insert sequence of the cDNA clone se1.pk0042.d8.
SEQ ID NO: 96 is the sequence from soybean cDNA clone se1.pk0042.d8 with a corrected start site, corresponding to Glyma07g39820.
SEQ ID NO: 97 is the amino acid sequence encoded by the sequence given in SEQ ID NO: 96.
SEQ ID NO: 98 is the nucleotide sequence of GmLec1.
SEQ ID NO: 99 is the amino acid sequence encoded by the nucleotide sequence given in SEQ ID NO: 98.
SEQ ID NO: 100 is the CDS of GmODP1.
SEQ ID NO: 101 is the amino acid sequence of GmODP1.
SEQ ID NO: 102 is the predicted CDS for Glyma16g05480.
SEQ ID NO: 103 is the amino acid sequence for Glyma16g05480.
SEQ ID NO: 104 is the CDS of GmDGAT1cAII.
SEQ ID NO: 105 is the amino acid sequence of GmDGAT1cAII.
SEQ ID NO: 106 is the CDS of YLDGAT2.
SEQ ID NO: 107 is the amino acid sequence of YLDGAT2.
SEQ ID NO: 108 is the CDS of ZmLec1.
SEQ ID NO: 109 is the amino acid sequence of ZmLec1.
SEQ ID NO: 110 is the CDS of ZmODP1.
SEQ ID NO: 111 is the amino acid sequence of ZmODP1.
SEQ ID NO: 112 is a conserved Lec1 sequence motif.
SEQ ID NO: 113 is the nucleotide sequence of the AW box.
SEQ ID NO: 114 is the nucleotide sequence of the predicted CDS for Medtr4g124660.2.
SEQ ID NO: 115 is the amino acid sequence encoded by SEQ ID NO: 79.
SEQ ID NO: 116 is the predicted nucleotide sequence of the Medtr4g124660.2 promoter region.
SEQ ID NO: 117 is the actual nucleotide sequence of the Medtr4g124660.2 promoter region used.
SEQ ID NO:118 corresponds to the nucleotide sequence of soy fatty acid biosynthetic gene Glyma10g42470 (GmFad2-1) targeted for silencing.
SEQ ID NO:119 corresponds to the amino acid sequence encoded by SEQ ID NO:118.
SEQ ID NO:120 corresponds to the nucleotide sequence of soy fatty acid biosynthetic gene Glyma20g24530 (GmFad2-1) targeted for silencing.
SEQ ID NO:121 corresponds to the amino acid sequence encoded by SEQ ID NO:120.
SEQ ID NO:122 corresponds to the nucleotide sequence of soy fatty acid biosynthetic gene Glyma19g32940 (Fad2-2) targeted for silencing.
SEQ ID NO:123 corresponds to the amino acid sequence encoded by SEQ ID NO:122.
SEQ ID NO:124 corresponds to the nucleotide sequence of soy fatty acid biosynthetic gene Glyma02g15600 (GmSad) targeted for silencing.
SEQ ID NO:125 corresponds to the amino acid sequence encoded by SEQ ID NO:124.
SEQ ID NO:126 corresponds to the nucleotide sequence of soy fatty acid biosynthetic gene Glyma07g32850 (GmSad) targeted for silencing.
SEQ ID NO:127 corresponds to the amino acid sequence encoded by SEQ ID NO:126.
SEQ ID NO: 128 corresponds to the nucleotide sequence of soy fatty acid biosynthetic gene Glyma14g37350 (GmFad3) targeted for silencing.
SEQ ID NO:129 corresponds to the amino acid sequence encoded by SEQ ID NO:128.
SEQ ID NO:130 corresponds to the nucleotide sequence of soy fatty acid biosynthetic gene Glyma02g39230 (GmFad3) targeted for silencing.
SEQ ID NO:131 corresponds to the amino acid sequence encoded by SEQ ID NO:130.
SEQ ID NO:132 corresponds to the nucleotide sequence of soy fatty acid biosynthetic gene Glyma18g06950 (GmFad3) targeted for silencing.
SEQ ID NO:133 corresponds to the amino acid sequence encoded by SEQ ID NO:132.
SEQ ID NO:134 corresponds to the nucleotide sequence of soy fatty acid biosynthetic gene Glyma05g08060 (FatB) targeted for silencing.
SEQ ID NO:135 corresponds to the amino acid sequence encoded by SEQ ID NO:134.
SEQ ID NO:136 corresponds to the nucleotide sequence of soy fatty acid biosynthetic gene Glyma17g12940 (FatB) targeted for silencing.
SEQ ID NO:137 corresponds to the amino acid sequence encoded by SEQ ID NO:136.
SEQ ID NO:138 is the 1151 by sequence derived from clone sdp3c.pk013.c9 (FIS) of the soybean nucleotide sequence containing the ORF [nucleotides 71-1090 (Stop)] of the galactinol synthase 3 gene.
SEQ ID NO:139 is the 339 amino acid sequence encoded by the ORF [nucleotides 71-1090 (Stop)] of SEQ ID NO: 138.
SEQ ID NO:140 represents the DNA sequence of the soybean galactinol synthase gene GAS1.
SEQ ID NO:141 represents the putative translation product DNA sequence of SEQ ID NO:140 the soybean galactinol synthase gene GAS1.
SEQ ID NO:142 represents the DNA sequence of the soybean galactinol synthase gene GAS2.
SEQ ID NO:143 represents the putative translation product DNA sequence of SEQ ID NO:142 the soybean galactinol synthase gene GAS2.

### DETAILED DESCRIPTION OF THE INVENTION

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

In the context of this disclosure, a number of terms and abbreviations are used as follows ALS (acetolactate synthase protein), by (base pair), FAD2 (microsomal omega-6 desaturase protein), gm-fad2-1 (soybean microsomal omega-6 desaturase gene 1), gm-als (wild type acetolactate synthase gene from soybean), gm-hra (modified version of acetolactate synthase gene from soybean), kb (kilobase), PCR (polymerase chain reaction) and UTR (untranslated region).

“microRNA or miRNA” refers to oligoribonucleic acid which regulates expression of a polynucleotide comprising the target sequence. microRNAs (miRNAs) are noncoding RNAs of about 19 to about 24 nucleotides (nt) in length that have been identified in both animals and which regulate expression of a polynucleotide comprising the target sequence. They are processed from longer precursor transcripts that range in size from approximately 70 to 2000 nt or longer, and these precursor transcripts have the ability to form stable hairpin structures.

“pri-miRNAs” or “primary miRNAs” are long, polyadenylated RNAs transcribed by RNA polymerase II that encode miRNAs. “pre-miRNAs” are primary miRNAs that have been processed to form a shorter sequence that has the capacity to form a stable hairpin and is further processed to release a miRNA. In plants both processing steps are carried out by dicerlike and it is therefore difficult to functionally differentiate between “pri-miRNAs” and “pre-miRNAs”. Therefore, a precursor miRNA, or a primary miRNA, is functionally defined herein as a nucleotide sequence that is capable of producing a miRNA. Given this functional definition, and as will be clear from the Examples and discussion herein, a precursor miRNA, primary miRNA and/or a miRNA can be represented as a ribonucleic acid or, alternatively, in a deoxyribonucleic acid form that “corresponds substantially” to the precursor miRNA, primary miRNA and/or miRNA. It is understood that the DNA in its double-stranded form will comprise a strand capable of being transcribed into the miRNA precursor described. Expression constructs, recombinant DNA constructs, and transgenic organisms incorporating the miRNA encoding DNA that results in the expression of the described miRNA precursors are described.

A “variable nucleotide subsequence” refers to a portion of a nucleotide sequence that replaces a portion of a pre-miRNA sequence provided that this subsequence is different from the sequence that is being replaced, i.e., it cannot be the same sequence.

A “target gene” refers to a gene that encodes a target RNA, i.e., a gene from which a target RNA is transcribed. The gene may encode mRNA, tRNA, small RNA, etc.

A “target sequence” refers to an RNA whose expression is to be modulated, e.g., down-regulated. The target sequence may be a portion of an open reading frame, 5′ or 3′ untranslated region, exon(s), intron(s), flanking region, etc.

A “star sequence” is the complementary sequence within a miRNA precursor that forms a duplex with the miRNA. The complementarity of the star sequence does not need to be perfect. Non-helix disrupting substitutions (i.e. G:T base pairs etc.) are sometimes found, as well as 1-3 mismatches.

In the context of this disclosure, a number of terms and abbreviations are used. The following definitions are provided.

The term “percentage points” (pp) refers to the arithmetic difference of two percentages, e.g. [transgenic value (%)−control value (%)]=percentage points. The term “relative change”, “percent change”, “percent increase”, or “percent decrease” refers to a change expressed as a fraction of the control value, e.g. {[transgenic value(%)−control value (%)]/control value (%)}×100%=percent change.

The control is a seed, plant, plant part or product comparable to the so transgenic seed, plant, plant part or product which, unless specified to the contrary, lacks the transgenes or is obtained from material lacking the transgenes. In certain embodiments, the control lacks constructs which downregulate specified activities, but which includes the DGAT, OPD1 or Lec1 encoding polynucleotide. In certain embodiments, the control lacks both the constructs downregulating specified activities and the DGAT, OPD1 or Lec1 encoding polynucleotide. In certain embodiments the control includes a fad 2-downregulating construct, but lacks DGAT encoding polynucleotide. In certain embodiments the control is a non-transgenic, null segregant soybean plant, plant part or seed.

“Non-transgenic, null segregant soybean, control null segregant” refers to a control near isogenic plant, plant part or seed that lacks the transgene (unless otherwise stated), and/or a control parental plant used in the transformation process to obtain the transgenic event. Null segregants can be plants, plant parts or seed that do not contain the transgenic trait due to normal genetic segregation during propagation of the heterozygous transgenic plants.

The term “or combinations thereof” as used herein refers to all permutations and combinations of the listed items preceding the term. For example, “A, B, C, or combinations thereof’ is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

“Open reading frame” is abbreviated ORF.

“Polymerase chain reaction” is abbreviated PCR.

“American Type Culture Collection” is abbreviated ATCC.

Acyl-CoA:sterol-acyltransferase” is abbreviated ARE2.

“Phospholipid:diacylglycerol acyltransferase” is abbreviated PDAT.

“Diacylglycerol acyltransferase” is abbreviated DAG AT or DGAT.

“Diacylglycerol” is abbreviated DAG.

“Triacylglycerols” are abbreviated TAGs.

“Co-enzyme A” is abbreviated CoA.

“Plastidic Phosphoglucomutase” is abbreviated PGM.

“Galactinol Synthase” is abbreviated GAS.

FatB is a thioesterase encoding a palmitoyl-thioesterase (Kinney, A. J. (1997) Genetic engineering of oilseeds for desired traits. In: Genetic Engineering, Vol. 19, (Setlow J. K. Plenum Press, New York, N.Y., pp. 149-166.).

The term “ODP1” refers to an ovule development protein 1 that is involved with increasing oil content. ODP1 is a member of the APETALA2 (AP2) family of proteins that play a role in a variety of biological events including, but not limited to, oil content.

U.S. Pat. No. 8,404,926 describes the use of an ODP1 gene for alteration of oil traits in plants. U.S. Pat. No. 7,579,529 describes an AP2 domain transcription factor and methods of its use. U.S. Pat. No. 7,157,621 discloses the use of ODP1 transcription factor for increasing oil content in plants. International patent application WO 2010/114989 describes the use of an Arabidopsis Sus2 promoter to drive ODP1 (WRI1) expression in Arabidopsis. The disclosures of each of these patents and publications are herein incorporated by reference in their entireties.

Leafy cotyledon1 (Lec1 or Lec1/Hap3) is a transcription factor that is a key regulator of seed development in plants. Lec1 is a CCAAT-binding factor (CBF)—type transcription factor. The terms “leafy cotyledon 1”, “Lec1”, and “Hap3/Lec1” are used interchangeably herein. LEC1 polypeptide is homologous to the HAP3 subunit of the CBF class of eukaryotic transcriptional activators that includes NF-Y, CP1, and HAP2/3/4/5 (Lotan et al. (1998) Cell, Vol. 93, 1195-1205, June 26).

The leafy cotyledon1 (LEC1) gene controls many distinct aspects of embryogenesis. The lec1 mutation is pleiotropic, which suggest that LEC1 has several roles in late embryo development. For example, LEC1 is required for specific aspects of seed maturation, inhibiting premature germination and plays a role in the specification of embryonic organ identity. Finally, LEC1 appears to act only during embryo development.

U.S. Pat. No. 6,235,975 describes leafy cotyledon1 genes and their uses. U.S. Pat. No. 7,888,560 relates to isolated nucleic acid fragments encoding Led related transcription factors. U.S. Pat. Nos. 7,294,759, 7,157,621, 7,888,560, and 6,825,397 describe the use of Lec1 genes for altering oil content in plants. The disclosures of each of these patents are herein incorporated by reference in their entireties.

In Arabidopsis, Lec1 has been shown to regulate the expression of fatty acid biosynthetic genes and Lec1 has also been shown to be involved in embryo development (Mu et al., Plant Physiology (2008) 148: 1042-1054; Lotan et al. (1998) Cell, Vol. 93, 1195-1205, June 26; PCT publication number WO/1998037184 & U.S. Pat. Nos. 6,235,975, 6,320,102, 6,545,201; PCT publication no. WO/2001064022 & U.S. Pat. No. 6,781,035, Braybrook, S. A. and Harada, J. J. (2008) Trends Plant Sci 13(12): 1360-1385). The disclosures of each of these patents and applications are herein incorporated by reference in their entireties.

WO 99/67405 describes leafy cotyledon1 genes and their uses. A maize Lec1 homologue of the Arabidopsis embryogenesis controlling gene AtLEC1 has been shown to increase oil content and transformation efficiencies in plants. See, for example, WO 03001902 and U.S. Pat. No. 6,512,165. The disclosures of each of these patents and applications are herein incorporated by reference in their entireties.

Other polypeptides that influence ovule and embryo development and stimulate cell growth, such as, Led, Kn1, WUSCHEL, Zwille and Aintegumeta (ANT) allow for increased transformation efficiencies when expressed in plants. See, for example, U.S. Application No. 2003/0135889, herein incorporated by reference. In fact, a maize Lec1 homologue of the Arabidopsis embryogenesis controlling gene AtLEC1, has been shown to increase oil content and transformation efficiencies in plants. See, for example, WO 03001902 and U.S. Pat. No. 6,512,165. The disclosures of each of these patents and applications are herein incorporated by reference in their entireties.

The term “fatty acids” refers to long chain aliphatic acids (alkanoic acids) of varying chain length, from about C₁₂ to C₂₂ (although both longer and shorter chain-length acids are known). The predominant chain lengths are between C₁₆ and C₂₂. The structure of a fatty acid is represented by a simple notation system of “X:Y”, so where X is the total number of carbon (C) atoms in the particular fatty acid and Y is the number of double bonds.

Generally, fatty acids are classified as saturated or unsaturated. The term “saturated fatty acids” refers to those fatty acids that have no “double bonds” between their carbon backbone. In contrast, “unsaturated fatty acids” have “double bonds” along their carbon backbones (which are most commonly in the cis-configuration). “Monounsaturated fatty acids” have only one “double bond” along the carbon backbone (e.g., usually between the 9^{th} and 10^{th} carbon atom as for palmitoleic acid (16:1) and oleic acid (18:1)), while “polyunsaturated fatty acids” (or “PUFAs”) have at least two double bonds along the carbon backbone (e.g., between the 9^{th} and 10^{th}, and 12^{th} and 13^{th} carbon atoms for linoleic acid (18:2); and between the 9^{th} and 10^{th}, 12^{th} and 13^{th}, and 15^{th} and 16^{th} for α-linolenic acid (18:3)).

“Microbial oils” or “single cell oils” are those oils naturally produced by microorganisms (e.g., algae, oleaginous yeasts and filamentous fungi) during their lifespan. The term “oil” refers to a lipid substance that is liquid at 25° C. and usually polyunsaturated. In contrast, the term “fat” refers to a lipid substance that is solid at 25° C. and usually saturated.

“Lipid bodies” refer to lipid droplets that usually are bounded by specific proteins and a monolayer of phospholipid. These organelles are sites where most organisms transport/store neutral lipids. Lipid bodies are thought to arise from microdomains of the endoplasmic reticulum that contain TAG-biosynthesis enzymes; and, their synthesis and size appear to be controlled by specific protein components.

“Neutral lipids” refer to those lipids commonly found in cells in lipid bodies as storage fats and oils and are so called because at cellular pH, the lipids bear no charged groups. Generally, they are completely non-polar with no affinity for water. Neutral lipids generally refer to mono-, di-, and/or triesters of glycerol with fatty acids, also called monoacylglycerol, diacylglycerol or TAG, respectively (or collectively, acylglycerols). A hydrolysis reaction must occur to release free fatty acids from acylglycerols.

The terms “triacylglycerol”, “oil” and “TAGs” refer to neutral lipids composed of three fatty acyl residues esterified to a glycerol molecule (and such terms will be so used interchangeably throughout the present disclosure herein). Such oils can contain long chain PUFAs, as well as shorter saturated and unsaturated fatty acids and longer chain saturated fatty acids. Thus, “oil biosynthesis” generically refers to the synthesis of TAGs in the cell.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores.

“Progeny” comprises any subsequent generation of a plant.

The terms “monocot” and “monocotyledonous plant” are used interchangeably herein. Monocots include the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without so limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Progeny” comprises any subsequent generation of a plant.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

An “Expressed Sequence Tag” (“EST”) is a DNA sequence derived from a cDNA library and therefore is a sequence which has been transcribed. An EST is typically obtained by a single sequencing pass of a cDNA insert.

“Mature” protein refers to a post-translationally processed polypeptide; i.e., one from which any pre- or pro-peptides present in the primary translation product has been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment.

Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The terms “full complement” and “full-length complement” are used interchangeably herein, and refer to a complement of a given nucleotide sequence, wherein the complement and the nucleotide sequence consist of the same number of nucleotides and are 100% complementary.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

The terms “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such a construct may be used by itself or may be used in conjunction with a vector.

This construct may comprise any combination of deoxyribonucleotides, ribonucleotides, and/or modified nucleotides. The construct may be transcribed to form an RNA, wherein the RNA may be capable of forming a double-stranded RNA and/or hairpin structure. This construct may be expressed in the cell, or isolated or synthetically produced. The construct may further comprise a promoter, or other sequences which facilitate manipulation or expression of the construct.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential in the structure, the stability, or the activity of a protein.

Because they are identified by their high degree of conservation in aligned sequences of a family of protein homologues, they can be used as identifiers, or “signatures”, to determine if a protein with a newly determined sequence belongs to a previously identified protein family.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

“Tissue-specific promoter” and “tissue-preferred promoter” are used interchangeably to refer to a promoter that is expressed predominantly but not necessarily exclusively in one tissue or organ, but that may also be expressed in one specific cell. Examples of some seed-specific promoters are the alpha prime subunit of beta conglycinin promoter, soybean sucrose synthase promoter, Medicago trunculatis sucrose synthase promoter, Kunitz trypsin inhibitor 3, annexin promoter, Glyl promoter, beta subunit of beta conglycinin promoter, P34/Gly Bd m 30K promoter, albumin promoter, Leg A1 promoter and Leg A2 promoter.

“Developmentally regulated promoter” refers to a promoter whose activity is determined by developmental events.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Examples of inducible or so regulated promoters include, but are not limited to, promoters regulated by light, heat, stress, flooding or drought, pathogens, phytohormones, wounding, or chemicals such as ethanol, jasmonate, salicylic acid, or safeners.

A minimal or basal promoter is a polynucleotide molecule that is capable of recruiting and binding the basal transcription machinery. One example of basal transcription machinery in eukaryotic cells is the RNA polymerase II complex and its accessory proteins.

Plant RNA polymerase II promoters, like those of other higher eukaryotes, are comprised of several distinct “cis-acting transcriptional regulatory elements,” or simply “cis-elements,” each of which appears to confer a different aspect of the overall control of gene expression. Examples of such cis-acting elements include, but are not limited to, such as TATA box and CCAAT or AGGA box. The promoter can roughly be divided in two parts: a proximal part, referred to as the core, and a distal part. The proximal part is believed to be responsible for correctly assembling the RNA polymerase II complex at the right position and for directing a basal level of transcription, and is also referred to as “minimal promoter” or “basal promoter”. The distal part of the promoter is believed to contain those elements that regulate the spatio-temporal expression. In addition to the proximal and distal parts, other regulatory regions have also been described, that contain enhancer and/or repressors elements The latter elements can be found from a few kilobase pairs upstream from the transcription start site, in the introns, or even at the 3′ side of the genes they regulate (Rombauts, S. et al. (2003) Plant Physiology 132:1162-1176, Nikolov and Burley, (1997) Proc Natl Acad Sci USA 94: 15-22), Tjian and Maniatis (1994) Cell 77: 5-8; Fessele et al., 2002 Trends Genet 18: 60-63, Messing et al., (1983) Genetic Engineering of Plants: an Agricultural Perspective, Plenum Press, NY, pp 211-227).

When operably linked to a heterologous polynucleotide sequence, a promoter controls the transcription of the linked polynucleotide sequence.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Overexpression” refers to the production of a gene product in transgenic organisms that exceeds levels of production in a control.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as sRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065, incorporated herein by reference). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

Such a recombinant construct promoter would comprise different components such as a promoter which is a DNA sequence that directs cellular machinery of a plant to produce RNA from the contiguous coding sequence downstream (3′) of the promoter. The promoter region influences the rate, developmental stage, and cell type in which the RNA transcript of the gene is made. The RNA transcript is processed to produce mRNA which serves as a template for translation of the RNA sequence into the amino acid sequence of the encoded polypeptide. The 5′ non-translated leader sequence is a region of the mRNA upstream of the protein coding region that may play a role in initiation and translation of the mRNA. The 3′ transcription termination/polyadenylation signal is a non-translated region downstream of the protein coding region that functions in the plant cell to cause termination of the RNA transcript and the addition of polyadenylate nucleotides to the 3′ end of the RNA.

The origin of the promoter chosen to drive expression of the coding sequences of the polynucleotides disclosed herein is not important as long as it has sufficient transcriptional activity to express translatable mRNA for the desired nucleic acid fragments in the desired host tissue at the right time. Either heterologous or non-heterologous (i.e., endogenous) promoters can be used to in the methods and compositions. For example, suitable promoters include, but are not limited to: the alpha prime subunit of beta conglycinin promoter, the Kunitz trypsin inhibitor 3 promoter, the annexin promoter, the glycinin Gy1 promoter, the beta subunit of beta conglycinin promoter, the P34/Gly Bd m 30K promoter, the albumin promoter, the Leg A1 promoter and the Leg A2 promoter.

The annexin, or P34, promoter is described in PCT Publication No. WO 2004/071178 (published Aug. 26, 2004). The level of activity of the annexin promoter is comparable to that of many known strong promoters, such as: (1) the CaMV 35S promoter (Atanassova et al., Plant Mol. Biol. 37:275-285 (1998); Battraw and Hall, Plant Mol. Biol. 15:527-538 (1990); Holtorf et al., Plant Mol. 29:637-646 (1995); Jefferson et al., EMBO J. 6:3901-3907 (1987); Wilmink et al., Plant Mol. Biol. 28:949-955 (1995)); (2) the Arabidopsis oleosin promoters (Plant et al., Plant Mol. Biol. 25:193-205 (1994); Li, Texas A & M University Ph.D. dissertation, pp. 107-128 (1997)); (3) the Arabidopsis ubiquitin extension protein promoters (Callis et al., J Biol. Chem. 265(21):12486-93 (1990)); (4) a tomato ubiquitin gene promoter (Rollfinke et al., Gene. 211(2):267-76 (1998)); (5) a soybean heat shock protein promoter (Schoffl et al., Mol Gen Genet. 217(2-3):246-53 (1989)); and, (6) a maize H3 histone gene promoter (Atanassova et al., Plant Mol Biol. 37(2):275-85 (1989)).

Another useful feature of the annexin promoter is its expression profile in developing seeds. The annexin promoter is most active in developing seeds at early stages (before 10 days after pollination) and is largely quiescent in later stages. The expression profile of the annexin promoter is different from that of many seed-specific promoters, e.g., seed storage protein promoters, which often provide highest activity in later stages of development (Chen et al., Dev. Genet. 10:112-122 (1989); Ellerstrom et al., Plant Mol. Biol. 32:1019-1027 (1996); Keddie et al., Plant Mol. Biol. 24:327-340 (1994); Plant et al., (supra); Li, (supra)). The annexin promoter has a more conventional expression profile but remains distinct from other known seed specific promoters. Thus, the annexin promoter will be a very attractive candidate when overexpression, or suppression, of a gene in embryos is desired at an early developing stage. For example, it may be desirable to overexpress a gene regulating early embryo development or a gene involved in the metabolism prior to seed maturation.

Following identification of an appropriate promoter suitable for expression of a specific coding sequence of the polynucleotides described herein, the promoter is then operably linked in a sense orientation using conventional means well known to those skilled in the art.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N. Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

In one embodiment a transgenic soybean seed comprising a recombinant DNA construct, the recombinant DNA construct comprising at least one polynucleotide encoding a polypeptide selected from the group consisting of (i) a DGAT polypeptide, (ii) an ODP1 polypeptide, (iii) a Lec1 polypeptide, and (iv) a combination thereof, the polynucleotide being linked to at least one regulatory sequence, and wherein the transgenic soybean seed comprises one or more of (i) a first construct down regulating GAS activity, and (ii) a second construct down regulating a fad 3 activity, a fad2 activity, or fat2B activity, wherein the transgenic soybean seed exhibits a percent increase in total fatty acid of at least 10%, and a percent increase in protein of at least 1%, when compared to a control null segregant seed. The first construct and the second construct may be on the same construct or on different constructs as the recombinant DNA construct. The regulatory sequence may be a soybean sucrose synthase promoter or a Medicago truncatula sucrose synthase promoter.

Fatty acids may be, but are not limited to palmitic, stearic, oleic, linoleic and linolenic acid.

The ODP1 polypeptide may comprise an amino acid sequence with at least 80%, 85%, 90%, 95% or 100% identity to SEQ ID NO: 69, SEQ ID NO: 81, or SEQ ID NO:111.

The Lec1 polypeptide may comprises an amino acid sequence with at least 80%, 85%, 90%, 95% or 100% identity to SEQ ID NO: 83, 94, 99, or 109.

The construct downregulating GAS activity may comprise all or part of nucleotide sequences encoding GAS1, GAS2 or GAS3 polypeptides or any combination thereof, wherein the nucleotide sequences encode amino acid sequences with at least 80%, 85%, 90%, 95% or 100% identity to SEQ ID NO: 139 (GAS3), SEQ ID NO: 140 (GAS1), or SEQ ID NO:143 (GAS2).

The second construct down regulating a fad 3 activity, a fad2 activity, or fat2B activity. The fad 2 activity may be encoded the nucleotide sequences encoding the amino acid sequences with at least 80%, 85%, 90%, 95% or 100% identity to SEQ ID NO: 119, 121, or 122.

The fad 3 activity may be encoded by the nucleotide sequences encoding the amino acid sequences with at least 80%, 85%, 90%, 95% or 100% identity to SEQ ID NO: 129, 131, or 133.

The fatB activity may be encoded by the nucleotide sequences encoding the amino acid sequences with at least 80%, 85%, 90%, 95% or 100% identity to SEQ ID NO: 135, or 137.

In some embodiments, the percent change of palmitic, linoleic and linolenic acid is a decrease when compared to control null segregant seeds. In yet another embodiment the percent change of oleic acid in the transgenic seed is an increase when compared to a control null segregant. The percent increase in oleic acid can be by at least 25% compared to a control null segregant seed. In some embodiments the percent increase in oleic acid is at least 300% when compared to a control null segregant seed. In an additional embodiment the percent change of total saturates is a decrease in the transgenic seed compared to control null segregant seeds. Additional embodiments include transgenic seed with percent decreases of palmitic, linoleic, linolenic acid, and total saturates and a percent increase of oleic acid when compared to control null segregant seeds.

Transgenic soybean seeds may also show a percent decrease in raffinose saccharides compared to control null segregant seeds in some embodiments. The percent decrease in raffinose saccharides can be by at least 60% compared to control null segregant seed.

Further embodiments include methods to achieve a percent increase in total fatty acids in the transgenic, protein content and alter (percent increase or percent decrease) the fatty acid composition of the transgenic seed comprising the polynucleotides and constructs described herein. The methods can also effect percent changes in the raffinose saccharide, the total saturate, the oleic acid, the palmitic acid, the linoleic acid, and the linolenic acid of the transgenic seeds compared to control seeds.

In one embodiment a method resulting in a percent increase of total fatty acids and a percent increase in protein in a soybean seed, the method comprising the steps of: crossing (i) a first transgenic soybean plant comprising at least one polynucleotide encoding a polypeptide selected from the group consisting of (i) a DGAT polypeptide, (ii) an ODP1 polypeptide, (iii) a Lec1 polypeptide, and (iv) a combination thereof, the polynucleotide being linked to at least one regulatory sequence; with (ii) a second transgenic soybean plant comprising a first construct down regulating a fad2 activity, and (b) selecting a third transgenic plant from the cross of step (a), wherein seed of the third transgenic plant comprises the first recombinant and the first construct and wherein expression of said first polypeptide and said first construct down regulating activity in said transgenic soybean seed results in a percent increase in protein in the transgenic soybean seed, when compared to the percent increase of control null segregant. The first construct down regulating activity may be one or more selected from the group consisting of a fad2, fad3, and fatB activity.

In one embodiment a method of producing a seed, the method comprising: (a) crossing (i) a first transgenic soybean plant comprising at least one polynucleotide encoding a polypeptide selected from the group consisting of (i) a DGAT polypeptide, (ii) an ODP1 polypeptide, (iii) a Lec1 polypeptide, and (iv) a combination thereof, the polynucleotide being linked to at least one regulatory sequence; with (ii) a second transgenic soybean plant comprising a first construct down regulating a fad2 activity, and (b) selecting a third transgenic plant from the cross of step (a), wherein seed of the third transgenic plant comprises the first recombinant and the first construct and wherein expression of said first polypeptide and said first construct down regulating activity in said transgenic soybean seed results in a percent increase in protein in the transgenic soybean seed, when compared to the percent increase of a control null segregant.

The polypeptide(s) and construct down-regulating activities may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

In yet another embodiment the at least one regulatory sequence is a soybean sucrose synthase promoter or Medicago truncatula sucrose synthase promoter.

The soybean sucrose synthase promoter may comprise a nucleic acid sequence selected from the group consisting of: (a) the nucleic acid sequence of SEQ ID NO: 91; (b) a nucleic acid sequence with at least 95% sequence identity to the nucleic acid sequence of SEQ ID NO: 91; (c) a nucleic acid sequence that hybridizes to SEQ ID NO: 91 under stringent conditions; and (d) a nucleic acid sequence comprising a functional fragment of (a), (b), or (c).

Furthermore the polynucleotides disclosed herein may be linked to a Medicago truncatula sucrose synthase promoter, wherein the Medicago truncatula sucrose synthase promoter comprises a nucleic acid sequence selected from the group consisting of: (a) the nucleic acid sequence of SEQ ID NO: 114 or SEQ ID NO: 117; (b) a nucleic acid sequence with at least 95% sequence identity to the nucleic acid sequence of SEQ ID NO: 114 or SEQ ID NO: 117; (c) a nucleic acid sequence that hybridizes to SEQ ID NO: 114 or SEQ ID NO: 117 under stringent conditions; and (d) a nucleic acid sequence comprising a functional fragment of (a), (b) or (c).

Transgenic soybeans produced by the methods described herein are also included.

Any of the transgenic seed described herein may comprise a recombinant construct having at least one DGAT sequence which can be selected from the group consisting of DGAT1, DGAT2 and DGAT1 in combination with DGAT2. Furthermore, the DGAT sequence can be a Yarrowia sequence or soybean sequence.

The DGAT1 polypeptide may comprise an amino acid sequence with at least 80%, 85%, 90%, 95% or 100% sequence identity to SEQ ID NO: 105. The second polynucleotide may encode a DGAT2 polypeptide. The DGAT2 polypeptide may comprise an amino acid sequence with at least 80%, 85%, 90%, 95% sequence identity to SEQ ID NO:107.

In another embodiment, a plant or a seed comprising any of the recombinant DNA constructs, polynucleotides or suppression constructs described herein is provided. The plant and the seed may be an oilseed plant and seed. The plant or seed may be a soybean plant or seed.

The percent increase in oil of the transgenic soybean seed may be at least 5%, 6%, 7%, 8%, 9%, 10%, 11%, 12%, 13%, 14%, 15%, 16%, 17%, 18%, 19%, 20%, 21%, 22%, 23%, 24%, 25%.

The percent increase in protein of the transgenic soybean seed may be at least 1%, 2%, 3%, 4%, 5%, 6%, or 7% compared to a control null segregant soybean seed.

The percent increase in protein of meal obtained from the transgenic soybean seed may be at least 3%, 4,%, 5%, 6%, 7%, 8%, 9%, 10%, 11% or 12% compared to meal obtained from control null segregant soybean seed.

Any of the transgenic seed may comprise a recombinant construct having downregulated GAS activity.

Also within the scope of the invention are product(s), such as for example meal) and/or by-product(s) (e.g. lecithin), and progeny, obtained from the transgenic soybean seeds of the invention. Oil and protein products obtained from the transgenic soybean of the invention are included as well as oil and protein products obtained by the methods of the invention.

The term “DAG AT” or “DGAT” refers to a diacylglycerol acyltransferase (also known as an acyl-CoA-diacylglycerol acyltransferase or a diacylglycerol O-acyltransferase) (EC 2.3.1.20). This enzyme is responsible for the conversion of acyl-CoA and 1,2-diacylglycerol to TAG and CoA (thereby involved in the terminal step of TAG biosynthesis). Two families of DAG AT enzymes exist: DGAT1 and DGAT2. The former family shares homology with the acyl-CoA:cholesterol acyltransferase (ACAT) gene family, while the latter family is unrelated (Lardizabal et al., J. Biol. Chem. 276(42):38862-28869 (2001)).

The term “PDAT” refers to a phospholipid:diacylglycerol acyltransferase enzyme (EC 2.3.1.158). This enzyme is responsible for the transfer of an acyl group from the sn-2 position of a phospholipid to the sn-3 position of 1,2-diacylglycerol, thus resulting in lysophospholipid and TAG (thereby involved in the terminal step of TAG biosynthesis). This enzyme differs from DGAT (EC 2.3.1.20) by synthesizing TAG via an acyl-CoA-independent mechanism.

The term “ARE2” refers to an acyl-CoA:sterol-acyltransferase enzyme (EC 2.3.1.26; also known as a sterol-ester synthase 2 enzyme), catalyzing the following reaction: acyl-CoA+cholesterol=CoA+cholesterol ester.

The term “Kennedy pathway enzyme genes” are defined as genes encoding enzymes that are involved in providing the immediate precursors for membrane lipid or storage lipid biosynthesis at the endoplasmic reticulum. Kennedy pathway enzymes also include enzymes that catalyze transfer of acyl groups between intermediates of membrane lipid or seed storage lipid biosynthesis at the endoplasmic reticulum (ER). Kennedy pathway enzyme can be soluble, cytosolic enzymes. They can be associated with the ER membrane system or they can be integral membrane proteins of the ER membrane system. A “Kennedy Pathway gene” is further defined as any gene directly involved biosynthesis or degradation of triacylglycerol (TAG) or TAG intermediates. Some examples of genes include glycerol-phosphate dehydrogenase (GPD), glycerol-phosphate acyltransferase (GPAT), glycerol acyltransferase, lyso-phospholipid acyltransferase (LPAT), lyso-phosphatidic acid acyltransferase (LPAAT), lyso-phosphatidylcholine acyltransferase (LPCAT), monoacylglyceride acyltransferase, phosphatidic acid phosphatase (PAP), lyso-phospholipid phospholipase, lyso-phosphatidic acid phospholipase, lyso-phosphatidylcholine phospholipase, phospholipase A1 (PLA1), phospholipase A2 (PLA2), phospholipase B (PLB), phospholipase C (PLC), phospholipase D (PLD), choline phosphotransferase (CPT), plastidic phosphoglucomutase (PGM), phospholipid:diacylglyceride acyltransferase (PDAT), lyso-phospholipid:diglyceride acyltransferase (LPDAT), triacylglyceride lipase, diacylglyceride lipase, monoacylglyceride lipase, and acylCoA binding protein (ACBP).

The oils can also be used as a blending source to make a blended oil product. By a blending source, it is meant that the oil described herein can be mixed with other vegetable oils to improve the characteristics, such as fatty acid composition, flavor, and oxidative stability, of the other oils. The amount of oil which can be used will depend upon the desired properties sought to be achieved in the resulting final blended oil product. Examples of blended oil products include, but are not limited to, margarines, shortenings, frying oils, salad oils, etc.

In another aspect, the oils described herein can be subjected to further processing such as hydrogenation, fractionation, interesterification or fat splitting (hydrolysis).

In still another aspect, by-products made during the production of the oils of are provided.

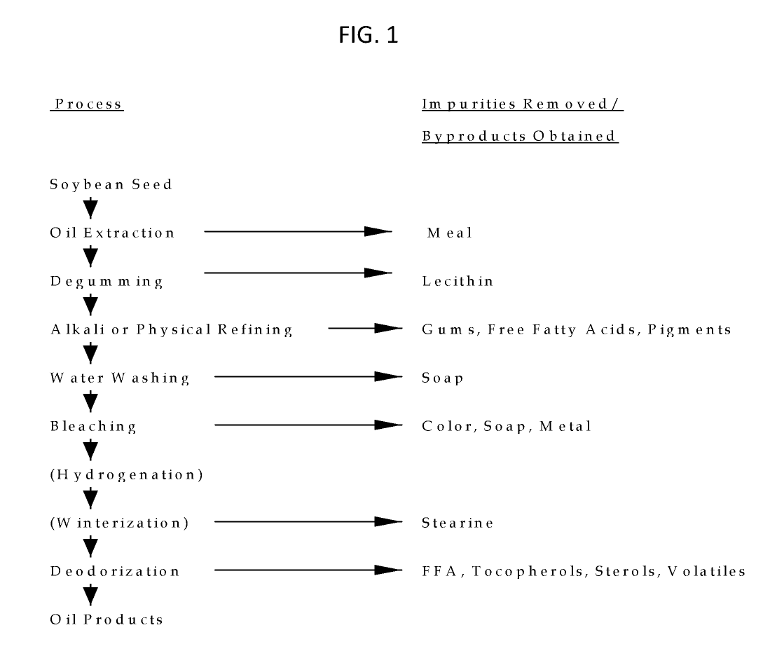

Methods for the extraction and processing of soybean seeds to produce soybean oil and meal are well known throughout the soybean processing industry. In general, soybean oil is produced using a series of steps which accomplish the extraction and purification of an edible oil product from the oil bearing seed. Soybean oils and soybean byproducts are produced using the generalized steps shown in FIG. 1.

Soybean seeds are cleaned, tempered, dehulled, and flaked which increases the efficiency of oil extraction. Oil extraction is usually accomplished by solvent (hexane) extraction but can also be achieved by a combination of physical pressure and/or solvent extraction. The resulting oil is called crude oil. The crude oil may be degummed by hydrating phospholipids and other polar and neutral lipid complexes which facilitate their separation from the nonhydrating, triglyceride fraction (soybean oil). The resulting lecithin gums may be further processed to make commercially important lecithin products used in a variety of food and industrial products as emulsification and release (antisticking) agents. Degummed oil may be further refined for the removal of impurities; primarily free fatty acids, pigments, and residual gums. Refining is accomplished by the addition of caustic which reacts with free fatty acid to form soap and hydrates phosphatides and proteins in the crude oil. Water is used to wash out traces of soap formed during refining. The soapstock byproduct may be used directly in animal feeds or acidulated to recover the free fatty acids. Color is removed through adsorption with a bleaching earth which removes most of the chlorophyll and carotenoid compounds. The refined oil can be hydrogenated resulting in fats with various melting properties and textures. Winterization (fractionation) may be used to remove stearine from the hydrogenated oil through crystallization under carefully controlled cooling conditions. Deodorization which is principally steam distillation so under vacuum is the last step and is designed to remove compounds which impart odor or flavor to the oil. Other valuable byproducts such as tocopherols and sterols may be removed during the deodorization process. Deodorized distillate containing these byproducts may be sold for production of natural vitamin E and other high value pharmaceutical products. Refined, bleached, (hydrogenated, fractionated) and deodorized oils and fats may be packaged and sold directly or further processed into more specialized products. A more detailed reference to soybean seed processing, soybean oil production and byproduct utilization can be found in Erickson, 1995, Practical Handbook of Soybean Processing and Utilization, The American Oil Chemists' Society and United Soybean Board.

Hydrogenation is a chemical reaction in which hydrogen is added to the unsaturated fatty acid double bonds with the aid of a catalyst such as nickel. High oleic soybean oil contains unsaturated oleic, linoleic, and linolenic fatty acids and each of these can be hydrogenated. Hydrogenation has two primary effects. First, the oxidative stability of the oil is increased as a result of the reduction of the unsaturated fatty acid content. Second, the physical properties of the oil are changed because the fatty acid modifications increase the melting point resulting in a semi-liquid or solid fat at room temperature.

There are many variables which affect the hydrogenation reaction which in turn alter the composition of the final product. Operating conditions including pressure, temperature, catalyst type and concentration, agitation and reactor design are parameters which can be controlled. Selective hydrogenation conditions can be used to hydrogenate the more unsaturated fatty acids in preference to the less unsaturated ones. Very light or brush hydrogenation is often employed to increase stability of liquid oils. Further hydrogenation converts a liquid oil to a physically solid fat. The degree of hydrogenation depends on the desired performance and melting characteristics designed for the particular end product. Liquid shortenings, used in the manufacture of baking products, solid fats and shortenings used for commercial frying and roasting operations, and base stocks for margarine manufacture are among the myriad of possible oil and fat products achieved through hydrogenation. A more detailed description of hydrogenation and hydrogenated products can be found in Patterson, H. B. W., 1994, Hydrogenation of Fats and Oils: Theory and Practice, The American Oil Chemists' Society.

Interesterification refers to the exchange of the fatty acyl moiety between an ester and an acid (acidolysis), an ester and an alcohol (alcoholysis) or an ester and ester (transesterification). Interesterification reactions are achieved using chemical or enzymatic processes. Random or directed transesterification processes rearrange the fatty acids on the triglyceride molecule without changing the fatty acid composition. The modified triglyceride structure may result in a fat with altered physical properties. Directed interesterfication reactions using lipases are becoming of increasing interest for high value specialty products like cocoa butter substitutes. Products being commercially produced using interesterification reactions include but are not limited to shortenings, margarines, cocoa butter substitutes and structured lipids containing medium chain fatty acids and polyunsaturated fatty acids. Interesterification is further discussed in Hui, Y. H., 1996, Bailey's Industrial Oil and Fat Products, Volume 4, John Wiley & Sons.

Fatty acids and fatty acid methyl esters are two examples of oleochemicals derived from vegetables oils. Fatty acids are used for the production of many products such as soaps, medium chain triglycerides, polyol esters, alkanolamides, etc. Vegetable oils can be hydrolyzed or split into their corresponding fatty acids and glycerine. Fatty acids produced from various fat splitting processes may be used crude or more often are purified into fractions or individual fatty acids by distillation and fractionation. Purified fatty acids and fractions thereof are converted into a wide variety of oleochemicals, such as dimer and trimer acids, diacids, alcohols, amines, amides, and esters. Fatty acid methyl esters are increasingly replacing fatty acids as starting materials for many oleochemicals such as fatty alcohols, alkanolamides, a-sulfonated methyl esters, diesel oil components, etc. Glycerine is also obtained by the cleavage of triglycerides using splitting or hydrolysis of vegetable oils. Further references on the commercial use of fatty acids and oleochemicals may be found in Erickson, D. R., 1995, Practical Handbook of Soybean Processing and Utilization, The American Oil Chemists' Society, and United Soybean Board; Pryde, E. H., 1979, Fatty Acids, The American Oil Chemists' Society; and Hui, Y. H., 1996, Bailey's Industrial Oil and Fat Products, Volume 4, John Wiley & Sons.

Soy protein products fall into three major groups. These groups are based on protein content, and range from 40% to over 90%. All three basic soy protein product groups (except full-fat flours) are derived from defatted flakes. They are the following: soy flours and grits, soy protein concentrates and soy protein isolates. These are discussed more fully below.

Additional embodiments include soy protein products with at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96% or 97% protein (N×6.25) on a moisture-free basis.

The soy protein products described herein can be incorporated into food, beverages, and animal feed.

The term “animal feed” refers to food that is given to animals, such as livestock and pets. Some feeds provide a healthy and nutritious diet, while others may be lacking in nutrients. Animals are given a wide range of different feeds, but the two major types of animal feed are processed animal feeds (compound feed) and fodder.

Compound feeds are feedstuffs that are blended from various raw materials and additives. The main ingredients used in commercially prepared feed are the feed grains, which include corn, soybeans, sorghum, oats, and barley. These blends are formulated according to the specific requirements of the target animal (including different types of livestock and pets).

They are manufactured by feed compounders as meal type, pellets or crumbles.

Compound feeds can be complete feeds that provide all the daily required nutrients, concentrates that provide a part of the ration (protein, energy) or supplements that only provide additional micro-nutrients such as minerals and vitamins.

Oxidation and therefore the shelf life of animal feed ingredients is a common problem in the industry. Oxidation is an irreversible chemical reaction in which oxygen reacts with feed and feed components and can result in decreased animal health and performance. The negative effects of oxidation can be seen in loss of palatability, degradation of the oil component, development of unwanted breakdown products, changes in color, and loss of energy. Meat obtained from animals grown on oxidized feed has significantly lower oxidative status compared to animals fed a feed that has not undergone significant oxidation. Meat from animals fed diets containing high oleic corn products show extended shelf life and greater oxidative stability (PCT Publication WO/2006/002052, published Jan. 5, 2006), particularly when combined with antioxidants such as tocols. Therefore it is highly desirable to prevent oxidation of feed and feed ingredients to protect both nutritional value and organoleptic quality.

Synthetic antioxidants are used to preserve feed quality by preventing the oxidation of lipids, which can lead to improved animal performance. Generally, synthetic antioxidants can act as free radical scavengers and thereby reduce lipid oxidation. Synthetic antioxidants can prolong animal feed shelf-life and protect nutritional and organoleptic quality

There are multiple methods to test the oxidation status of solid materials including soybean meal and other soybean protein products including accelerating aging methods which predict a material's shelf-life. One test which can be used is to age a material either at room temperature or elevated temperatures and to measure the oxidative status of the material at specific time points. The OSI instrument is useful in this regard in that it reflects the length of time needed to start the oxidation process known as the induction time. A longer induction time means that the material has greater oxidative stability and thereby shelf-life. Other methods include the measurement of volatiles and color change.

Methods for obtaining soy protein products are well known to those skilled in the art. For example soybean protein products can be obtained in a variety of ways. Conditions typically used to prepare soy protein isolates have been described by (Cho, et al, (1981) U.S. Pat. No. 4,278,597; Goodnight, et al. (1978) U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass ((1975) U.S. Pat. No. 897,574) and Campbell et al. ((1985) in New Protein Foods, ed. by Altschul and Wilcke, Academic Press, Vol., Chapter 10, Seed Storage Proteins, pp 302-338). The disclosures of each of these patents are herein incorporated by reference in their entireties.

“Soybean-containing products” or “Soy products” can be defined as those products containing/incorporating a soy protein product.

For example, “soy protein products” can include, and are not limited to, those items listed in Table 1.

TABLE 1
Soy protein products derived from soybean seeds^{a}

| Whole Soybean Products | Processed Soy Protein Products |
|---|---|
| Roasted Soybeans | Full Fat and Defatted Flours |
| Baked Soybeans | Soy Grits |
| Soy Sprouts | Soy Hypocotyls |
| Soy Milk | Soybean Meal |
| | Soy Milk |
| | Soy Milk Powder |
| | Soy Protein Isolates |
| Specialty Soy Foods/Ingredients | |
| Soy Milk | Soy Protein Concentrates |
| Tofu | Textured Soy Proteins |
| Tempeh | Textured Flours and Concentrates |
| Miso | Textured Concentrates |
| Soy Sauce | Textured Isolates |
| Hydrolyzed Vegetable Protein | Soy Crisps |
| Whipping Protein | |

^{a}See Soy Protein Products: Characteristics, Nutritional Aspects and Utilization (1987). Soy Protein Council.

“Processing” refers to any physical and chemical methods used to obtain the products listed in Table 1 and includes, and is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992).

Defatted flakes refer to flaked, dehulled cotyledons that have been defatted and treated with controlled heat to remove the remaining hexane. This term can also refer to a flour or grit that has been ground.

“White” flakes refer to flaked, dehulled cotyledons that have been defatted and treated with controlled heat to remove the remaining hexane. This term can also refer to a flour that has been ground.

“Grits” refer to defatted, dehulled cotyledons having a U.S. Standard screen size of between No. 10 and 80.

“Soy Protein Concentrates” refer to those products produced from dehulled, defatted soybeans and typically contain 65 wt % to 90 wt % soy protein on a moisture free basis. Soy protein concentrates are typically manufactured by three basic processes: acid leaching (at about pH 4.5), extraction with alcohol (about 55-80%), and denaturing the protein with moist heat prior to extraction with water. Conditions typically used to prepare soy protein concentrates have been described by Pass (1975) U.S. Pat. No. 3,897,574 (herein incorporated by reference in its entirety); Campbell et al., (1985) in New Protein Foods, ed. by Altschul and Wilcke, Academic Press, Vol. 5, Chapter 10, Seed Storage Proteins, pp 302-338).

As used herein, the term “soy protein isolate” or “isolated soy protein” refers to a soy protein containing material that contains at least 90% soy protein by weight on a moisture free basis.

“Extrusion” refers to processes whereby material (grits, flour or concentrate) is passed through a jacketed auger using high pressures and temperatures as a means of altering the texture of the material. “Texturing” and “structuring” refer to so extrusion processes used to modify the physical characteristics of the material. The characteristics of these processes, including thermoplastic extrusion, have been described previously (Atkinson (1970) U.S. Pat. No. 3,488,770 (herein incorporated by reference in its entirety), Horan (1985) In New Protein Foods, ed. by Altschul and Wilcke, Academic Press, Vol. 1A, Chapter 8, pp 367-414). Moreover, conditions used during extrusion processing of complex foodstuff mixtures that include soy protein products have been described previously (Rokey (1983) Feed Manufacturing Technology III, 222-237; McCulloch, U.S. Pat. No. 4,454,804; herein incorporated by reference in its entirety).

The oils disclosed herein can be used in a variety of applications, including in the preparation of foods. Examples include, but are not limited to, uses as ingredients, as coatings, as salad oils, as spraying oils, as roasting oils, and as frying oils. Foods in which the oil may be used include, but are not limited to, crackers and snack foods, confectionery products, syrups and toppings, sauces and gravies, soups, batter and breading mixes, baking mixes and doughs. Foods which incorporate the oil may retain better flavor over longer periods of time due to the improved stability against oxidation imparted by this oil.

In another aspect, soybean oil described herein can be used in industrial applications. Soybean oils described herein can be low in polyunsaturates and have high oxidative stability and high temperature stability. These oils are desirable for industrial applications such as an industrial fluid, for example as an industrial lubricant or as a hydraulic fluid, etc. Additives which can be used to make industrial lubricants and hydraulic fluids are commercially available, including additives specially formulated for use with high oleic vegetable oils. Additives generally contain antioxidants and materials which retard foaming, wear, rust, etc.

One common method for measuring oxidative stability of industrial fluids is the rotary bomb oxidation test (ASTM D-2272). The performance of the oil of this invention when compared to commercially available products using the rotary bomb oxidation test is set forth in the example below.

Residual fatty acid analysis. The commercial process used to de-fat soy flakes with hexane leaves a residue of fatty acids that can act as substrate for generation of off-flavor compounds. Depending on the method of analysis, the residual fat content of hexane-defatted soy flakes can range from, 0.6-1.0% (W:W) (ether extractable; AOCS Method 920.39 (Official Methods of Analysis of the AOAC International (1995), 16^{th} Edition, Method 920.39C, Locator #4.2.01 (modified)) to 2.5-3% (W:W) (acid hydrolysable; AOAC Method 922.06 (Official Methods of Analysis of the AOAC International (1995), 16^{th} Edition, Method 922.06, Locator 32.1.13 (modified)). The principle reason for the discrepancy between these two methods of estimating residual fatty acids is the chemical nature of the fat classes associated with the protein matrix after hexane extraction. A small proportion of the residual fatty acid is in the form of neutral lipid (i.e., triglyceride) and the remainder is present as polar lipid (e.g., phospholipids, a.k.a., lecithin). Because of its polar nature the phospholipid is inaccessible to ether extraction and is only removed from the protein matrix if acid hydrolysis or some other stringent extraction protocol is performed. Therefore, the ether extraction technique gives an estimation of the neutral lipid fraction whereas the acid hydrolysable method gives a better estimate of the total residual fatty acid content (i.e., neutral and polar fractions).

Both of the AOAC methods described above rely on gravimetric determinations of the residual fatty acids and, although in combination they give an indication of the fat classes (neutral vs. polar), such estimates are crude and are subject to interference from other hydrophobic materials (e.g. saponins). Further, no information is obtained on the fatty acid composition and how it may have been affected by various experimental treatments or by the genetics of the starting material. AOAC methods for the determination of the fatty acid composition of residual fatty acids are available (Official Methods of Analysis of the AOAC International (2000), 17^{th} Edition, Method 983.23 Locator 45.4.02, Method 969.33 Locator 41.1.28, Method 996.06 Locator 41.1.28A). These are based on the conversion of residual fatty acids, extracted by acid hydrolysis, to fatty acid methyl esters prior to analysis by gas chromatography. Such techniques are rarely used to assess the residual fatty acid content of food materials in commercial settings although they are used for fatty acid evaluations in support of nutritional labeling. A report in which these methods have been used to determine the residual fatty acid composition of commercial soy protein isolates has recently been published (Solina et al. (2005) Volatile aroma components of soy protein isolate and acid-hydrolysed vegetable protein Food Chemistry 90: 861-873)

Also disclosed are food, food supplements, food bars, and beverages as well as animal feed (such as pet foods) that have incorporated therein a soybean protein product described herein. The beverage can be in a liquid or in a dry powdered form.

The foods to which the soybean protein product described herein can be incorporated or added include almost all foods, beverages and feed (such as pet foods). For example, there can be mentioned food supplements, food bars, meats such as meat alternatives, ground meats, emulsified meats, marinated meats, and meats injected with a soybean protein product. Included may be beverages such as nutritional beverages, sports beverages, protein-fortified beverages, juices, milk, milk alternatives, and weight loss beverages. Mentioned may also be cheeses such as hard and soft cheeses, cream cheese, and cottage cheese. Included may also be frozen desserts such as ice cream, ice milk, low fat frozen desserts, and non-dairy frozen desserts. Finally, yogurts, soups, puddings, bakery products, salad dressings, spreads, and dips (such as mayonnaise and chip dips) may be included.

A soy protein product can be added in an amount selected to deliver a desired amount to a food and/or beverage. The terms “soybean protein product” and “soy protein product” are used interchangeably herein.

Any of the transgenic soybean seeds described herein can be used as a source of a protein product.

The oils and protein products (such as for example meal) described herein can also be used as a blending source to make a blended oil or protein product. By a blending source, it is meant that the oil can be mixed with other vegetable oils to improve the characteristics, such as fatty acid composition, flavor, and oxidative stability, of the other oils. Examples of blended oil products include, but are not limited to, margarines, shortenings, frying oils, salad oils, etc.

The blending source for a protein product can be another protein product to improve the characteristics of the blended product, such as lower raffinose saccharides, increase sucrose, protein etc. or increased stability due to presence of residual fatty acids, such as increased amounts of oleic acid.

Soybeans with decreased levels of saturated fatty acids have been described resulting from mutation breeding (Erickson et al. (1994) J. Hered. 79:465-468; Schnebly et al. (1994) Crop Sci. 34:829-833; and Fehr et al. (1991) Crop Sci. 31:88-89) and transgenic modification (U.S. Pat. No. 5,530,186 herein incorporated by reference in its entirety).

Two soybean fatty acid desaturases, designated FAD2-1 and FAD2-2, are Δ-12 desaturases that introduce a second double bond into oleic acid to form linoleic acid, a polyunsaturated fatty acid. FAD2-1 is expressed only in the developing seed (Heppard et al. (1996) Plant Physiol. 110:311-319). The expression of this gene increases during the period of oil deposition, starting around 19 days after flowering, and its gene product is responsible for the synthesis of the polyunsaturated fatty acids found in soybean oil. GmFad 2-1 is described in detail by Okuley, J. et al. (1994) Plant Cell 6:147-158 and in WO94/11516. It is available from the ATCC in the form of plasmid pSF2-169K (ATCC accession number 69092). FAD 2-2 is expressed in the seed, leaf, root and stem of the soy plant at a constant level and is the “housekeeping” 12-desaturase gene. The Fad 2-2 gene product is responsible for the synthesis of polyunsaturated fatty acids for cell membranes.

Since FAD2-1 is the major enzyme of this type in soybean seeds, reduction in the expression of FAD2-1 results in increased accumulation of oleic acid (18:1) and a corresponding decrease in polyunsaturated fatty acid content.

Reduction of expression of FAD2-2 in combination with FAD2-1 leads to a greater accumulation of oleic acid and corresponding decrease in polyunsaturated fatty acid content.

FAD3 is a Δ-15 desaturase that introduces a third double bond into linoleic acid (18:2) to form linolenic acid (18:3). Reduction of expression of FAD3 in combination with reduction of FAD2-1 and FAD2-2 leads to a greater accumulation of oleic acid and corresponding decrease in polyunsaturated fatty acid content, especially linolenic acid.

Nucleic acid fragments encoding FAD2-1, FAD2-2, and FAD3 have been described in WO 94/11516 and WO 93/11245. Chimeric recombinant constructs comprising all or a part of these nucleic acid fragments or the reverse complements thereof operably linked to at least one suitable regulatory sequence can be constructed wherein expression of the chimeric gene results in an altered fatty acid phenotype. A chimeric recombinant construct can be introduced into soybean plants via transformation techniques well known to those skilled in the art.

Transgenic soybean plants resulting from a transformation with a recombinant DNA are assayed to select plants with altered fatty acid profiles. The recombinant construct may contain all or part of 1) the FAD2-1 gene or 2) the FAD2-2 gene or 3) the FAD3 gene or 4) combinations of all or portions of the FAD2-1, Fad2-2, or FAD3 genes.

Recombinant constructs comprising all or part of 1) the FAD2-1 gene with or without 2) all or part of the Fad2-2 gene with or without all or part of the FAD3 gene can be used in making a transgenic soybean plant having a high oleic phenotype. An altered fatty acid profile, specifically an increase in the proportion of oleic acid and a decrease in the proportion of the polyunsaturated fatty acids, indicates that one or more of the soybean seed FAD genes (FAD2-1, Fad2-2, FAD3) have been suppressed. Assays may be conducted on soybean somatic embryo cultures and seeds to determine suppression of FAD2-1, Fad2-2, or FAD3.

A transgenic soybean seed is provided having an increased total fatty acid content of at least 10%, an increased protein content of at least 1% and an altered (increased or decreased) fatty acid content of at least one fatty acid when compared to a control null segregant. The recombinant DNA construct(s) comprise at least one poly-nucleotide encoding a polypeptide selected from the group consisting of: a DGAT polypeptide, an ODP1 polypeptide, and a Lec1 polypeptide, alone or in combination with a construct downregulating GAS activity, alone or in combination with at least one construct downregulating activity selected from the group consisting of: a fad 3 activity, a fad2 activity and fat2B activity. The recombinant constructs can be in the same or in separate recombinant construct(s), linked to at least one regulatory sequence. Fatty acids may be oleic, stearic, palmitic, linoleic and linolenic acid.

In some embodiments, the level of palmitic, linoleic and linolenic acid is decreased when compared to control null segregant. In yet another embodiment, the level of oleic acid in the transgenic seed is increased when compared to a control null segregant. The increase in oleic acid can be increased by at least 25% compared to a control null segregant seed. In yet another embodiment the oleic acid content can be increased by at least 300% compared to a control seed. In an additional embodiment the level of total saturates is decreased In the transgenic seed. Additional embodiments include transgenic seed with decreased levels of palmitic, linoleic and linolenic acid, decreased total saturates and increased oleic acid levels when compared control null segregant. The level of raffinose saccharides may also decreased compared to control null segregant seeds in some embodiments. The decrease in raffinose saccharides can be by at least 60% compared to control null segregant seed. Further embodiments include methods to increase the total fatty acid content, protein content and alter (increase or decrease) the fatty acid composition of the transgenic seed comprising the polynucleotides and constructs described herein. The methods can also include alterations in the raffinose saccharide content, the total saturate content, the oleic acid content, the palmitic acid content, the linoleic acid content, and linolenic acid content of the transgenic seeds. In yet another embodiment the at least one regulatory sequence is a soybean sucrose synthase promoter or Medicago truncatula sucrose synthase promoter. Transgenic soybeans produced by the methods described herein are also included.

Any of the transgenic seed disclosed herein may comprise a recombinant construct having at least one DGAT sequence which can be selected from the group consisting of DGAT1, DGAT2 and DGAT1 in combination with DGAT2. Furthermore, the DGAT sequence can be a Yarrowia sequence or soybean sequence.

Any of the transgenic seed disclosed herein may comprise a recombinant construct having downregulated GAS activity.

Also within the scope of the invention are product(s), such as meal and/or by-product(s), such as lecithin, and progeny, obtained from the transgenic soybean seeds disclosed herein.

Transgenic soybean seed is provided exhibiting an at least 10% increase in total fatty acids when compared to a control null segregant soybean seed. It is understood that any measurable percent increase in the total fatty acids of a transgenic versus a non-transgenic, null segregant would be useful. Such percent increases in the total fatty acids may include, but are not limited to, at least 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

Transgenic soybean seed is provided exhibiting a percent increase in protein of at least 1% when compared to a control null segregant seed. It is understood that any measurable percent increase protein in a transgenic versus control null segregant would be useful. Such percent increase in the protein may include, but are not limited to, at least 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%. 5.4%, 5.5.%, 5.6%, 5.7%, 5.8%, 5.9%, 6.0%, 6.1%, 6.2%, 6.3%, 6.4%, 6.5%, 7.0%, 7.5%, 8.0%, 8.5%, 8.6%, 8.7%, 8.8.%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 125%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3% 14.4%, 14.5%, 14.6%, 14.7%, 14.8%, 14.9%, or 15.0%.

In another embodiment, meals are obtained from the transgenic soybean seed exhibiting an at least 1% increase in protein when compared to control meal obtained from a control null segregant soybean seed. It is understood that any measurable percent increase of protein in meals(s) obtained from a transgenic versus a control null segregant seed would be useful. Such percent increase in the protein may include, but are not limited to, at least 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%. 5.4%, 5.5.%, 5.6%, 5.7%, 5.8%, 5.9%, 6.0%, 6.1%, 6.2%, 6.3%, 6.4%, 6.5%, 7.0%, 7.5%, 8.0%, 8.5%, 8.6%, 8.7%, 8.8.%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 125%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3% 14.4%, 14.5%, 14.6%, 14.7%, 14.8%, 14.9%, or 15.0%.

Meals obtained by the methods described herein exhibiting a percent increase of protein compared to a control meal obtained from a null segregant may include, but are not limited to percent increase of protein of at least 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%. 5.4%, 5.5.%, 5.6%, 5.7%, 5.8%, 5.9%, 6.0%, 6.1%, 6.2%, 6.3%, 6.4%, 6.5%, 7.0%, 7.5%, 8.0%, 8.5%, 8.6%, 8.7%, 8.8.%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 125%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3% 14.4%, 14.5%, 14.6%, 14.7%, 14.8%, 14.9%, or 15.0%.

Transgenic soybean seed having altered (increased or decreased) fatty acid content when compared to the fatty acid content of control null segregant soybean seed are provided. Fatty acids altered may be oleic, stearic, palmitic, linoleic and linolenic acid.

It is understood that any measurable alteration (increase or decrease) in the total fatty acid content of a transgenic versus a control null segregant seed would be useful.

A percent decrease of palmitic acid in a transgenic versus a control null segregant may include, but is not limited to, at least 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96%, 97%, 98%, 99%, or 100%.

A percent decrease of stearic acid in a transgenic versus a control null segregant may include, but is not limited to, at least 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, or 50.0%.

A percent increase of stearic acid in a transgenic versus a control null segregant may include, but is not limited to, at least 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, or 50.0%.

A percent increase of oleic acid in a transgenic versus a control null segregant may include, but is not limited to, at least 20%, 25%, 30%, 35%, 40%, 45%, 50%, 55%, 60%, 65%, 70%, 75%, 80%, 85%, 90%, 95%, 100%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, or 500%.

A percent decrease in linoleic acid content of a transgenic versus control a control null segregant may include, but is not limited to, at least 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96%, 97%, 98%, 99%, or 100%.

A percent increase of linoleic acid in a transgenic versus a control null segregant may include, but is not limited to, at least 0.5%, 1.0%, 3.0%, 4.0%, 5.0%, 6.0%, 7.0%, 8.0%, 9.0%, or 10.0%.

A percent decrease of linolenic acid in a transgenic versus a control null segregant may include, but is not limited to, at least 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96%, 97%, 98%, or 99%.

A percent decrease in total saturates (saturated fatty acids) in a transgenic versus a control null segregant may include, but is not limited to, at least 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, or 80.0%.

A percent decrease of total raffinosaccharides in a transgenic versus a control such as a non-transgenic, null segregant seed may include, but is not limited to, at least 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96%, 97%, 98%, or 99%.

A percent decrease of total carbohydrates in a transgenic versus a control null segregant seed may include, but is not limited to, at least 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, or 40.0%.

A percent increase of total sucrose in a transgenic versus a control null segregant may include, but is not limited to, at least 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, or 40.0%.

In some cases no percent change of protein in the transgenic compared to a control null segregant seed or a percent decrease of protein may be observed. The percent decrease of protein in the transgenic seed compared to the a control null segregant seed may be at least 1%, 2%, 3%, 4%, 5%, 6%, 7%, 8%, 9% or 10%.

In some embodiments the sum of the percent increase of total fatty acids (oil) and the percent increase of protein in the transgenic versus a control null segregant seed may include, but is not limited to, at least 1.0%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 21.0 22.0%, 23.0%, 24.0%, 25.0%, 26.0%, 27.0%, 28.0%, 29.0%, 30.0%, 31.0%, 32.0%, 33.0%, 34.0%, 35.0%, 36.0%, 37.0%, 38.0%, 39.0%, or 40.0%.

In some embodiments, transgenic seed(s) exhibit a percent decrease of palmitic, linoleic and linolenic acid when compared to control seed(s). In yet another embodiment transgenic seed(s) exhibit a percent increase of oleic acid when compared to a control null segregant seed. The percent increase of oleic acid can be by at least 25% compared to a control null segregant seed. In an additional embodiment transgenic seed (s) exhibit a percent decrease of total compared to a control null segregant seed(s), It is understood that any measurable percent change of total fatty acids (oil) in a transgenic versus a control null segregant seed would be useful. Furthermore, any percent increases of protein in a transgenic versus a control null segregant seed(s) would be useful, such percent increases in the protein may include, but are not limited to, at least 0.8%, 0.9% 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%. 5.4%, 5.5.%, 5.6%, 5.7%, 5.8%, 5.9%, 6.0%, 6.1%, 6.2%, 6.3%, 6.4%, 6.5%, 7.0%, 7.5%, 8.0%, 8.5%, 8.6%, 8.7%, 8.8.%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 125%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3% 14.4%, 14.5%, 14.6%, 14.7%, 14.8%, 14.9%, or 15.0%.

It will be apparent to those of skill in the art that variations may be applied to the compositions and methods described herein and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

It is to be understood that the invention is not limited in its application to the details of construction and the arrangement of components set forth in the following description. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

All patents and patent applications mentioned in this application are incorporated by reference herein in their entireties for all purposes. In case of conflict between the present disclosure and that of a patent or publication incorporated by reference, the present disclosure controls.

The following non-limiting examples are purely illustrative.

### EXAMPLES

In the following Examples parts and percentages are by weight and degrees are Celsius, unless otherwise stated.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micro molar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kB” means kilobase(s).

### Example 1

### Constructs for Generating Soybean Lines with Seed-Targeted Silencing of Galactinol Synthase and Fad3 and Seed Targeted Over-Expression of DGAT Enzymes

Using standard PCR and cloning methods, a 776 by fragment of the soy annexin promoter (U.S. Pat. No. 7,129,089, issued Oct. 31, 2006) was combined with the NotI fragment of pKR1756 (SEQ ID NO:1), containing the 159-fad3c amiRNA precursor (SEQ ID NO:2) and the soy BD30 transcription terminator from pKR268 (U.S. Pat. No. 8,013,215, issued Sep. 6, 2011) resulting in the BsiWI/SbfI fragment of SEQ ID NO:3 (called Ann-fad3c-BD30).

The Ann-fad3c-BD30 SbfI/BsiWI fragment (SEQ ID NO:3) was cloned into the SbfI/BsiWI fragment of pKR277 (SEQ ID NO:4) to produce pKR1850 (SEQ ID NO:5).

Stacking fad3c amiRNA Cassette with YLDGAT2

Construction of a plasmid containing the Yarrowia DGAT2 gene flanked by the soy beta-conglycinin promoter and the phaseolin terminator was described for KS362 (SEQ ID NO:6) in Example 4 of U.S. Pat. No. 8,143,473; issued Mar. 27, 2012. Using standard PCR, restriction digest and cloning methods, BsiWI restriction sites were added flanking the beta-conglycin/YLDGAT2/phaseolin cassette of KS362 (SEQ ID NO:6), resulting in the BsiWI fragment of SEQ ID NO:7

The BsiWI fragment containing the beta-conglycin/YLDGAT2/phaseolin cassette (SEQ ID NO:7) was cloned into the BsiWI site of pKR1850 (SEQ ID NO:5) to produce pKR1975 (SEQ ID NO:8).

Site-Specific Integration Donor Vector Stacking the fad3c amiRNA and Galactinol Synthase Silencing Cassettes with YLDGAT2

The yeast FLP/FRT site specific recombination system has been shown to function in plants. Earlier, the system was utilized for excision of unwanted DNA. See, Lyznik et al. (1993) Nucleic Acid Res. 21:969-975. Subsequently, non-identical FRTs were used for the exchange, targeting, arrangement, insertion and control of expression of nucleotide sequences into the plant genome (PCT Publication No. WO1999025821; PCT Publication No. WO1999025840; PCT Publication No. WO1999025854; PCT Publication No. 1999025855; and PCT Publication No. WO2007011733; the contents of all of which are herein incorporated by reference).

Constructs and methods for FLP/FRT site specific recombination to achieve recombinase mediated cassette exchange (RMCE) for stacking gene cassettes in soy are described in U.S. Pat. No. 8,293,533 issued Oct. 23, 2012, the contents of which are herein incorporated by reference.

Using standard PCR and cloning methods by one skilled in the art, the following DNA elements were assembled to produce a 5195 by basic donor construct QC632 (SEQ ID NO:9).

Sequence 70-117 of QC632 (SEQ ID NO:9) is a FLP recombinase recognition site FRT1 (U.S. Pat. No. 8,293,533 issued Oct. 23, 2012). Sequence 132-2087 is the soybean acetolactate synthase (als) gene coding region encoding a mutant ALS enzyme insensitive to sulfonylurea herbicides and having a P178A mutation in the encoded protein (described in U.S. Pat. No. 5,378,824, issued Jan. 3, 1995). Sequence 2104-2414 is the potato proteinase II inhibitor gene (PINII) terminator (SEQ 10). Sequence 2471-2518 is a FLP recombinase recognition site FRT6 (described in U.S. Pat. No. 8,293,533 issued Oct. 23, 2012). Sequence 2608-2655 is a FLP recombinase recognition site FRT87 (described previously in U.S. Pat. No. 8,293,533 issued Oct. 23, 2012). Sequence 2668-5189 is vector backbone (described previously in U.S. Pat. No. 8,293,533 issued Oct. 23, 2012) containing the T7 promoter (sequence 3903-3998), the hygromycin phosphotransferase (hpt) gene coding region (sequence 3999-5021) and the T7 terminator (sequence 5046-5178).

Plasmid QC632 (SEQ ID NO:9) was digested with SmaI and EcoRV in order to remove the FLP recombinase recognition FRT6 site. The remaining 5193 bp fragment was re-ligated to produce pKR1763 (SEQ ID NO:11).

Using standard PCR, restriction digests and cloning techniques, a DNA fragment of the 3′ transcription terminator region of the phaseolin gene with flanking ORFstop sequences (ORFstopA and ORFstopB as well as flanking BsiWi/MluI sites (SEQ ID NO:12) was produced, digested with BsiWI/MluI and cloned into the AscI/Acc651 of pKR1763 (SEQ ID NO:11) to produce pKR1849 (SEQ ID NO:13).

A unique SbfI site in pKR1849 (SEQ ID NO:13) was removed by digestion with SbfI, 51 nuclease treatment and re-ligation to produce pKR1857 (SEQ ID NO:14).

Donor construct pKR1857 (SEQ ID NO:14) is a 6341 by construct comprising the following DNA elements.

Sequence 45-92 is a FLP recombinase recognition site FRT1. Sequence 107-2062 is the soybean acetolactate synthase (als) gene coding region encoding a mutant ALS enzyme insensitive to sulfonylurea herbicides and having a P178A mutation in the encoded protein. Sequence 2079-2389 is the potato proteinase II inhibitor gene (PINII) terminator. Sequence 2425-2440 is a sequence of DNA comprising ORF stop codons in all 6 frames (ORFSTOP-A). Sequence 2443-3612 is the phaseolin transcription terminator. Sequence 3644-3660 is a sequence of DNA comprising ORF stop codons in all 6 frames (ORFSTOP-B). Sequence 3733-3780 is a FLP recombinase recognition site FRT87. Sequence 3793-6314 is vector backbone containing the T7 promoter (sequence 5028-5123), the hygromycin phosphotransferase (hpt) gene coding region (sequence 5124-6146) and the T7 terminator (sequence 6171-6303).

The AscI fragment of pKR1975 (SEQ ID NO:8), containing the fad3c amiRNA and YLDGAT2 cassettes, was cloned into the AscI site of pKR1857 (SEQ ID NO:14) to produce pKR1980 (SEQ ID NO:15).

A hairpin construct comprising polynucleotide fragments of the galactinol synthase 1 (GAS1, described in Applicants' Assignee's U.S. Pat. No. 5,648,210; Issued Jul. 15, 1997), galactinol synthase 2 (GAS2; Applicants' Assignee's U.S. Pat. No. 6,967,262; Issued Nov. 22, 2005) and galactinol synthase 3 (GAS3; described in Applicants' Assignee's U.S. Pat. No. 7,294,756 B2; Issued Nov. 11, 2007) in the stem structure and a potato ST-LS1 intron2 in the loop structure was produced by standard PCR methods, similar to those described in Example 27, resulting in the Not1 fragment of SEQ ID NO:16 (called Gas123 hp).

The NotI fragment containing Gas123 hp (SEQ NO:16) was cloned into the NotI site of pKR1273 (SEQ ID NO:17) to produce pKR1292 (SEQ ID NO:18). In pKR1292 (SEQ ID NO:18), the Gas123 hp (SEQ NO:16) is cloned behind the soy KTi promoter and the complete cassette is flanked by SbfI restriction enzyme sites.

The SbfI fragment of pKR1292 (SEQ ID NO:18) was cloned into the SbfI site of pKR1980 (SEQ ID NO: 15) to produce pKR1986 (SEQ ID NO:19). In this way, YLDGAT2 overexpression and fad3 and galactinol synthase gene silencing cassettes were stacked together in one SSI donor construct. Plasmid pKR1986 (SEQ ID NO:19) was also given the designation PHP50573.

### Constructing a FLP Recombinase Expression Plasmid (PHP44664)

The construction of the 4860 by FLP recombinase expression plasmid QC292 (SEQ ID NO:20) was described previously in U.S. Pat. No. 8,293,533 issued Oct. 23, 2012.

Using common methods familiar to one skilled in the art, the ampicillin selection fragment of QC292 (SEQ ID NO:20) was replaced with a hygromycin selection fragment to produce QC608 (SEQ ID NO:21). Plasmid QC608 (SEQ ID NO:21) was also given the designation PHP44664.

In PHP44664 (SEQ ID NO:21), sequence 47-532 is the constitutive promoter SCP1. Sequence 539-611 is the OMEGA 5′ UTR. Sequence 626-1897 is a codon optimized FLP recombinase coding region. Sequence 1904-2213 is the PINII terminator. Sequence 2214-4747 is vector backbone containing the T7 promoter (sequence 3455-3550), the hygromycin phosphotransferase (hpt) gene coding region (sequence 3551-4573) and the T7 terminator (sequence 4598-4730).

### Example 2

### Raffinose Family Oligosaccharide (RFO) Analysis in Transgenic Soybean Somatic Embryos and Soybean Seeds

Individual immature soybean embryos were dried-down (by transferring them into an empty small Petri dish that was seated on top of a 10 cm Petri dish containing some agar gel to allow slow dry down) to mimic the last stages of soybean seed development. Dried-down embryos are capable of producing plants when transferred to soil or soil-less media. Storage products produced by embryos at this stage are similar in composition to storage products produced by zygotic embryos at a similar stage of development. The storage product profile is predictive of plants derived from a somatic embryo line (PCT Publication No. WO 94/11516, published on May 26, 1994). Raffinose Family Oligosaccharides (raffinose, stachyose) of transgenic somatic embryos containing recombinant expression construct of the invention were measured by thin layer chromatography. Somatic embryos were extracted with hexane then dried. The dried material was re-suspended in 80% methanol, incubated at room temperature for 1-2 hours, centrifuged, and 2 μl of the supernatant is spotted onto a TLC plate (Kieselgel 60 CF, from EM Scientific, Gibbstown, N.J.; Catalog No. 13749-6). The TLC was run in ethylacetate: isopropanol:20% acetic acid (3:4:4) for 1-1.5 hours. The air dried plates were sprayed with 2% sulfuric acid and heated until the charred sugars were detected. Somatic embryos expressing the GAS suppression construct showed reduced levels of raffinose sugars (raffinose and stachyose) when compared to untransformed wild type soybean (WT) somatic embryos.

Mature soybean T1 and T2 seeds derived from events expressing the GAS suppression construct were chipped and the chips were analyzed by TLC as described above. Seed from derived from events expressing the GAS construct showed reduced levels of raffinose sugars (raffinose and stachyose) when compared to untransformed wild type soybean (WT) seeds.

### Example 3

### Analysis of Seed Oil Content

### NMR Based Analysis of Seed Oil Content and Fatty Acid Composition Determined by GC-FAME:

Seed oil content was determined using a Maran Ultra NMR analyzer (Resonance Instruments Ltd, Whitney, Oxfordshire, UK). Samples (either individual soybean seed or batches of Arabidopsis seed ranging in weight between 5 and 200 mg) were placed into pre-weighed 2 mL polypropylene tubes (Corning Inc, Corning N.Y., USA; Part no. 430917) previously labeled with unique bar code identifiers. Samples were then placed into 96 place carriers and processed through the following series of steps by an Adept Cobra 600 SCARA robotic system.
1. pick up tube (the robotic arm was fitted with a vacuum pickup devise)
2. read bar code
3. expose tube to antistatic device (ensured that Arabidopsis seed were not adhering to the tube walls)
4. weigh tube (containing the sample), to 0.0001 g precision.
5. NMR reading; measured as the intensity of the proton spin echo 1 msec after a 22.95 MHz signal had been applied to the sample (data was collected for 32 NMR scans per sample)
6. return tube to rack
7. repeat process with next tube
   Bar codes, tubes weights and NMR readings were recorded by a computer connected to the system. Sample weight was determined by subtracting the polypropylene tube weight from the weight of the tube containing the sample.

Seed oil content of soybeans seed was calculated as follows:

$$\% oil ( \% wt basis ) = \frac{( NMR signal / sample wt ( g ) ) - 70.58 )}{351.45}$$

Calibration parameters were determined by precisely weighing samples of soy oil (ranging from 0.0050 to 0.0700 g at approximately 0.0050 g intervals; weighed to a precision of 0.0001 g) into Corning tubes (see above) and subjecting them to NMR analysis. A calibration curve of oil content (% seed wt basis; assuming a standard seed weight of 0.1500 g) to NMR value was established.

The relationship between seed oil contents measured by NMR and absolute oil contents measured by classical analytical chemistry methods was determined as follows. Fifty soybean seed, chosen to have a range of oil contents, were dried at 40° C. in a forced air oven for 48 h. Individual seeds were subjected to NMR analysis, as described above, and were then ground to a fine powder in a GenoGrinder (SPEX Centriprep (Metuchen, N.J., U.S.A.); 1500 oscillations per minute, for 1 minute). Aliquots of between 70 and 100 mg were weighed (to 0.0001 g precision) into 13×100 mm glass tubes fitted with Teflon® lined screw caps; the remainder of the powder from each bean was used to determine moisture content, by weight difference after 18 h in a forced air oven at 105° C. Heptane (3 mL) was added to the powders in the tubes and after vortex mixing samples were extracted, on an end-over-end agitator, for 1 h at room temperature. The extracts were centrifuged, 1500×g for 10 min, the supernatant decanted into a clean tube and the pellets were extracted two more times (1 h each) with 1 mL heptane. The supernatants from the three extractions were combined and 50 μL internal standard (triheptadecanoic acid; 10 mg/mL toluene) was added prior to evaporation to dryness at room temperature under a stream of nitrogen gas; standards containing 0, 0.0050, 0.0100, 0.0150, 0.0200 and 0.0300 g soybean oil, in 5 mL heptane, were prepared in the same manner. Fats were converted to fatty acid methyl esters (FAMEs) by adding 1 mL 5% sulfuric acid (v:v. in anhydrous methanol) to the dried pellets and heating them at 80° C. for 30 min, with occasional vortex mixing. The samples were allowed to cool to room temperature and 1 mL 25% aqueous sodium chloride was added followed by 0.8 mL heptane. After vortex mixing the phases were allowed to separate and the upper organic phase was transferred to a sample vial and subjected to GC analysis.

Plotting NMR determined oil contents versus GC determined oil contents resulted in a linear relationship between 9.66 and 26.27% oil (GC values; percent seed wt basis) with a slope of 1.0225 and an R² of 0.9744; based on a seed moisture content that averaged 2.6+/−0.8%.

GC analysis of FAME was employed to investigate if the fatty acid profile of transgenics was altered (increased or decreased) compared to non-transgenic null segregants. Seed were dispensed into individual wells of 96 well strip tubes. For transesterification, 50 μL of trimethylsulfonium hydroxide (TMSH) and 0.5 mL of hexane were added to the each strip tube and incubated for 30 min at room temperature while shaking. Fatty acid methyl esters (1 μL injected from hexane layer) were separated and quantified using a Hewlett-Packard 6890 Gas Chromatograph fitted with an Omegawax 320 fused silica capillary column (Catalog #24152, Supelco Inc.). The oven temperature was programmed to hold at 220° C. for 2.6 min, increase to 240° C. at 20° C./min and then hold for an additional 2.4 min. Carrier gas was supplied by a Whatman hydrogen generator. Retention times were compared to those for methyl esters of standards commercially available (Nu-Chek Prep, Inc.).

### Example 4

### Generation of Soybean Lines with Seed-Targeted Silencing of Galactinol Synthase and Fad3 and Seed Targeted Over-Expression of DGAT Enzymes

Transformation into Soy SSI Target Events

Transgenic SSI target events were produced with the target DNA fragment QC288A as described previously in U.S. Pat. No. 8,293,533 issued Oct. 23, 2012. One target event described in U.S. Pat. No. 8,293,533 issued Oct. 23, 2012, 4729.5.1, also called the “A” line, was chosen to be re-transformed. Target line A contains a well characterized cassette from QC288A having frt1 and frt87 recombination sites with the constitutive SCP1 promoter upstream of the frt1 site.

Suspension cultures were initiated from developing embryos from homozygous plants of target line A using methods described herein.

Target line A cultures were retransformed with the donor construct PHP50573 (SEQ ID NO:19) and the FLP recombinase construct PHP44664 (SEQ ID NO:21) using intact plasmid at a 9:3 pg/bp/prep ratio with the biolistic bombardment transformation protocol described herein and using 90 ng/ml chlorsulfuron (DuPont, Wilmington, Del., USA) as the selection agent. The experiment name given for this transformation was Soil19.

Soil19 events created through RMCE bring the promoter-less als(P178A) coding region of donor construct PHP50573 (SEQ ID NO:19) downstream of the scp1 promoter of QC288A in target line A for expression and thus chlorsulfuron resistance. When the frt1 and frt87 sites from Target line A recombine with those in plasmid PHP70573 in a successful recombination mediated cassette exchange (RMCE), a new 15,646 by DNA sequence is generated in the genomic DNA as set forth in SEQ ID NO:22.

In SEQ ID NO:22, sequence 1-486 is the SCP promoter from Target Line A. Sequence 493-565 is the OMEGA 5′ UTR. Sequence 573-620 is a FLP recombinase recognition site FRT1. Sequence 635-2590 is the soybean acetolactate synthase (als) gene coding region encoding a mutant ALS enzyme insensitive to sulfonylurea herbicides and having a P178A mutation in the encoded protein. Sequence 2607-2917 is the potato proteinase II inhibitor gene (PINII) terminator. Sequence 2953-2968 is a sequence of DNA comprising ORF stop codons in all 6 frames (ORFSTOP-A). Sequence 2971-4140 is the phaseolin transcription terminator. Sequence 4182-4793 is the soy beta-conglycinin promoter. Sequence 4800-6344 is the YLDGAT2 gene. Sequence 6347-7511 is the phaseolin transcription terminator. Sequence 7512-8287 is the soy annexin promoter. Sequence 8294-9252 is the 159-fad3c amiRNA precursor. Sequence 9254-9474 is the soy BD30 transcription terminator. Sequence 9512-11598 is the soy Kunitz Trypsin inhibitor 3 (KTi3) promoter. Sequence 11613-14986 is the GAS123 hairpin. Sequence 14997-15198 is the soy KTi3 transcription terminator. Sequence 9254-9474 is the soy BD30 transcription terminator. Sequence 15202-15483 is the soy albumin transcription terminator. Sequence 15510-11526 is a sequence of DNA comprising ORF stop codons in all 6 frames (ORFSTOP-B). Sequence 15599-15646 is a FLP recombinase recognition site FRT87.

### T0 Embryo and Plant Analysis and Event Selection

Resulting transgenic Soil19 events were selected, maintained and somatic embryos matured as described herein.

Soil19 events were sampled at the somatic embryo stage and screened using construct-specific quantitative PCR (qPCR) as described previously in U.S. Pat. No. 8,293,533 issued Oct. 23, 2012 with oligos designed to check for DNA recombination around the FRT1 site and to check for the presence of target, donor, and Flp DNA. Somatic embryos from those Soil19 events that were positive for correct recombination around the FRT1 site were also analyzed for fatty acid profile using GC-FAME and oil content by NMR on ground embryo powder with methods exactly as described herein. The results for the qPCR, fatty acid and oil analysis of Soil19 events are shown in Table 2. Based on the qPCR, fatty acid composition and oil content data, events were kept as indicated in Table 2. Unless otherwise indicated herein, fatty acids (or respective methyl esters) are always identified as palmitic acid (16:0), stearic acid (18:0), oleic acid (18:1), linoleic acid (18:2) and alpha-linolenic acid (18:3; sometimes referred to as linolenic acid).

TABLE 2
qPCR, fatty acid composition and oil content of embryos from experiment Soil19.

| Soil19 | Fatty Acid Composition | | | | | | | | | | Event |
|---|---|---|---|---|---|---|---|---|---|---|---|
| Event | (wt. %) | | | | | | qPCR Result | | | | Keep |
| (AFS) | 16:0 | 18:0 | 18:1 | 18:2 | 18:3 | % Oil | FRT1 | Donor | Target | FLP | Status |
| 8407-1-1 | 14.9 | 4.1 | 25.3 | 44.8 | 10.8 | 4.8 | 1.44 | 0.00 | 0.00 | 0.00 | Keep |
| 8407-2-1 | 13.2 | 5.2 | 31.4 | 45.3 | 4.9 | 7.0 | 1.43 | 0.00 | 0.00 | 0.00 | Keep |
| 8407-2-2 | 15.3 | 5.2 | 22.4 | 49.0 | 8.1 | 4.7 | 1.28 | 0.00 | 0.30 | 0.00 | Keep |
| 8377-1-1 | 14.1 | 4.5 | 23.2 | 46.6 | 11.5 | 4.9 | 0.29 | 0.14 | 0.86 | 0.00 | Keep |
| 8377-1-2 | 12.9 | 4.8 | 30.1 | 42.7 | 9.4 | 6.4 | 0.86 | 0.00 | 0.00 | 0.00 | Keep |
| 8377-1-3 | 12.6 | 4.7 | 24.7 | 44.3 | 13.7 | 3.8 | 1.62 | 0.00 | 44.87 | 0.00 | Keep |
| 8377-1-4 | 14.1 | 5.0 | 29.4 | 47.1 | 4.4 | 6.8 | 1.60 | 0.75 | 0.64 | 0.00 | Keep |
| 8377-4-6 | 15.4 | 5.8 | 22.8 | 47.0 | 9.0 | 3.1 | 1.85 | 0.00 | 0.45 | 0.00 | Keep |
| 8377-5-1 | 13.7 | 5.7 | 27.9 | 47.2 | 5.4 | 3.0 | 0.00 | 0.62 | 0.00 | 0.96 | Throw |
| 8377-5-2 | 13.7 | 4.1 | 24.0 | 47.8 | 10.4 | 5.1 | 0.00 | 0.00 | 0.36 | 0.00 | Throw |
| 8377-5-3 | 12.6 | 4.5 | 26.2 | 49.4 | 7.4 | 4.9 | 2.14 | 0.00 | 0.78 | 0.00 | Keep |
| 8377-5-4 | 15.4 | 4.4 | 20.4 | 49.9 | 9.9 | 4.0 | 1.74 | 0.00 | 0.75 | 1.23 | Keep |
| 8377-5-5 | 14.0 | 4.8 | 26.9 | 45.4 | 9.0 | 7.3 | 0.00 | 0.50 | 0.85 | 0.00 | Throw |
| 8377-5-6 | 12.2 | 5.0 | 26.5 | 48.6 | 7.7 | 6.7 | 0.00 | 0.48 | 0.00 | 0.00 | Throw |

Somatic embryos from kept Soil19 events were dried, germinated and planted and resulting T0 plants were grown as described herein.

Genomic DNA was isolated from T0 plant leaf tissue, isolated DNA was digested by restriction enzyme, DNA was separated by agarose gel electrophosesis and DNA was blotted and blots were hybridized with suitable ³²P-labeled DNA probes for the hygromycin gene and SCP1 and soy KTi3 promoters in a Southern blot using common methods familiar to those skilled in the art.

In this way, it was determined that T0 plants from kept events AFS 8407-2-2 so and AFS 8377-1-2 contained perfect RMCE insertions into Target Line A and had no additional insertions of PHP50573 (donor) or PHP44664 (flp) DNA in the genome. It was also determined that events AFS 8377-5-3, AFS 8377-4-4 and AFS 8377-1-4 contained perfect RMCE insertions into Target Line A as well as a least one other insertion of donor or flp DNA in the genome. All events were carried to T1 seed.

### T1 Seed Oil Content and Fatty Acid Composition Analysis

Oil content of T1 seed from Soil19 events AFS 8407-2-2 (T1 seed from 4 T0 plants), AFS 8377-1-2 (T1 seed from 1 T0 plant), AFS 8377-5-3 (T1 seed from 1 T0 plant) and AFS 8377-1-4 (T1 seed from 2 T0 plants) was determined by NMR as described herein. A small seed chip was taken from each T1 seed from each event, hexane extracted and the fatty acid composition determined by GC-FAME as described herein. The remaining seed chip was extracted with methanol and soluble sugars separated and visualized by TLC as described in Example 2. T1 seed from plants from Soil19 event AFS 8377-4-4 were not analyzed for phenotypes but were instead planted directly. The results for oil content, fatty acid profile and sugar composition by TLC is shown in Table 3.

In Table 3, oil content is the weight percent oil of total seed weight and the fatty acid profile is the weight percent for individual fatty acids of total fatty acid. The total saturated fatty acids is indicated as Sats and is calculated by summing 16:0 and 18:0 (weight %). In Table 3, the amount of sucrose increase and stachyose decrease as indicated by the TLC plate is qualitatively scored on a scale of 0-3 where a 0 indicates wild-type levels of sugar and a 3 indicates substantially reduced stachyose and substantially increased sucrose. When left blank, the TLC score of a seed chip was not determined. In Table 3, results for each event are divided according to transgenic and null based on the TLC result and the alpha-linolenic content. Results are then sorted based on oil content. The average value for transgenic or null is indicated at the bottom of each column.

TABLE 3
Fatty acid composition, sugar readout and oil
content of T1 Seed from experiment Soil19.

| ¹T1 Seed | | | | | | | | |
|---|---|---|---|---|---|---|---|---|
| (AFS . . .) | TLC | % Oil | 16:0% | 18:0% | 18:1% | 18:2% | 18:3% | Sats |
| 8407-2-2-1-74 | | 29.5 | 9.2 | 5.0 | 30.9 | 53.6 | 1.2 | 14.2 |
| 8407-2-2-1-53 | | 28.4 | 9.2 | 4.4 | 30.6 | 54.1 | 1.8 | 13.6 |
| 8407-2-2-1-15 | 3 | 28.2 | 8.1 | 5.1 | 33.2 | 51.6 | 2.0 | 13.2 |
| 8407-2-2-1-23 | 3 | 27.4 | 9.3 | 4.2 | 29.0 | 55.0 | 2.5 | 13.5 |
| 8407-2-2-1-65 | | 27.4 | 8.8 | 4.8 | 29.3 | 55.0 | 2.0 | 13.6 |
| 8407-2-2-1-45 | 3 | 27.3 | 8.9 | 5.1 | 26.6 | 56.6 | 2.8 | 14.0 |
| 8407-2-2-1-80 | | 27.3 | 9.3 | 4.8 | 25.7 | 56.8 | 3.3 | 14.2 |
| 8407-2-2-1-43 | 3 | 27.3 | 9.1 | 5.6 | 30.9 | 52.6 | 1.9 | 14.6 |
| 8407-2-2-1-36 | 3 | 27.2 | 9.2 | 4.1 | 28.5 | 55.7 | 2.5 | 13.3 |
| 8407-2-2-1-34 | 3 | 27.1 | 8.9 | 5.2 | 28.5 | 54.0 | 3.4 | 14.1 |
| 8407-2-2-1-60 | | 26.8 | 9.3 | 5.3 | 28.5 | 54.8 | 2.0 | 14.7 |
| 8407-2-2-1-51 | | 26.8 | 10.6 | 3.6 | 25.6 | 57.4 | 2.8 | 14.2 |
| 8407-2-2-1-70 | | 26.8 | 9.3 | 4.7 | 26.4 | 56.7 | 2.9 | 14.0 |
| 8407-2-2-1-17 | 3 | 26.7 | 8.8 | 5.8 | 30.7 | 52.4 | 2.3 | 14.6 |
| 8407-2-2-1-63 | | 26.7 | 10.2 | 4.2 | 22.0 | 59.6 | 4.0 | 14.3 |
| 8407-2-2-1-77 | | 26.5 | 9.2 | 5.2 | 26.5 | 57.2 | 1.9 | 14.4 |
| 8407-2-2-1-79 | | 26.4 | 9.0 | 4.7 | 27.9 | 56.3 | 2.1 | 13.7 |
| 8407-2-2-1-31 | 3 | 26.3 | 9.3 | 4.8 | 27.0 | 55.5 | 3.4 | 14.1 |
| 8407-2-2-1-30 | 3 | 26.3 | 8.8 | 4.4 | 25.7 | 56.9 | 4.1 | 13.2 |
| 8407-2-2-1-29 | 3 | 26.3 | 8.5 | 5.5 | 29.8 | 53.8 | 2.4 | 14.1 |
| 8407-2-2-1-72 | | 26.3 | 9.3 | 4.4 | 26.8 | 56.4 | 3.1 | 13.7 |
| 8407-2-2-1-52 | | 26.2 | 9.6 | 4.5 | 24.6 | 58.0 | 3.3 | 14.1 |
| 8407-2-2-1-59 | | 26.1 | 9.5 | 5.0 | 26.0 | 55.9 | 3.6 | 14.5 |
| 8407-2-2-1-56 | | 25.9 | 9.4 | 4.2 | 24.5 | 58.2 | 3.6 | 13.6 |
| 8407-2-2-1-27 | 3 | 25.9 | 8.5 | 4.9 | 27.2 | 55.6 | 3.9 | 13.4 |
| 8407-2-2-1-49 | | 25.9 | 9.6 | 4.6 | 28.4 | 55.5 | 1.9 | 14.2 |
| 8407-2-2-1-57 | | 25.9 | 10.0 | 4.8 | 25.1 | 56.4 | 3.8 | 14.8 |
| 8407-2-2-1-37 | 3 | 25.9 | 9.2 | 4.9 | 26.8 | 55.3 | 3.7 | 14.2 |
| 8407-2-2-1-13 | 3 | 25.9 | 9.3 | 4.6 | 26.5 | 55.8 | 3.8 | 13.9 |
| 8407-2-2-1-21 | 3 | 25.8 | 9.6 | 5.3 | 24.3 | 57.8 | 3.0 | 14.9 |
| 8407-2-2-1-55 | | 25.7 | 9.6 | 4.6 | 25.9 | 56.1 | 3.7 | 14.2 |
| 8407-2-2-1-40 | 3 | 25.7 | 9.3 | 4.4 | 27.7 | 55.5 | 3.1 | 13.7 |
| 8407-2-2-1-8 | 3 | 25.7 | 9.6 | 4.9 | 30.1 | 53.5 | 1.9 | 14.5 |
| 8407-2-2-1-76 | | 25.6 | 9.1 | 4.9 | 30.0 | 54.6 | 1.5 | 13.9 |
| 8407-2-2-1-7 | 3 | 25.6 | 9.0 | 4.4 | 27.0 | 55.7 | 3.9 | 13.3 |
| 8407-2-2-1-5 | 3 | 25.5 | 7.8 | 4.3 | 34.7 | 51.6 | 1.7 | 12.1 |
| 8407-2-2-1-41 | 3 | 25.4 | 8.4 | 5.0 | 32.0 | 52.7 | 1.8 | 13.4 |
| 8407-2-2-1-12 | 3 | 25.4 | 8.6 | 5.1 | 31.8 | 52.3 | 2.1 | 13.8 |
| 8407-2-2-1-67 | | 25.3 | 9.8 | 4.3 | 24.4 | 57.7 | 3.8 | 14.1 |
| 8407-2-2-1-24 | 3 | 25.3 | 8.8 | 5.3 | 27.3 | 55.2 | 3.3 | 14.1 |
| 8407-2-2-1-38 | 3 | 25.2 | 9.8 | 4.5 | 26.2 | 55.8 | 3.7 | 14.3 |
| 8407-2-2-1-71 | | 25.2 | 8.4 | 4.9 | 30.5 | 53.7 | 2.5 | 13.4 |
| 8407-2-2-1-22 | 3 | 25.1 | 9.1 | 4.5 | 26.3 | 56.5 | 3.5 | 13.6 |
| 8407-2-2-1-25 | 3 | 24.9 | 8.8 | 4.5 | 27.6 | 55.9 | 3.2 | 13.3 |
| 8407-2-2-1-26 | 3 | 24.6 | 9.8 | 4.3 | 26.4 | 56.4 | 3.1 | 14.1 |
| 8407-2-2-1-35 | 3 | 24.5 | 8.9 | 4.9 | 28.0 | 54.6 | 3.5 | 13.8 |
| 8407-2-2-1-20 | 3 | 24.4 | 9.6 | 4.6 | 26.6 | 55.2 | 4.0 | 14.2 |
| 8407-2-2-1-46 | 3 | 24.3 | 9.0 | 4.9 | 27.0 | 55.1 | 4.1 | 13.9 |
| 8407-2-2-1-42 | 3 | 24.3 | 9.6 | 4.8 | 26.6 | 55.5 | 3.5 | 14.4 |
| 8407-2-2-1-78 | | 24.2 | 9.0 | 4.5 | 28.7 | 55.4 | 2.4 | 13.5 |
| 8407-2-2-1-3 | 3 | 24.1 | 8.9 | 4.0 | 29.2 | 55.0 | 2.8 | 12.9 |
| 8407-2-2-1-54 | | 23.8 | 10.1 | 3.7 | 26.8 | 55.9 | 3.5 | 13.8 |
| 8407-2-2-1-47 | 3 | 23.8 | 8.9 | 4.0 | 29.8 | 54.3 | 3.0 | 12.9 |
| 8407-2-2-1-6 | 3 | 23.7 | 9.2 | 4.3 | 28.2 | 55.0 | 3.3 | 13.5 |
| 8407-2-2-1-73 | | 23.6 | 10.0 | 4.1 | 27.2 | 55.9 | 2.8 | 14.1 |
| 8407-2-2-1-18 | 3 | 23.6 | 8.5 | 5.3 | 29.9 | 53.8 | 2.4 | 13.8 |
| 8407-2-2-1-10 | 3 | 23.5 | 9.8 | 4.8 | 26.2 | 56.0 | 3.3 | 14.6 |
| 8407-2-2-1-14 | 3 | 23.3 | 9.6 | 4.3 | 28.1 | 55.0 | 3.1 | 13.8 |
| Trans Avg. | | 25.8 | 9.2 | 4.7 | 27.8 | 55.4 | 2.9 | 13.9 |
| 8407-2-2-1-61 | | 24.5 | 11.1 | 4.4 | 16.3 | 57.7 | 10.6 | 15.5 |
| 8407-2-2-1-33 | 0 | 23.7 | 10.2 | 4.2 | 18.3 | 56.5 | 10.7 | 14.4 |
| 8407-2-2-1-44 | 0 | 23.7 | 11.0 | 4.0 | 18.6 | 54.4 | 12.1 | 15.0 |
| 8407-2-2-1-32 | 0 | 23.7 | 11.1 | 4.2 | 19.6 | 53.3 | 11.8 | 15.2 |
| 8407-2-2-1-69 | | 23.3 | 11.9 | 3.7 | 13.7 | 56.2 | 14.5 | 15.6 |
| 8407-2-2-1-11 | 0 | 23.3 | 10.8 | 3.9 | 17.2 | 54.7 | 13.5 | 14.7 |
| 8407-2-2-1-66 | | 23.2 | 11.3 | 3.7 | 15.9 | 57.9 | 11.2 | 15.0 |
| 8407-2-2-1-4 | 0 | 23.1 | 10.9 | 4.2 | 17.8 | 55.2 | 12.0 | 15.0 |
| 8407-2-2-1-50 | | 22.9 | 12.1 | 3.9 | 16.7 | 54.8 | 12.5 | 16.0 |
| 8407-2-2-1-16 | 0 | 22.9 | 11.5 | 3.9 | 17.1 | 54.0 | 13.6 | 15.4 |
| 8407-2-2-1-62 | | 22.9 | 11.6 | 4.1 | 17.1 | 54.0 | 13.2 | 15.7 |
| 8407-2-2-1-64 | | 22.8 | 12.6 | 3.3 | 13.1 | 53.1 | 17.9 | 15.8 |
| 8407-2-2-1-48 | 0 | 22.7 | 10.8 | 3.4 | 21.9 | 52.8 | 11.1 | 14.2 |
| 8407-2-2-1-19 | 0 | 22.6 | 11.1 | 4.3 | 16.3 | 54.5 | 13.8 | 15.4 |
| 8407-2-2-1-2 | 0 | 22.4 | 11.4 | 4.1 | 19.2 | 53.2 | 12.2 | 15.4 |
| 8407-2-2-1-39 | 0 | 21.8 | 10.9 | 3.4 | 18.3 | 55.3 | 12.2 | 14.2 |
| 8407-2-2-1-9 | 0 | 21.6 | 10.8 | 4.1 | 19.9 | 52.7 | 12.5 | 14.9 |
| 8407-2-2-1-58 | | 21.6 | 11.9 | 3.3 | 16.3 | 54.3 | 14.2 | 15.3 |
| 8407-2-2-1-68 | | 21.5 | 13.0 | 0.0 | 12.8 | 55.5 | 18.6 | 13.0 |
| 8407-2-2-1-1 | 0 | 21.0 | 11.1 | 3.5 | 19.8 | 53.2 | 12.4 | 14.5 |
| 8407-2-2-1-28 | 0 | 20.5 | 11.7 | 4.3 | 16.2 | 53.6 | 14.3 | 15.9 |
| 8407-2-2-1-75 | | 20.2 | 12.1 | 3.8 | 16.7 | 53.7 | 13.7 | 15.9 |
| Null Avg. | | 22.5 | 11.4 | 3.7 | 17.2 | 54.6 | 13.1 | 14.3 |
| 8407-2-2-2-20 | 3 | 26.8 | 9.2 | 4.2 | 33.5 | 51.1 | 1.9 | 13.4 |
| 8407-2-2-2-32 | 3 | 26.3 | 8.5 | 4.9 | 31.8 | 51.9 | 2.9 | 13.4 |
| 8407-2-2-2-14 | 3 | 26.1 | 8.6 | 4.2 | 31.9 | 52.4 | 2.9 | 12.8 |
| 8407-2-2-2-48 | 3 | 25.9 | 8.5 | 4.9 | 34.7 | 50.2 | 1.6 | 13.4 |
| 8407-2-2-2-21 | 3 | 25.8 | 8.7 | 5.2 | 31.4 | 52.3 | 2.3 | 13.9 |
| 8407-2-2-2-30 | 3 | 25.7 | 8.9 | 4.3 | 32.8 | 51.7 | 2.3 | 13.2 |
| 8407-2-2-2-35 | 3 | 25.4 | 8.7 | 4.1 | 33.6 | 50.8 | 2.9 | 12.7 |
| 8407-2-2-2-7 | 3 | 25.3 | 8.6 | 4.4 | 28.6 | 55.4 | 2.9 | 13.0 |
| 8407-2-2-2-39 | 3 | 25.2 | 8.9 | 4.9 | 26.4 | 56.1 | 3.7 | 13.8 |
| 8407-2-2-2-3 | 3 | 24.9 | 8.7 | 4.5 | 30.1 | 53.8 | 2.8 | 13.2 |
| 8407-2-2-2-47 | 3 | 24.9 | 8.2 | 5.0 | 38.7 | 46.7 | 1.4 | 13.2 |
| 8407-2-2-2-9 | 3 | 24.5 | 7.8 | 4.9 | 46.7 | 39.4 | 1.3 | 12.7 |
| 8407-2-2-2-40 | 3 | 24.5 | 9.5 | 4.3 | 28.4 | 54.8 | 3.0 | 13.8 |
| 8407-2-2-2-12 | 3 | 24.5 | 8.9 | 5.9 | 26.0 | 55.7 | 3.6 | 14.8 |
| 8407-2-2-2-18 | 3 | 24.5 | 8.6 | 5.3 | 29.8 | 54.2 | 2.1 | 13.9 |
| 8407-2-2-2-38 | 3 | 24.5 | 9.1 | 4.2 | 31.9 | 52.5 | 2.3 | 13.2 |
| 8407-2-2-2-25 | 3 | 24.0 | 8.6 | 4.6 | 30.3 | 53.3 | 3.1 | 13.2 |
| 8407-2-2-2-45 | 3 | 24.0 | 8.5 | 5.2 | 39.1 | 45.7 | 1.5 | 13.7 |
| 8407-2-2-2-41 | 3 | 23.9 | 8.0 | 4.5 | 40.0 | 46.1 | 1.4 | 12.5 |
| 8407-2-2-2-42 | 3 | 23.7 | 8.1 | 4.6 | 37.7 | 47.8 | 1.8 | 12.7 |
| 8407-2-2-2-27 | 3 | 23.5 | 9.2 | 4.8 | 36.3 | 48.2 | 1.6 | 14.0 |
| 8407-2-2-2-31 | 3 | 23.5 | 7.6 | 4.6 | 35.0 | 50.6 | 2.3 | 12.2 |
| 8407-2-2-2-23 | 3 | 23.4 | 8.7 | 4.2 | 35.7 | 48.9 | 2.5 | 12.9 |
| 8407-2-2-2-22 | 3 | 23.4 | 8.9 | 4.7 | 33.1 | 50.4 | 2.8 | 13.6 |
| 8407-2-2-2-43 | 3 | 23.3 | 8.0 | 4.4 | 37.2 | 48.9 | 1.5 | 12.5 |
| 8407-2-2-2-2 | 3 | 23.3 | 9.0 | 4.6 | 29.4 | 53.8 | 3.2 | 13.6 |
| 8407-2-2-2-19 | 3 | 23.2 | 8.7 | 3.7 | 38.0 | 47.6 | 2.0 | 12.4 |
| 8407-2-2-2-1 | 3 | 23.0 | 8.5 | 5.1 | 36.4 | 48.5 | 1.6 | 13.6 |
| 8407-2-2-2-11 | 3 | 22.9 | 8.8 | 4.2 | 34.8 | 50.5 | 1.7 | 13.0 |
| 8407-2-2-2-37 | 3 | 22.6 | 8.9 | 5.2 | 32.1 | 51.9 | 1.9 | 14.1 |
| 8407-2-2-2-10 | 3 | 22.1 | 7.9 | 4.0 | 39.9 | 46.1 | 2.1 | 11.8 |
| 8407-2-2-2-8 | 3 | 22.0 | 8.9 | 4.2 | 34.7 | 49.6 | 2.7 | 13.1 |
| 8407-2-2-2-36 | 3 | 21.8 | 8.5 | 5.6 | 33.8 | 48.9 | 3.1 | 14.2 |
| 8407-2-2-2-5 | 3 | 21.6 | 9.6 | 4.5 | 24.9 | 57.7 | 3.4 | 14.1 |
| 8407-2-2-2-34 | 3 | 17.0 | 8.5 | 4.5 | 53.0 | 31.9 | 2.1 | 13.0 |
| Trans Avg. | | 23.9 | 8.6 | 4.6 | 34.2 | 50.2 | 2.3 | 13.3 |
| 8407-2-2-2-29 | 0 | 22.8 | 10.3 | 3.5 | 24.0 | 51.8 | 10.4 | 13.8 |
| 8407-2-2-2-28 | 0 | 22.2 | 10.6 | 4.2 | 20.5 | 54.3 | 10.5 | 14.8 |
| 8407-2-2-2-16 | 0 | 18.9 | 10.8 | 3.2 | 25.2 | 50.2 | 10.6 | 13.9 |
| 8407-2-2-2-13 | 0 | 21.6 | 10.7 | 3.8 | 21.9 | 52.9 | 10.7 | 14.5 |
| 8407-2-2-2-24 | 0 | 20.4 | 11.3 | 3.4 | 23.6 | 50.5 | 11.1 | 14.7 |
| 8407-2-2-2-44 | 0 | 21.1 | 10.1 | 3.9 | 19.4 | 55.3 | 11.3 | 14.0 |
| 8407-2-2-2-33 | 0 | 15.1 | 10.7 | 2.4 | 28.6 | 46.5 | 11.7 | 13.1 |
| 8407-2-2-2-15 | 0 | 20.6 | 10.4 | 3.6 | 23.2 | 50.9 | 11.9 | 14.1 |
| 8407-2-2-2-26 | 0 | 19.5 | 11.1 | 3.6 | 22.4 | 50.7 | 12.2 | 14.7 |
| 8407-2-2-2-46 | 0 | 23.5 | 10.7 | 4.4 | 19.2 | 53.4 | 12.3 | 15.1 |
| 8407-2-2-2-4 | 0 | 21.0 | 10.7 | 3.3 | 21.7 | 51.8 | 12.5 | 14.0 |
| 8407-2-2-2-6 | 0 | 22.6 | 10.7 | 3.7 | 21.0 | 50.9 | 13.7 | 14.4 |
| 8407-2-2-2-17 | 0 | 21.1 | 10.9 | 3.7 | 15.9 | 55.1 | 14.3 | 14.6 |
| Null Avg. | | 20.8 | 10.7 | 3.6 | 22.1 | 51.9 | 11.8 | 14.3 |
| 8407-2-2-3-10 | 3 | 27.7 | 9.4 | 5.8 | 26.2 | 55.2 | 3.4 | 15.2 |
| 8407-2-2-3-26 | 3 | 26.7 | 8.8 | 4.5 | 30.5 | 52.9 | 3.4 | 13.3 |
| 8407-2-2-3-7 | 3 | 26.6 | 9.1 | 6.1 | 27.2 | 54.7 | 3.0 | 15.2 |
| 8407-2-2-3-8 | 3 | 24.9 | 8.1 | 4.5 | 31.4 | 53.9 | 2.2 | 12.5 |
| 8407-2-2-3-22 | 3 | 24.5 | 8.9 | 5.0 | 34.2 | 50.2 | 1.7 | 13.9 |
| 8407-2-2-3-47 | 3 | 23.8 | 8.8 | 4.6 | 29.7 | 54.2 | 2.7 | 13.4 |
| 8407-2-2-3-13 | 3 | 23.8 | 8.3 | 4.5 | 36.7 | 48.9 | 1.6 | 12.8 |
| 8407-2-2-3-2 | 3 | 23.7 | 8.7 | 4.1 | 30.3 | 53.3 | 3.6 | 12.8 |
| 8407-2-2-3-25 | 3 | 23.4 | 8.7 | 5.0 | 33.1 | 51.3 | 1.9 | 13.7 |
| 8407-2-2-3-43 | 3 | 23.3 | 8.0 | 4.8 | 34.3 | 50.8 | 2.1 | 12.8 |
| 8407-2-2-3-3 | 3 | 23.1 | 8.6 | 4.5 | 31.1 | 53.6 | 2.2 | 13.1 |
| 8407-2-2-3-18 | 3 | 23.0 | 9.4 | 4.8 | 26.4 | 55.6 | 3.8 | 14.2 |
| 8407-2-2-3-16 | 3 | 23.0 | 9.7 | 4.7 | 25.6 | 56.2 | 3.8 | 14.4 |
| 8407-2-2-3-1 | 3 | 22.9 | 9.3 | 4.6 | 28.1 | 55.3 | 2.8 | 13.8 |
| 8407-2-2-3-6 | 3 | 22.9 | 9.1 | 4.5 | 28.8 | 54.0 | 3.6 | 13.6 |
| 8407-2-2-3-40 | 3 | 22.8 | 8.7 | 4.9 | 32.8 | 51.9 | 1.7 | 13.6 |
| 8407-2-2-3-11 | 3 | 22.7 | 9.1 | 4.5 | 28.0 | 55.4 | 3.0 | 13.6 |
| 8407-2-2-3-23 | 3 | 22.6 | 8.0 | 5.0 | 34.2 | 51.1 | 1.7 | 13.0 |
| 8407-2-2-3-14 | 3 | 22.5 | 9.9 | 4.7 | 24.7 | 57.3 | 3.4 | 14.5 |
| 8407-2-2-3-36 | 3 | 22.3 | 10.1 | 4.8 | 21.5 | 58.8 | 4.8 | 14.9 |
| 8407-2-2-3-46 | 3 | 22.3 | 9.1 | 4.4 | 25.9 | 57.2 | 3.5 | 13.5 |
| 8407-2-2-3-30 | 3 | 22.2 | 8.2 | 5.6 | 33.3 | 50.8 | 2.1 | 13.8 |
| 8407-2-2-3-41 | 2 | 22.1 | 9.4 | 4.7 | 28.2 | 54.7 | 3.0 | 14.1 |
| 8407-2-2-3-42 | 2 | 22.0 | 9.0 | 4.1 | 29.2 | 54.6 | 3.1 | 13.1 |
| 8407-2-2-3-44 | 3 | 21.7 | 9.5 | 4.1 | 26.1 | 57.1 | 3.2 | 13.6 |
| 8407-2-2-3-34 | 3 | 21.6 | 9.2 | 4.6 | 26.6 | 56.6 | 3.0 | 13.8 |
| 8407-2-2-3-32 | 3 | 21.3 | 10.2 | 4.0 | 28.6 | 54.4 | 2.8 | 14.2 |
| 8407-2-2-3-35 | 3 | 21.1 | 9.1 | 4.2 | 27.2 | 56.6 | 2.9 | 13.3 |
| 8407-2-2-3-15 | 3 | 20.7 | 8.9 | 4.3 | 27.4 | 56.7 | 2.6 | 13.2 |
| 8407-2-2-3-29 | 3 | 20.7 | 8.6 | 5.0 | 29.0 | 53.7 | 3.6 | 13.6 |
| 8407-2-2-3-38 | 3 | 20.5 | 10.3 | 4.8 | 26.3 | 55.7 | 2.8 | 15.2 |
| Trans Avg. | | 23.0 | 9.0 | 4.7 | 29.1 | 54.3 | 2.9 | 13.7 |
| 8407-2-2-3-31 | 0 | 21.0 | 10.0 | 4.1 | 18.3 | 56.6 | 11.1 | 14.0 |
| 8407-2-2-3-37 | 0 | 20.9 | 9.8 | 3.6 | 16.6 | 55.6 | 14.3 | 13.4 |
| 8407-2-2-3-48 | 0 | 20.7 | 11.3 | 3.7 | 17.1 | 54.6 | 13.2 | 15.0 |
| 8407-2-2-3-39 | 0 | 20.7 | 11.7 | 3.6 | 20.7 | 52.3 | 11.8 | 15.3 |
| 8407-2-2-3-20 | 0 | 20.4 | 11.1 | 3.9 | 17.0 | 53.9 | 14.1 | 15.0 |
| 8407-2-2-3-28 | 0 | 20.4 | 10.6 | 4.0 | 15.3 | 55.3 | 14.8 | 14.6 |
| 8407-2-2-3-21 | 0 | 20.4 | 10.6 | 4.1 | 17.0 | 54.0 | 14.3 | 14.7 |
| 8407-2-2-3-33 | 0 | 20.2 | 10.7 | 3.4 | 18.4 | 55.1 | 12.4 | 14.1 |
| 8407-2-2-3-45 | 0 | 19.8 | 10.3 | 3.4 | 19.8 | 53.4 | 13.2 | 13.7 |
| 8407-2-2-3-5 | 0 | 19.5 | 11.1 | 3.4 | 17.1 | 54.1 | 14.3 | 14.5 |
| 8407-2-2-3-12 | 0 | 19.4 | 10.8 | 3.3 | 22.5 | 52.0 | 11.4 | 14.0 |
| 8407-2-2-3-27 | 0 | 19.3 | 9.9 | 3.7 | 17.4 | 57.2 | 11.9 | 13.5 |
| 8407-2-2-3-24 | 0 | 19.2 | 11.1 | 3.8 | 19.0 | 52.8 | 13.3 | 14.9 |
| 8407-2-2-3-19 | 0 | 19.0 | 11.4 | 3.4 | 23.3 | 51.5 | 10.4 | 14.8 |
| 8407-2-2-3-17 | 0 | 18.7 | 11.5 | 3.6 | 14.0 | 55.6 | 15.3 | 15.2 |
| 8407-2-2-3-9 | 0 | 18.1 | 10.5 | 3.4 | 13.2 | 54.7 | 18.2 | 13.9 |
| 8407-2-2-3-4 | 0 | 17.1 | 11.1 | 3.3 | 17.3 | 53.7 | 14.7 | 14.3 |
| Null Avg. | | 19.7 | 10.8 | 3.6 | 17.9 | 54.3 | 13.5 | 14.4 |
| 8407-2-2-4-24 | 3 | 27.9 | 8.5 | 5.8 | 31.9 | 51.6 | 2.2 | 14.3 |
| 8407-2-2-4-43 | 3 | 27.9 | 7.8 | 6.0 | 29.0 | 54.7 | 2.5 | 13.7 |
| 8407-2-2-4-15 | 3 | 27.1 | 7.9 | 5.4 | 33.4 | 51.0 | 2.3 | 13.3 |
| 8407-2-2-4-6 | 3 | 27.1 | 8.3 | 5.5 | 32.2 | 51.8 | 2.2 | 13.8 |
| 8407-2-2-4-1 | 3 | 27.0 | 9.2 | 5.2 | 27.2 | 55.6 | 2.9 | 14.4 |
| 8407-2-2-4-16 | 3 | 26.6 | 9.0 | 5.2 | 29.2 | 53.6 | 3.1 | 14.2 |
| 8407-2-2-4-48 | 3 | 26.4 | 8.1 | 5.2 | 32.1 | 52.4 | 2.1 | 13.4 |
| 8407-2-2-4-8 | 3 | 26.4 | 8.3 | 5.1 | 32.4 | 52.3 | 1.9 | 13.4 |
| 8407-2-2-4-10 | 3 | 26.3 | 8.9 | 4.8 | 28.8 | 54.6 | 3.0 | 13.6 |
| 8407-2-2-4-37 | 3 | 26.0 | 8.9 | 5.4 | 32.6 | 51.0 | 2.1 | 14.3 |
| 8407-2-2-4-34 | 3 | 26.0 | 9.0 | 4.5 | 29.7 | 53.9 | 3.0 | 13.4 |
| 8407-2-2-4-13 | 3 | 26.0 | 8.9 | 5.4 | 28.1 | 54.4 | 3.2 | 14.3 |
| 8407-2-2-4-20 | 3 | 25.9 | 9.0 | 5.6 | 26.1 | 56.3 | 3.1 | 14.5 |
| 8407-2-2-4-35 | 3 | 25.8 | 9.0 | 5.8 | 23.4 | 57.3 | 4.4 | 14.8 |
| 8407-2-2-4-38 | 3 | 25.8 | 8.8 | 4.7 | 30.3 | 52.3 | 3.8 | 13.5 |
| 8407-2-2-4-28 | 3 | 25.7 | 9.2 | 5.2 | 27.5 | 55.1 | 3.0 | 14.3 |
| 8407-2-2-4-31 | 3 | 25.7 | 9.1 | 5.7 | 27.0 | 54.7 | 3.5 | 14.8 |
| 8407-2-2-4-18 | 3 | 25.6 | 9.0 | 5.2 | 31.8 | 51.5 | 2.5 | 14.2 |
| 8407-2-2-4-17 | 3 | 25.6 | 9.0 | 5.0 | 27.6 | 55.3 | 3.1 | 14.0 |
| 8407-2-2-4-5 | 3 | 25.5 | 9.1 | 5.8 | 22.3 | 58.2 | 4.6 | 14.9 |
| 8407-2-2-4-19 | 3 | 25.4 | 0.0 | 0.0 | 36.9 | 63.1 | 0.0 | 0.0 |
| 8407-2-2-4-23 | 3 | 25.3 | 8.9 | 5.6 | 31.1 | 52.3 | 2.1 | 14.5 |
| 8407-2-2-4-7 | 3 | 25.2 | 8.5 | 5.7 | 30.8 | 52.8 | 2.3 | 14.2 |
| 8407-2-2-4-44 | 3 | 24.9 | 9.0 | 4.9 | 29.2 | 53.4 | 3.4 | 13.9 |
| 8407-2-2-4-46 | 3 | 24.8 | 9.5 | 4.8 | 26.9 | 54.8 | 4.0 | 14.3 |
| 8407-2-2-4-21 | 3 | 24.7 | 9.1 | 5.1 | 25.1 | 56.4 | 4.3 | 14.2 |
| 8407-2-2-4-4 | 3 | 24.7 | 9.0 | 4.3 | 32.4 | 52.4 | 1.8 | 13.3 |
| 8407-2-2-4-3 | 3 | 24.6 | 8.3 | 4.6 | 35.4 | 50.3 | 1.5 | 12.9 |
| 8407-2-2-4-47 | 3 | 24.5 | 9.1 | 4.3 | 29.0 | 54.2 | 3.3 | 13.5 |
| 8407-2-2-4-36 | 3 | 24.4 | 9.4 | 4.7 | 27.0 | 55.1 | 3.9 | 14.1 |
| 8407-2-2-4-27 | 3 | 24.2 | 9.1 | 5.1 | 24.5 | 56.0 | 5.3 | 14.2 |
| 8407-2-2-4-33 | 3 | 23.7 | 9.3 | 4.3 | 28.4 | 54.7 | 3.3 | 13.6 |
| 8407-2-2-4-40 | 3 | 23.5 | 9.5 | 4.5 | 26.2 | 55.7 | 4.1 | 14.0 |
| 8407-2-2-4-2 | 3 | 23.5 | 9.3 | 4.4 | 26.6 | 54.8 | 4.9 | 13.7 |
| 8407-2-2-4-30 | 3 | 22.3 | 8.3 | 6.1 | 32.6 | 50.6 | 2.5 | 14.4 |
| 8407-2-2-4-11 | 0 | 22.2 | 9.0 | 4.7 | 30.4 | 51.3 | 4.6 | 13.7 |
| Trans Avg. | | 25.4 | 8.6 | 5.0 | 29.3 | 54.0 | 3.1 | 13.6 |
| 8407-2-2-4-9 | 0 | 24.7 | 10.4 | 5.2 | 19.1 | 54.3 | 11.1 | 11.1 |
| 8407-2-2-4-39 | 0 | 24.5 | 10.8 | 4.1 | 16.5 | 55.3 | 13.3 | 14.8 |
| 8407-2-2-4-45 | 0 | 23.6 | 10.9 | 4.2 | 16.6 | 56.3 | 12.0 | 15.1 |
| 8407-2-2-4-12 | 0 | 23.0 | 10.8 | 4.1 | 17.4 | 53.8 | 13.9 | 14.8 |
| 8407-2-2-4-22 | 0 | 22.9 | 10.7 | 3.9 | 20.7 | 53.6 | 11.0 | 14.7 |
| 8407-2-2-4-14 | 0 | 22.9 | 11.0 | 3.7 | 18.9 | 53.0 | 13.3 | 14.8 |
| 8407-2-2-4-32 | 0 | 22.7 | 10.9 | 4.2 | 18.7 | 53.6 | 12.7 | 15.0 |
| 8407-2-2-4-25 | 0 | 22.7 | 10.9 | 4.4 | 18.2 | 53.7 | 12.8 | 15.3 |
| 8407-2-2-4-42 | 0 | 22.4 | 11.4 | 4.6 | 20.8 | 53.1 | 10.1 | 16.0 |
| 8407-2-2-4-29 | 0 | 21.7 | 11.6 | 4.7 | 17.8 | 53.2 | 12.7 | 16.3 |
| 8407-2-2-4-26 | 0 | 21.2 | 11.0 | 3.6 | 15.2 | 53.6 | 16.6 | 14.6 |
| 8407-2-2-4-41 | 0 | 20.9 | 11.8 | 3.7 | 17.6 | 54.7 | 12.3 | 15.4 |
| Null Avg. | | 22.8 | 11.0 | 4.2 | 18.1 | 54.0 | 12.6 | 14.8 |
| 8377-1-4-2-24 | 3 | 25.8 | 9.2 | 4.8 | 27.0 | 55.3 | 3.7 | 14.0 |
| 8377-1-4-2-42 | 3 | 25.7 | 9.4 | 5.3 | 26.7 | 56.4 | 2.2 | 14.7 |
| 8377-1-4-2-41 | 3 | 25.3 | 9.3 | 4.3 | 28.5 | 55.9 | 1.9 | 13.6 |
| 8377-1-4-2-2 | 3 | 25.2 | 8.5 | 4.3 | 33.0 | 51.9 | 2.2 | 12.8 |
| 8377-1-4-2-19 | 3 | 24.9 | 8.1 | 5.9 | 34.9 | 49.6 | 1.5 | 14.0 |
| 8377-1-4-2-21 | 3 | 24.9 | 8.9 | 5.1 | 27.4 | 56.5 | 2.1 | 14.0 |
| 8377-1-4-2-45 | 3 | 24.8 | 8.1 | 5.0 | 33.8 | 51.7 | 1.4 | 13.1 |
| 8377-1-4-2-3 | 3 | 24.8 | 7.9 | 5.6 | 35.1 | 50.0 | 1.4 | 13.5 |
| 8377-1-4-2-22 | 3 | 24.4 | 9.0 | 6.2 | 29.0 | 54.0 | 1.8 | 15.3 |
| 8377-1-4-2-1 | 3 | 24.3 | 8.6 | 4.3 | 28.3 | 54.5 | 4.3 | 12.9 |
| 8377-1-4-2-34 | 3 | 24.3 | 9.1 | 5.1 | 27.1 | 55.8 | 2.9 | 14.3 |
| 8377-1-4-2-14 | 3 | 24.3 | 8.7 | 4.8 | 27.6 | 55.9 | 3.0 | 13.5 |
| 8377-1-4-2-33 | 3 | 24.2 | 8.1 | 4.4 | 35.1 | 50.7 | 1.6 | 12.5 |
| 8377-1-4-2-39 | 3 | 24.0 | 9.1 | 3.4 | 29.5 | 56.2 | 1.9 | 12.5 |
| 8377-1-4-2-29 | 3 | 24.0 | 8.8 | 4.4 | 28.5 | 56.5 | 1.7 | 13.2 |
| 8377-1-4-2-38 | 3 | 23.9 | 8.9 | 5.8 | 28.3 | 55.1 | 1.9 | 14.7 |
| 8377-1-4-2-10 | 3 | 23.7 | 8.8 | 6.5 | 33.5 | 49.8 | 1.4 | 15.3 |
| 8377-1-4-2-11 | 3 | 23.7 | 8.3 | 4.9 | 35.9 | 49.3 | 1.6 | 13.2 |
| 8377-1-4-2-26 | 3 | 23.6 | 8.7 | 5.5 | 28.1 | 53.9 | 3.8 | 14.2 |
| 8377-1-4-2-16 | 3 | 23.6 | 8.9 | 4.4 | 29.9 | 55.1 | 1.8 | 13.2 |
| 8377-1-4-2-8 | 3 | 23.6 | 9.1 | 4.4 | 29.2 | 55.5 | 1.8 | 13.5 |
| 8377-1-4-2-13 | 3 | 23.5 | 8.9 | 4.2 | 32.6 | 51.5 | 2.8 | 13.1 |
| 8377-1-4-2-43 | 3 | 23.4 | 8.1 | 5.2 | 36.4 | 49.1 | 1.2 | 13.3 |
| 8377-1-4-2-27 | 3 | 23.2 | 9.3 | 4.5 | 27.7 | 56.6 | 1.9 | 13.8 |
| 8377-1-4-2-32 | 3 | 23.0 | 7.7 | 4.9 | 38.7 | 46.6 | 2.1 | 12.6 |
| 8377-1-4-2-44 | 3 | 22.9 | 9.2 | 5.3 | 28.9 | 54.5 | 2.1 | 14.5 |
| 8377-1-4-2-46 | 3 | 22.9 | 8.7 | 4.9 | 31.1 | 53.4 | 1.9 | 13.7 |
| 8377-1-4-2-40 | 3 | 22.9 | 9.1 | 4.8 | 31.4 | 52.8 | 1.8 | 13.9 |
| 8377-1-4-2-17 | 3 | 22.9 | 9.0 | 5.3 | 27.9 | 55.5 | 2.3 | 14.3 |
| 8377-1-4-2-18 | 3 | 22.7 | 9.0 | 5.3 | 30.5 | 53.2 | 1.9 | 14.3 |
| 8377-1-4-2-6 | 3 | 22.7 | 8.9 | 5.5 | 32.1 | 51.3 | 2.2 | 14.4 |
| 8377-1-4-2-30 | 3 | 22.2 | 8.9 | 4.5 | 29.5 | 53.6 | 3.5 | 13.4 |
| 8377-1-4-2-37 | 3 | 22.1 | 8.4 | 5.5 | 33.1 | 50.9 | 2.1 | 13.8 |
| 8377-1-4-2-7 | 3 | 22.1 | 8.5 | 4.2 | 31.2 | 53.8 | 2.2 | 12.8 |
| 8377-1-4-2-36 | 3 | 22.0 | 9.0 | 4.9 | 30.4 | 53.7 | 2.1 | 13.9 |
| 8377-1-4-2-20 | 3 | 21.9 | 10.7 | 4.0 | 20.7 | 60.6 | 4.0 | 14.7 |
| 8377-1-4-2-48 | 3 | 21.5 | 10.7 | 3.7 | 18.5 | 63.8 | 3.2 | 14.4 |
| 8377-1-4-2-15 | 3 | 21.5 | 10.2 | 4.0 | 18.5 | 64.4 | 2.9 | 14.2 |
| 8377-1-4-2-23 | 3 | 21.2 | 11.5 | 2.0 | 13.1 | 68.8 | 4.6 | 13.5 |
| 8377-1-4-2-4 | 3 | 21.1 | 10.6 | 4.1 | 16.3 | 64.6 | 4.4 | 14.7 |
| 8377-1-4-2-12 | 3 | 20.9 | 10.5 | 4.0 | 20.3 | 61.4 | 3.9 | 14.5 |
| 8377-1-4-2-47 | 3 | 20.7 | 10.7 | 4.0 | 17.6 | 64.7 | 3.1 | 14.7 |
| 8377-1-4-2-31 | 3 | 20.7 | 10.7 | 3.3 | 20.9 | 61.9 | 3.3 | 14.0 |
| 8377-1-4-2-25 | 3 | 20.6 | 9.7 | 3.7 | 19.8 | 63.4 | 3.4 | 13.5 |
| 8377-1-4-2-9 | 3 | 20.1 | 10.6 | 3.5 | 20.5 | 61.4 | 4.0 | 14.1 |
| 8377-1-4-2-35 | 3 | 19.7 | 10.1 | 5.0 | 24.7 | 58.5 | 1.7 | 15.0 |
| Trans Avg. | | 23.1 | 9.2 | 4.7 | 28.0 | 55.6 | 2.5 | 13.9 |
| 8377-1-4-2-5 | 0 | 21.7 | 10.2 | 3.8 | 16.2 | 55.4 | 14.4 | 14.0 |
| 8377-1-4-2-28 | 0 | 21.3 | 10.6 | 3.7 | 17.1 | 55.2 | 13.4 | 14.3 |
| Null Avg. | | 21.5 | 10.4 | 3.8 | 16.6 | 55.3 | 13.9 | 14.1 |
| 8377-1-4-3-32 | 3 | 24.9 | 8.1 | 7.4 | 36.1 | 46.7 | 1.6 | |
| 8377-1-4-3-23 | 3 | 24.6 | 7.9 | 8.8 | 35.3 | 46.7 | 1.2 | 16.7 |
| 8377-1-4-3-37 | 3 | 24.5 | 8.3 | 6.2 | 36.6 | 47.2 | 1.7 | 14.5 |
| 8377-1-4-3-48 | 3 | 23.8 | 8.9 | 5.2 | 27.4 | 54.2 | 4.4 | 14.1 |
| 8377-1-4-3-45 | 3 | 23.8 | 7.7 | 5.9 | 40.2 | 44.0 | 2.3 | 13.6 |
| 8377-1-4-3-30 | 3 | 23.3 | 8.7 | 5.3 | 27.7 | 54.2 | 4.1 | 14.1 |
| 8377-1-4-3-19 | 3 | 22.5 | 8.9 | 4.4 | 27.0 | 55.0 | 4.8 | 13.2 |
| 8377-1-4-3-18 | 3 | 22.3 | 8.3 | 7.3 | 38.1 | 44.9 | 1.5 | 15.5 |
| 8377-1-4-3-17 | 3 | 22.3 | 8.6 | 4.4 | 29.2 | 53.5 | 4.3 | 13.0 |
| 8377-1-4-3-2 | 3 | 22.1 | 8.2 | 5.3 | 37.0 | 47.3 | 2.2 | 13.5 |
| 8377-1-4-3-31 | 3 | 21.9 | 8.9 | 5.6 | 36.9 | 47.2 | 1.4 | 14.5 |
| 8377-1-4-3-12 | 3 | 21.7 | 8.5 | 4.4 | 33.8 | 48.7 | 4.6 | 12.9 |
| 8377-1-4-3-38 | 3 | 21.5 | 9.0 | 4.4 | 30.9 | 51.6 | 4.1 | 13.4 |
| 8377-1-4-3-4 | 3 | 21.4 | 9.4 | 5.6 | 25.1 | 53.8 | 6.2 | 15.0 |
| 8377-1-4-3-14 | 3 | 21.2 | 8.5 | 4.1 | 29.7 | 53.0 | 4.7 | 12.6 |
| 8377-1-4-3-11 | 3 | 21.1 | 8.1 | 5.9 | 40.1 | 44.4 | 1.5 | 14.0 |
| 8377-1-4-3-43 | 3 | 21.1 | 8.2 | 4.8 | 44.0 | 41.3 | 1.7 | 13.0 |
| 8377-1-4-3-22 | 3 | 21.1 | 8.7 | 4.0 | 33.5 | 50.0 | 3.7 | 12.7 |
| 8377-1-4-3-33 | 3 | 21.1 | 7.2 | 4.1 | 45.3 | 41.5 | 1.9 | 11.3 |
| 8377-1-4-3-6 | 3 | 21.0 | 8.8 | 4.0 | 32.9 | 49.6 | 4.7 | 12.8 |
| 8377-1-4-3-39 | 3 | 20.8 | 8.7 | 4.1 | 28.1 | 54.1 | 5.0 | 12.8 |
| 8377-1-4-3-25 | 3 | 20.7 | 8.0 | 4.1 | 32.7 | 51.0 | 4.3 | 12.1 |
| 8377-1-4-3-16 | 3 | 20.6 | 8.8 | 4.0 | 30.8 | 52.4 | 4.1 | 12.8 |
| 8377-1-4-3-9 | 3 | 20.4 | 7.7 | 5.1 | 43.5 | 42.1 | 1.6 | 12.8 |
| 8377-1-4-3-29 | 3 | 20.3 | 9.2 | 4.9 | 24.2 | 57.1 | 4.6 | 14.1 |
| 8377-1-4-3-40 | 3 | 20.2 | 7.5 | 4.9 | 41.9 | 43.8 | 2.0 | 12.4 |
| 8377-1-4-3-41 | 3 | 20.0 | 6.5 | 4.6 | 44.5 | 42.5 | 1.8 | 11.1 |
| 8377-1-4-3-36 | 3 | 19.9 | 8.7 | 5.4 | 29.4 | 52.9 | 3.6 | 14.1 |
| 8377-1-4-3-26 | 3 | 19.2 | 9.0 | 5.6 | 38.3 | 45.3 | 1.8 | 14.6 |
| 8377-1-4-3-20 | 3 | 19.0 | 8.2 | 4.7 | 38.4 | 46.1 | 2.6 | 12.9 |
| 8377-1-4-3-15 | 3 | 18.8 | 7.3 | 4.4 | 41.5 | 45.0 | 1.9 | 11.7 |
| 8377-1-4-3-28 | 3 | 18.7 | 7.0 | 5.4 | 46.9 | 39.1 | 1.5 | 12.5 |
| 8377-1-4-3-42 | 3 | 18.6 | 7.7 | 5.4 | 40.0 | 45.3 | 1.7 | 13.1 |
| 8377-1-4-3-8 | 3 | 18.5 | 7.1 | 3.6 | 46.2 | 41.2 | 1.9 | 10.7 |
| 8377-1-4-3-44 | 3 | 18.1 | 8.3 | 5.1 | 38.3 | 46.4 | 1.8 | 13.4 |
| 8377-1-4-3-24 | 3 | 17.9 | 7.8 | 4.2 | 38.9 | 47.1 | 2.0 | 12.0 |
| 8377-1-4-3-34 | 3 | 17.4 | 7.6 | 4.5 | 43.6 | 42.1 | 2.1 | 12.2 |
| 8377-1-4-3-3 | 3 | 17.0 | 7.9 | 5.5 | 38.4 | 46.0 | 2.1 | 13.4 |
| Trans Avg. | | 20.9 | 8.2 | 5.1 | 36.1 | 47.7 | 2.9 | 13.2 |
| 8377-1-4-3-21 | 0 | 20.6 | 9.8 | 4.1 | 18.6 | 54.7 | 12.8 | 13.9 |
| 8377-1-4-3-10 | 0 | 19.4 | 10.1 | 3.7 | 18.9 | 51.8 | 15.5 | 13.8 |
| 8377-1-4-3-47 | 0 | 19.1 | 9.8 | 3.5 | 19.7 | 54.1 | 13.0 | 13.3 |
| 8377-1-4-3-1 | 0 | 18.9 | 10.0 | 3.4 | 20.9 | 52.1 | 13.6 | 13.4 |
| 8377-1-4-3-7 | 0 | 18.8 | 9.0 | 3.3 | 18.6 | 54.7 | 14.3 | 12.3 |
| 8377-1-4-3-13 | 0 | 18.6 | 9.7 | 3.1 | 21.8 | 51.0 | 14.4 | 12.7 |
| 8377-1-4-3-5 | 0 | 18.4 | 9.8 | 3.6 | 17.9 | 53.8 | 14.9 | 13.3 |
| 8377-1-4-3-27 | 0 | 18.0 | 9.7 | 3.4 | 18.9 | 52.1 | 16.0 | 13.1 |
| 8377-1-4-3-46 | 0 | 17.5 | 10.1 | 3.2 | 19.4 | 50.8 | 16.5 | 13.3 |
| 8377-1-4-3-35 | 0 | 16.4 | 10.0 | 3.4 | 23.0 | 51.0 | 12.5 | 13.5 |
| Null Avg. | | 18.6 | 9.8 | 3.5 | 19.8 | 52.6 | 14.4 | 13.3 |
| 8377-5-3-3-16 | 3 | 22.4 | 9.0 | 3.6 | 31.8 | 52.3 | 3.4 | 12.6 |
| 8377-5-3-3-33 | 3 | 22.1 | 9.7 | 4.1 | 36.5 | 47.1 | 2.5 | 13.8 |
| 8377-5-3-3-30 | 3 | 21.9 | 9.3 | 4.3 | 31.0 | 53.3 | 2.2 | 13.6 |
| 8377-5-3-3-9 | 3 | 21.9 | 10.0 | 4.1 | 25.4 | 56.7 | 3.8 | 14.1 |
| 8377-5-3-3-37 | 3 | 21.7 | 8.6 | 4.1 | 41.3 | 42.9 | 3.1 | 12.7 |
| 8377-5-3-3-5 | 3 | 21.4 | 8.8 | 3.9 | 40.8 | 43.5 | 3.1 | 12.6 |
| 8377-5-3-3-10 | 3 | 21.4 | 10.8 | 3.9 | 24.3 | 56.6 | 4.4 | 14.7 |
| 8377-5-3-3-46 | 3 | 21.2 | 8.1 | 3.0 | 42.6 | 43.0 | 3.3 | 11.1 |
| 8377-5-3-3-39 | 3 | 21.1 | 10.4 | 4.0 | 33.0 | 48.5 | 4.1 | 14.5 |
| 8377-5-3-3-21 | 3 | 21.1 | 9.3 | 3.9 | 29.2 | 53.8 | 3.8 | 13.2 |
| 8377-5-3-3-36 | 3 | 21.0 | 9.2 | 3.7 | 45.2 | 39.8 | 2.2 | 12.9 |
| 8377-5-3-3-12 | 3 | 20.9 | 7.8 | 3.4 | 36.7 | 47.9 | 4.2 | 11.2 |
| 8377-5-3-3-19 | 3 | 20.9 | 9.0 | 3.8 | 49.7 | 34.3 | 3.2 | 12.8 |
| 8377-5-3-3-31 | 3 | 20.9 | 7.3 | 4.5 | 53.3 | 32.8 | 2.2 | 11.8 |
| 8377-5-3-3-26 | 3 | 20.9 | 7.6 | 3.6 | 34.9 | 50.8 | 3.1 | 11.2 |
| 8377-5-3-3-43 | 3 | 20.8 | 8.1 | 4.0 | 41.3 | 44.5 | 2.0 | 12.1 |
| 8377-5-3-3-41 | 3 | 20.6 | 10.0 | 3.6 | 49.4 | 34.0 | 2.9 | 13.6 |
| 8377-5-3-3-28 | 3 | 20.5 | 8.7 | 4.0 | 43.9 | 40.0 | 3.4 | 12.8 |
| 8377-5-3-3-22 | 3 | 20.4 | 8.5 | 3.1 | 39.9 | 44.9 | 3.7 | 11.5 |
| 8377-5-3-3-25 | 3 | 20.3 | 8.7 | 4.6 | 54.8 | 29.9 | 2.0 | 13.3 |
| 8377-5-3-3-47 | 3 | 20.2 | 8.9 | 3.6 | 44.8 | 39.8 | 2.8 | 12.5 |
| 8377-5-3-3-38 | 3 | 20.2 | 8.1 | 4.0 | 49.8 | 36.8 | 1.2 | 12.1 |
| 8377-5-3-3-7 | 3 | 20.1 | 9.5 | 4.3 | 40.3 | 42.5 | 3.4 | 13.8 |
| 8377-5-3-3-14 | 3 | 20.0 | 10.3 | 4.0 | 51.1 | 30.5 | 4.1 | 14.4 |
| 8377-5-3-3-1 | ? | 20.0 | 8.6 | 4.1 | 33.7 | 49.0 | 4.6 | 12.7 |
| 8377-5-3-3-3 | 3 | 19.8 | 7.6 | 3.1 | 46.5 | 39.0 | 3.9 | 10.7 |
| 8377-5-3-3-17 | 3 | 19.8 | 7.5 | 3.9 | 46.8 | 38.1 | 3.6 | 11.4 |
| 8377-5-3-3-29 | 3 | 19.6 | 8.1 | 4.1 | 36.8 | 47.8 | 3.2 | 12.2 |
| 8377-5-3-3-15 | 3 | 19.6 | 8.2 | 3.5 | 34.6 | 50.8 | 2.9 | 11.8 |
| 8377-5-3-3-18 | 3 | 19.6 | 8.2 | 2.3 | 53.8 | 33.7 | 2.0 | 10.5 |
| 8377-5-3-3-6 | 3 | 19.6 | 8.0 | 3.6 | 39.3 | 44.9 | 4.2 | 11.6 |
| 8377-5-3-3-20 | 3 | 19.5 | 8.8 | 3.8 | 44.8 | 39.3 | 3.4 | 12.6 |
| 8377-5-3-3-48 | 3 | 19.2 | 9.2 | 3.0 | 55.3 | 29.9 | 2.7 | 12.1 |
| 8377-5-3-3-2 | 3 | 19.1 | 7.6 | 3.8 | 50.7 | 35.6 | 2.3 | 11.4 |
| 8377-5-3-3-44 | 3 | 19.0 | 9.3 | 3.7 | 44.1 | 39.2 | 3.7 | 13.0 |
| 8377-5-3-3-34 | 3 | 18.9 | 11.1 | 3.9 | 39.3 | 43.1 | 2.5 | 15.1 |
| 8377-5-3-3-24 | 3 | 18.1 | 8.9 | 3.4 | 42.7 | 41.9 | 3.2 | 12.3 |
| Trans Avg. | | 20.4 | 8.8 | 3.8 | 41.6 | 42.7 | 3.1 | 12.6 |
| 8377-5-3-3-45 | 0 | 20.1 | 10.6 | 3.4 | 33.8 | 43.0 | 9.3 | 13.9 |
| 8377-5-3-3-4 | 0 | 19.3 | 9.5 | 3.0 | 22.3 | 52.3 | 12.9 | 12.6 |
| 8377-5-3-3-27 | 0 | 18.6 | 10.9 | 3.4 | 24.5 | 48.1 | 13.1 | 14.3 |
| 8377-5-3-3-23 | 0 | 18.4 | 10.7 | 3.4 | 32.5 | 42.9 | 10.6 | 14.1 |
| 8377-5-3-3-40 | 0 | 18.4 | 9.5 | 2.7 | 45.8 | 33.8 | 8.1 | 12.3 |
| 8377-5-3-3-8 | 0 | 18.4 | 10.3 | 3.0 | 23.1 | 49.3 | 14.3 | 13.3 |
| 8377-5-3-3-32 | 0 | 18.1 | 10.7 | 3.0 | 16.4 | 55.8 | 14.1 | 13.7 |
| 8377-5-3-3-11 | 0 | 18.1 | 10.1 | 2.4 | 28.6 | 45.3 | 13.7 | 12.4 |
| 8377-5-3-3-42 | 0 | 18.1 | 13.5 | 3.9 | 38.6 | 35.4 | 8.5 | 17.5 |
| 8377-5-3-3-35 | 0 | 17.8 | 11.4 | 3.9 | 26.7 | 47.2 | 10.9 | 15.2 |
| 8377-5-3-3-13 | 0 | 17.0 | 10.7 | 2.6 | 26.8 | 48.6 | 11.2 | 13.4 |
| Null Avg. | | 18.4 | 10.7 | 3.1 | 29.0 | 45.6 | 11.5 | 13.9 |
| 8377-5-3-4-23 | 3 | 23.6 | 10.5 | 3.8 | 30.1 | 52.5 | 3.1 | 14.4 |
| 8377-5-3-4-27 | 3 | 22.5 | 9.0 | 4.2 | 36.2 | 48.3 | 2.4 | 13.2 |
| 8377-5-3-4-11 | 3 | 22.5 | 8.9 | 4.0 | 37.8 | 47.5 | 1.7 | 13.0 |
| 8377-5-3-4-30 | 3 | 22.4 | 7.4 | 4.6 | 50.2 | 36.0 | 1.7 | 12.1 |
| 8377-5-3-4-18 | 3 | 22.1 | 9.0 | 5.2 | 32.9 | 50.5 | 2.3 | 14.2 |
| 8377-5-3-4-9 | 3 | 21.9 | 8.8 | 3.1 | 42.1 | 42.9 | 3.1 | 11.9 |
| 8377-5-3-4-7 | 3 | 21.7 | 8.9 | 3.2 | 32.3 | 52.5 | 3.1 | 12.1 |
| 8377-5-3-4-4 | 3 | 21.7 | 9.2 | 3.6 | 44.8 | 40.5 | 1.9 | 12.8 |
| 8377-5-3-4-19 | 3 | 21.5 | 9.5 | 4.8 | 36.6 | 46.1 | 2.9 | 14.3 |
| 8377-5-3-4-16 | 3 | 21.4 | 7.3 | 4.2 | 49.3 | 36.7 | 2.5 | 11.5 |
| 8377-5-3-4-25 | 3 | 21.0 | 9.2 | 3.7 | 38.5 | 46.1 | 2.6 | 12.9 |
| 8377-5-3-4-6 | 3 | 20.8 | 8.6 | 3.9 | 34.9 | 49.4 | 3.2 | 12.5 |
| 8377-5-3-4-26 | 3 | 20.8 | 9.9 | 4.3 | 28.7 | 53.4 | 3.7 | 14.2 |
| 8377-5-3-4-20 | 3 | 20.7 | 9.0 | 6.1 | 29.9 | 51.6 | 3.3 | 15.1 |
| 8377-5-3-4-22 | 3 | 20.4 | 8.8 | 4.2 | 44.9 | 40.3 | 1.7 | 13.0 |
| 8377-5-3-4-13 | 3 | 20.3 | 9.2 | 3.8 | 44.7 | 40.0 | 2.3 | 13.0 |
| 8377-5-3-4-2 | 3 | 20.2 | 8.2 | 3.4 | 42.2 | 43.3 | 2.8 | 11.6 |
| 8377-5-3-4-29 | 3 | 20.1 | 8.4 | 4.7 | 51.7 | 33.4 | 1.8 | 13.1 |
| 8377-5-3-4-33 | 3 | 19.7 | 7.9 | 4.5 | 33.2 | 52.3 | 2.1 | 12.4 |
| 8377-5-3-4-10 | 3 | 19.7 | 11.3 | 4.4 | 34.3 | 47.7 | 2.4 | 15.7 |
| 8377-5-3-4-15 | 3 | 19.4 | 8.1 | 3.9 | 50.6 | 35.6 | 1.8 | 11.9 |
| 8377-5-3-4-5 | 3 | 19.2 | 8.4 | 3.0 | 54.7 | 31.5 | 2.3 | 11.4 |
| 8377-5-3-4-12 | 3 | 19.0 | 7.7 | 4.6 | 45.8 | 40.3 | 1.6 | 12.3 |
| 8377-5-3-4-8 | 3 | 18.9 | 11.1 | 4.0 | 34.4 | 47.7 | 2.8 | 15.1 |
| 8377-5-3-4-28 | 3 | 18.9 | 9.1 | 4.4 | 49.7 | 34.9 | 1.9 | 13.4 |
| 8377-5-3-4-3 | 3 | 18.5 | 8.6 | 3.4 | 58.3 | 28.4 | 1.2 | 12.1 |
| 8377-5-3-4-17 | 3 | 17.4 | 9.1 | 4.0 | 24.2 | 58.2 | 4.5 | 13.1 |
| 8377-5-3-4-24 | 3 | 16.1 | 7.3 | 4.3 | 49.1 | 38.1 | 1.2 | 11.6 |
| Trans Avg. | | 20.4 | 8.9 | 4.1 | 40.8 | 43.8 | 2.4 | 13.0 |
| 8377-5-3-4-21 | 0 | 19.5 | 9.9 | 3.2 | 38.4 | 40.2 | 8.4 | 13.0 |
| 8377-5-3-4-1 | 0 | 19.1 | 11.2 | 3.6 | 20.7 | 51.4 | 13.1 | 14.8 |
| 8377-5-3-4-14 | 0 | 18.9 | 9.0 | 3.2 | 43.1 | 35.6 | 9.1 | 12.2 |
| 8377-5-3-4-32 | 0 | 15.9 | 13.0 | 4.7 | 39.1 | 33.4 | 9.8 | 17.7 |
| 8377-5-3-4-31 | 0 | 15.3 | 10.7 | 3.7 | 24.2 | 50.6 | 10.8 | 14.4 |
| Null Avg. | | 17.7 | 10.7 | 3.7 | 33.1 | 42.2 | 10.3 | 14.4 |
| 8377-1-2-1-26 | 3 | 25.0 | 8.3 | 6.4 | 30.6 | 52.7 | 2.0 | 14.7 |
| 8377-1-2-1-11 | 3 | 24.8 | 8.0 | 5.5 | 34.2 | 49.8 | 2.4 | 13.5 |
| 8377-1-2-1-32 | 3 | 24.8 | 8.4 | 6.9 | 30.4 | 52.3 | 2.0 | 15.2 |
| 8377-1-2-1-18 | 3 | 24.7 | 8.4 | 4.9 | 31.2 | 52.6 | 2.9 | 13.3 |
| 8377-1-2-1-33 | 3 | 24.5 | 8.5 | 5.8 | 28.9 | 53.8 | 3.0 | 14.3 |
| 8377-1-2-1-30 | 3 | 24.3 | 7.8 | 7.1 | 30.4 | 52.7 | 2.0 | 14.9 |
| 8377-1-2-1-8 | 3 | 24.1 | 9.0 | 4.9 | 28.7 | 54.3 | 3.1 | 13.9 |
| 8377-1-2-1-35 | 3 | 24.1 | 9.6 | 5.1 | 26.7 | 55.1 | 3.6 | 14.7 |
| 8377-1-2-1-4 | 3 | 23.6 | 8.3 | 7.3 | 30.6 | 51.6 | 2.2 | 15.6 |
| 8377-1-2-1-36 | 3 | 23.5 | 7.5 | 6.4 | 32.2 | 52.3 | 1.6 | 13.9 |
| 8377-1-2-1-7 | 3 | 23.3 | 8.0 | 6.1 | 29.9 | 54.1 | 1.9 | 14.1 |
| 8377-1-2-1-21 | 3 | 22.9 | 8.9 | 6.0 | 24.4 | 57.1 | 3.6 | 14.9 |
| 8377-1-2-1-5 | 3 | 22.8 | 9.9 | 6.1 | 29.8 | 52.1 | 2.1 | 16.0 |
| 8377-1-2-1-25 | 3 | 22.7 | 9.0 | 5.1 | 29.8 | 52.9 | 3.2 | 14.1 |
| 8377-1-2-1-10 | 3 | 22.6 | 9.2 | 6.2 | 26.0 | 55.2 | 3.3 | 15.5 |
| 8377-1-2-1-19 | 3 | 22.4 | 9.6 | 4.8 | 25.6 | 55.8 | 4.3 | 14.4 |
| 8377-1-2-1-28 | 3 | 22.2 | 9.1 | 4.7 | 31.3 | 51.3 | 3.6 | 13.8 |
| 8377-1-2-1-29 | 3 | 21.9 | 8.2 | 4.8 | 32.9 | 51.8 | 2.3 | 12.9 |
| 8377-1-2-1-22 | 3 | 21.9 | 10.1 | 5.5 | 23.5 | 57.7 | 3.1 | 15.6 |
| 8377-1-2-1-1 | 3 | 21.7 | 9.8 | 5.3 | 25.6 | 55.8 | 3.4 | 15.1 |
| 8377-1-2-1-9 | 3 | 21.6 | 9.8 | 5.2 | 24.6 | 55.9 | 4.6 | 15.0 |
| 8377-1-2-1-31 | 3 | 21.4 | 8.8 | 4.4 | 32.4 | 51.8 | 2.6 | 13.2 |
| 8377-1-2-1-6 | 3 | 20.9 | 9.2 | 5.8 | 23.5 | 57.2 | 4.2 | 15.0 |
| 8377-1-2-1-15 | 3 | 20.7 | 9.5 | 4.4 | 31.8 | 51.8 | 2.6 | 13.9 |
| 8377-1-2-1-14 | 3 | 20.6 | 9.0 | 5.0 | 28.7 | 54.0 | 3.3 | 14.0 |
| 8377-1-2-1-16 | 3 | 20.3 | 9.1 | 4.9 | 32.1 | 50.9 | 3.1 | 14.0 |
| 8377-1-2-1-17 | 3 | 20.2 | 9.1 | 5.6 | 28.6 | 53.4 | 3.3 | 14.7 |
| 8377-1-2-1-20 | 3 | 18.8 | 9.2 | 6.1 | 22.9 | 57.2 | 4.4 | 15.4 |
| 8377-1-2-1-23 | 3 | 17.8 | 9.8 | 6.1 | 23.8 | 56.4 | 3.9 | 15.9 |
| Trans Avg. | | 22.4 | 8.9 | 5.6 | 28.7 | 53.8 | 3.0 | 14.5 |
| 8377-1-2-1-24 | 0 | 19.8 | 11.0 | 4.4 | 16.7 | 53.9 | 14.1 | 15.4 |
| 8377-1-2-1-34 | 0 | 19.8 | 11.1 | 5.0 | 17.3 | 53.6 | 13.0 | 16.1 |
| 8377-1-2-1-3 | 0 | 19.1 | 10.0 | 3.9 | 21.9 | 52.2 | 12.0 | 13.9 |
| 8377-1-2-1-2 | 0 | 18.4 | 11.1 | 4.3 | 16.9 | 53.9 | 13.9 | 15.3 |
| 8377-1-2-1-13 | 0 | 18.3 | 11.3 | 3.9 | 14.5 | 54.8 | 15.4 | 15.2 |
| 8377-1-2-1-12 | 0 | 17.3 | 10.9 | 3.8 | 14.1 | 51.9 | 19.2 | 14.7 |
| 8377-1-2-1-27 | 0 | 17.1 | 12.3 | 4.5 | 15.7 | 52.1 | 15.4 | 16.8 |
| Null Avg. | | 18.5 | 11.1 | 4.3 | 16.7 | 53.2 | 14.7 | 15.4 |

¹T1 seed description, e.g. AFS 8407-2-2-1-74: AFS8407-2-2 (=event), AFS8407-2-2-1 (=plant 1 of event AFS8407-2-2-1, AFS8407-2-2-1-74 (=T1 seed 74 from plant 1 of event AFS8407-2-2-1).

From Table 3, it can be seen that transgenic seed have higher oil and oleic acid content than null seed and also have lower alpha-linolenic content compared to null seed. Also, transgenic seed show decreased raffinosaccharides and increased sucrose by TLC compared to null seed.

Additionally, it can be seen that all events segregate for phenotype (TLC result and lower alpha-linolenic acid) in a 3:1 Mendelian fashion except for event AFS 8377-1-4. Southern blot data indicated that this event has an extra copy of donor DNA which in this case is giving rise to a phenotype in all transgenic seed and is thus, the extra copy is functional. This extra copy can be segregated away in later generations in event AFS 8377-1-4. Southern blot analysis of T0 tissue of AFS 3877-4-4 and AFS 8377-5-3 tissue had also indicated and extra copy of DNA but in those cases, the extra copy did not give rise to a phenotype and could also be segregated way.

A summary of the average values for oil content and fatty acid profile for T1 seed from each event, along with the difference in oil content for each event is shown in Table 4. In Table 4, the percent change of transgenic compared to null is indicated as % Change.

TABLE 4
Average oil contents and fatty acid profiles for T1 seed from RMCE events
from Soil19.

| T0 | | | | | | | | |
|---|---|---|---|---|---|---|---|---|
| Plant | T1 Seed | % Oil | 16:0% | 18:0% | 18:1% | 18:2% | 18:3% | Sats |
| AFS | Null Avg. | 18.5 | 11.1 | 4.3 | 16.7 | 53.2 | 14.7 | 15.4 |
| 8377.1.2.1 | | | | | | | | |
| AFS | Trans Avg. | 22.4 | 8.9 | 5.6 | 28.7 | 53.8 | 3.0 | 14.5 |
| 8377.1.2.1 | | | | | | | | |
| | % Change | 21 | −19 | 32 | 71 | 1 | −79 | −5 |
| AFS | Null Avg. | 21.5 | 10.4 | 3.8 | 16.6 | 55.3 | 13.9 | 14.1 |
| 8377.1.4.2 | | | | | | | | |
| AFS | Trans Avg. | 23.1 | 9.2 | 4.7 | 28.0 | 55.6 | 2.5 | 13.9 |
| 8377.1.4.2 | | | | | | | | |
| | % Change | 7 | −11 | 24 | 68 | 1 | −82 | −2 |
| AFS | Null Avg. | 18.6 | 9.8 | 3.5 | 19.8 | 52.6 | 14.4 | 13.3 |
| 8377.1.4.3 | | | | | | | | |
| AFS | Trans Avg. | 20.9 | 8.2 | 5.1 | 36.1 | 47.7 | 2.9 | 13.3 |
| 8377.1.4.3 | | | | | | | | |
| | % Change | 12 | −16 | 46 | 83 | −9 | −80 | 0 |
| AFS | Null Avg. | 18.4 | 10.7 | 3.1 | 29.0 | 45.6 | 11.5 | 13.9 |
| 8377.5.3.3 | | | | | | | | |
| AFS | Trans Avg. | 20.4 | 8.8 | 3.8 | 41.6 | 42.7 | 3.1 | 12.6 |
| 8377.5.3.3 | | | | | | | | |
| | % Change | 11 | −18 | 19 | 43 | −6 | −73 | −9 |
| AFS | Null Avg. | 17.7 | 10.7 | 3.7 | 33.1 | 42.2 | 10.3 | 14.4 |
| 8377.5.3.4 | | | | | | | | |
| AFS | Trans Avg. | 20.4 | 8.9 | 4.1 | 40.8 | 43.8 | 2.4 | 13.0 |
| 8377.5.3.4 | | | | | | | | |
| | % Change | 15 | −17 | 12 | 23 | 4 | −76 | −10 |
| AFS | Null Avg. | 22.5 | 11.4 | 3.7 | 17.2 | 54.6 | 13.1 | 15.1 |
| 8407.2.2.1 | | | | | | | | |
| AFS | Trans Avg. | 25.8 | 9.2 | 4.7 | 27.8 | 55.4 | 2.9 | 13.9 |
| 8407.2.2.1 | | | | | | | | |
| | % Change | 14 | −19 | 27 | 62 | 1 | −78 | −8 |
| AFS | Null Avg. | 20.8 | 10.7 | 3.6 | 22.1 | 51.9 | 11.8 | 14.3 |
| 8407.2.2.2 | | | | | | | | |
| AFS | Trans Avg. | 23.9 | 8.6 | 4.6 | 34.2 | 50.2 | 2.3 | 13.3 |
| 8407.2.2.2 | | | | | | | | |
| | % Change | 15 | −19 | 29 | 55 | −3 | −80 | −7 |
| AFS | Null Avg. | 19.7 | 10.8 | 3.6 | 17.9 | 54.3 | 13.5 | 14.4 |
| 8407.2.2.3 | | | | | | | | |
| AFS | Trans Avg. | 23.0 | 9.0 | 4.7 | 29.1 | 54.3 | 2.9 | 13.7 |
| 8407.2.2.3 | | | | | | | | |
| | % Change | 17 | −16 | 30 | 63 | 0 | −79 | −5 |
| AFS | Null Avg. | 22.8 | 11.0 | 4.2 | 18.1 | 54.0 | 12.6 | 15.2 |
| 8407.2.2.4 | | | | | | | | |
| AFS | Trans Avg. | 25.4 | 8.6 | 5.0 | 29.3 | 54.0 | 3.1 | 13.6 |
| 8407.2.2.4 | | | | | | | | |
| | % Change | 12 | −22 | 19 | 62 | 0 | −76 | −11 |

In Table 4, average oil content percent increases comparing all transgenic seed to null segregant T1 seed range from 7 to 21% over null. Palmitic acid average percent decrease ranges from 11 to 19%. Stearic acid average percent increase ranges from 12 to 46%. Total Sats decrease ranges from 0 to 11%. Oleic acid average percent increase ranges from 23 to 83%. Linoleic acid increases or decreases slightly (−9% to 1% change). Alpha-linolenic acid average percent decrease ranges from 73 to 82%.

### Example 5

### Compositional Analysis of Soybean Seed

The present example describes measurements of seed compositional parameters such as protein content and content of soluble carbohydrates of soybean seed derived from transgenic events. To this end the concentrations of protein, soluble carbohydrates and starch were measured as follows.

### Non-Structural Carbohydrate and Protein Analysis.

Dry soybean seed were ground to a fine powder in a GenoGrinder and subsamples were weighed (to an accuracy of 0.1 mg) into 13×100 mm glass tubes; the tubes had Teflon® lined screw-cap closures. Three replicates were prepared for each sample tested. Tissue dry weights were calculated by weighing sub-samples before and after drying in a forced air oven for 18 h at 105 C.

Lipid extraction was performed by adding 2 ml aliquots of heptane to each tube. The tubes were vortex mixed and placed into an ultrasonic bath (VWR Scientific Model 750D) filled with water heated to 60 C. The samples were sonicated at full-power (˜360 W) for 15 min and were then centrifuged (5 min×1700 g). The supernatants were transferred to clean 13×100 mm glass tubes and the pellets were extracted 2 more times with heptane (2 ml, second extraction, 1 ml third extraction) with the supernatants from each extraction being pooled. After lipid extraction 1 ml acetone was added to the pellets and after vortex mixing, to fully disperse the material, they were taken to dryness in a Speedvac.

### Non-Structural Carbohydrate Extraction and Analysis.

Two ml of 80% ethanol was added to the dried pellets from above. The samples were thoroughly vortex mixed until the plant material was fully dispersed in the solvent prior to sonication at 60 C for 15 min. After centrifugation, 5 min×1700 g, the supernatants were decanted into clean 13×100 mm glass tubes. Two more extractions with 80% ethanol were performed and the supernatants from each were pooled. The extracted pellets were suspended in acetone and dried (as above). An internal standard β-phenyl glucopyranoside (100 ul of a 0.5000+/−0.0010 g/100 ml stock) was added to each extract prior to drying in a Speedvac. The extracts were maintained in a desiccator until further analysis.

The acetone dried powders from above were suspended in 0.9 ml MOPS (3-N[Morpholino]propane-sulfonic acid; 50 mM, 5 mM CaCl₂, pH 7.0) buffer containing 1000 of heat stable α-amylase (from Bacillus licheniformis; Sigma A-4551). Samples were placed in a heat block (90 C) for 75 min and were vortex mixed every 15 min. Samples were then allowed to cool to room temperature and 0.6 ml acetate buffer (285 mM, pH 4.5) containing 5 U amyloglucosidase (Roche 110 202 367 001) was added to each. Samples were incubated for 15-18 h at 55 C in a water bath fitted with a reciprocating shaker; standards of soluble potato starch (Sigma S-2630) were included to ensure that starch digestion went to completion.

Post-digestion the released carbohydrates were extracted prior to analysis. Absolute ethanol (6 ml) was added to each tube and after vortex mixing the samples were sonicated for 15 min at 60 C. Samples were centrifuged (5 min×1700 g) and the supernatants were decanted into clean 13×100 mm glass tubes. The pellets were extracted 2 more times with 3 ml of 80% ethanol and the resulting supernatants were pooled. Internal standard (100 ul [symbol] L-phenyl glucopyranoside, as above) was added to each sample prior to drying in a Speedvac.

### Sample Preparation and Analysis

The dried samples from the soluble and starch extractions described above were solubilized in anhydrous pyridine (Sigma-Aldrich P57506) containing 30 mg/ml of hydroxylamine HCl (Sigma-Aldrich 159417). Samples were placed on an orbital shaker (300 rpm) overnight and were then heated for 1 hr (75 C) with vigorous vortex mixing applied every 15 min. After cooling to room temperature 1 ml hexamethyldisilazane (Sigma-Aldrich H-4875) and 100 ul trifluoroacetic acid (Sigma-Aldrich T-6508) were added. The samples were vortex mixed and the precipitates were allowed to settle prior to transferring the supernatants to GC sample vials.

Samples were analyzed on an Agilent 6890 gas chromatograph fitted with a DB-17MS capillary column (15 m×0.32 mm×0.25 um film). Inlet and detector temperatures were both 275 C. After injection (2 ul, 20:1 split) the initial column temperature (150 C) was increased to 180 C at a rate 3 C/min and then at 25 C/min to a final temperature of 320 C. The final temperature was maintained for 10 min. The carrier gas was H₂ at a linear velocity of 51 cm/sec. Detection was by flame ionization. Data analysis was performed using Agilent ChemStation software. Each sugar was quantified relative to the internal standard and detector responses were applied for each individual carbohydrate (calculated from standards run with each set of samples). Final carbohydrate concentrations were expressed on a tissue dry weight basis.

### Protein Analysis

Protein contents were estimated by combustion analysis on a Thermo Finnigan Flash 1112EA combustion analyzer. Samples, 4-8 mg, weighed to an accuracy of 0.001 mg on a Mettler-Toledo MX5 micro balance were used for analysis. Protein contents were calculated by multiplying % N, determined by the analyzer, by 6.25. Final protein contents were expressed on a percent tissue dry weight basis.

### Example 6

### Compositional Analysis of T1 Seed from Soil19 Event/Plant AFS 8407.2.2.1

Eight transgenic seed from Soil19 Event/Plant AFS 8407.2.2.1 (8407-2-2-1-74, 8407-2-2-1-53, 8407-2-2-1-65, 8407-2-2-1-80, 8407-2-2-1-60, 8407-2-2-1-51, 8407-2-2-1-70, 8407-2-2-1-63 from Table 3) and the eight null seed from Soil19 Event/Plant AFS 8407-2-2-1 (seed 8407-2-2-1-61, 8407-2-2-1-69, 8407-2-2-1-66, 8407-2-2-1-50, 8407-2-2-1-62, 8407-2-2-1-64, 8407-2-2-1-58, 8407-2-2-1-68 from Table 3) were combined together, respectively, and ground to a powder using the genogrinder as described herein.

Non-structural soluble carbohydrate and protein from the chosen Soil19 T1 seed transgenic and null powders were quantified using the methods described in Example 5 and oil content of powders was determined by NMR as described herein and results are presented in Table 5a.

In Table 5a, individual soluble carbohydrates (pinitol, sorbitol, fructose, glucose, sucrose, galactinol, raffinose, stachyose) as well as protein and oil are reported as a percent of ground soy powder. Also presented are the total rafinosaccharides (Total Rafs; sum of raffinose and stachyose) and total soluble carbohydrates (Total Carbs; sum of individual carbohydrates). Percent increase or decrease in any particular soluble carbohydrate or protein or oil as is also shown in Table 5a where the percent is calculated in the following way; [(transgenic value-null value)/null value×100%].

TABLE 5a
Soluble carbohydrate, protein and oil content of bulk powders from null
and transgenic Soil19 event AFS 8407.2.2.1 soybean.
Soluble Carboydrates

| Sample | Pinitol | Sorbitol | Fructose | Glucose | Sucrose | Galactinol | Raffinose | Stachyose | Total Rafs | Total Carb | Protein | Oil |
|---|---|---|---|---|---|---|---|---|---|---|---|---|
| Soil 19 | 0.25 | 0 | 0.02 | 0.05 | 4.88 | 0.22 | 1.04 | 5.74 | 6.79 | 12.2 | 37.5 | 22.8 |
| Null | | | | | | | | | | | | |
| Soil 19 | 0.49 | 0 | 0.00 | 0.02 | 6.56 | 0.31 | 0.75 | 0.44 | 1.19 | 8.5 | 37.7 | 27.5 |
| Trans | | | | | | | | | | | | |
| % Change | 96 | | −100 | −57 | 35 | 41 | −28 | −92 | −83 | −30 | 1 | 20 |

Table 5a shows a percent increase in oil in transgenic Soil19 seed of 20%, a percent increase in protein of 1%, a percent increase in sucrose of 35%, a percent decrease in raffinosaccharides of 83% and a percent decrease in total carbohydrates of 30%, when compared to null segregant Soil19 soybean seed.

A soybean meal can be generated by one skilled in the art by extracting the oil component away from the ground seed powder. Given the oil, protein and total soluble carbohydrate compositions shown in Table 5a, the resulting protein and soluble carbohydrate compositions can be calculated for a soybean meal and these are shown in Table 5b.

In Table 5b, individual soluble carbohydrates (pinitol, sorbitol, fructose, glucose, sucrose, galactinol, raffinose, stachyose) and protein are reported as a percent of soybean meal. Also presented are the total rafinosaccharides (Total Rafs; sum of raffinose and stachyose) and total soluble carbohydrates (Total Carbs; sum of individual carbohydrates). Soybean meal composition for each component was calculated by dividing the percent of an individual meal component in soybean powder by (100 percent minus the percent oil in soybean powder). For example, in the case of the Soil19 null shown in Table 5b, the percent protein in the soybean meal becomes [% protein/(100-% oil)=37.5/(100-22.8)] which results in 48.6% protein in soybean meal from that seed. A similar calculation was performed for each component from Table 5a and is shown in Table 5b. Percent increase or decrease for each component in the soybean meal is also shown in Table 5b where the percent is calculated in the following way; [(transgenic value-null value)/null value×100%].

TABLE 5b
Soluble carbohydrate and protein of soybean meal generated from null
and transgenic Soil19 event AFS 8407.2.2.1 soybean.

| Sample | Pinitol | Sorbitol | Fructose | Glucose | Sucrose | Galactinol | Raffinose | Stachyose | Total Rafs | Total Carbs | Protein |
|---|---|---|---|---|---|---|---|---|---|---|---|
| Soil19 | 0.3 | 0.0 | 0.0 | 0.1 | 6.3 | 0.3 | 1.3 | 7.4 | 8.8 | 15.8 | 48.6 |
| Null | | | | | | | | | | | |
| Soil19 | 0.7 | 0.0 | 0.0 | 0.0 | 9.0 | 0.4 | 1.0 | 0.6 | 1.6 | 11.8 | 52.0 |
| Trans | | | | | | | | | | | |
| % | 109 | | −100 | −57 | 43 | 50 | −23 | −92 | −81 | −25 | 7 |
| Change | | | | | | | | | | | |

Table 5b shows an increase in protein in transgenic Soil19 soybean meal of 7%, an increase in sucrose of 43%, a decrease in raffinosaccharides of 81% and a decrease in total carbohydrates of 25%, when compared to null segregant Soil19 soybean meal.

Compositional Analysis of T1 Seed from Soil19 Event/Plant AFS 8377.1.2.1

The thirty six individual T1 seed from Soil19 Event/Plant AFS 8377.1.2.1 (Tables 3&4) were ground to a powder using the genogrinder as described herein. Non-structural soluble carbohydrate and protein from each transgenic and null powders (as determined with fatty acid profile and TLC result in Table 3 were quantified using the methods described in Example 2 and oil content of powders was determined by NMR as described herein and results are presented in Table 6a.

In Table 6a, individual soluble carbohydrates (pinitol, sorbitol, fructose, glucose, sucrose, galactinol, raffinose, stachyose) as well as protein and oil are for individual seed are reported as a percent of ground soy powder. Also presented are the total rafinosaccharides (Total Rafs; sum of raffinose and stachyose) and total soluble carbohydrates (Total Carbs; sum of individual carbohydrates).

In Table 6a, the average value for all transgenic seed or null seed is shown (Avg.). Additionally, the percent increase or decrease (percent change) for the average of any particular soluble carbohydrate, protein or oil as is also shown in Table 6a where the percent is calculated in the following way; [(transgenic value-null value)/null value×100%].

TABLE 6a
Fatty acid profile and soluble carbohydrate, protein and oil content of
individual seed from null and transgenic Soil19 event AFS 8377.1.2.1 soybean.

| Seed # | Pinitol | Sorbitol | Fructose | Glucose | Sucrose | Galactinol | Raffinose | Stachyose | Total Rafs | Total Carbs | Protein | Oil |
|---|---|---|---|---|---|---|---|---|---|---|---|---|
| 36 | 0.70 | 0 | 0.07 | 0.06 | 9.03 | 0.02 | 0.58 | 0.25 | 0.82 | 10.71 | 43.1 | 23.5 |
| 7 | 0.64 | 0 | 0.11 | 0.12 | 7.14 | 0.02 | 0.28 | 0.12 | 0.41 | 8.43 | 45.6 | 23.3 |
| 11 | 0.46 | 0 | 0.09 | 0.08 | 8.74 | 0.02 | 0.50 | 0.24 | 0.74 | 10.14 | 39.7 | 24.8 |
| 30 | 0.60 | 0 | 0.11 | 0.14 | 6.70 | 0.02 | 0.36 | 0.22 | 0.57 | 8.14 | 45.0 | 24.3 |
| 26 | 0.62 | 0 | 0.08 | 0.07 | 6.99 | 0.02 | 0.42 | 0.26 | 0.68 | 8.45 | 43.7 | 25.0 |
| 4 | 0.70 | 0 | 0.08 | 0.07 | 6.69 | 0.02 | 0.34 | 0.22 | 0.55 | 8.11 | 44.9 | 23.6 |
| 5 | 0.48 | 0 | 0.12 | 0.16 | 7.59 | 0.01 | 0.36 | 0.18 | 0.55 | 8.90 | 46.3 | 22.8 |
| 31 | 0.55 | 0 | 0.11 | 0.14 | 8.15 | 0.02 | 0.42 | 0.22 | 0.64 | 9.61 | 47.1 | 21.4 |
| 29 | 0.68 | 0 | 0.12 | 0.12 | 7.42 | 0.02 | 0.46 | 0.30 | 0.76 | 9.13 | 43.9 | 21.9 |
| 15 | 0.44 | 0 | 0.08 | 0.10 | 8.01 | 0.02 | 0.41 | 0.26 | 0.67 | 9.33 | 47.4 | 20.7 |
| 8 | 0.53 | 0 | 0.10 | 0.15 | 7.31 | 0.02 | 0.51 | 0.39 | 0.90 | 9.01 | 43.8 | 24.1 |
| 8 | 0.74 | 0 | 0.11 | 0.09 | 6.96 | 0.02 | 0.61 | 0.58 | 1.19 | 9.12 | 42.2 | 24.7 |
| 33 | 0.78 | 0 | 0.07 | 0.05 | 8.00 | 0.03 | 0.82 | 0.63 | 1.45 | 10.38 | 41.6 | 24.5 |
| 25 | 0.52 | 0 | 0.10 | 0.07 | 9.07 | 0.03 | 0.82 | 0.74 | 1.57 | 11.36 | 43.7 | 22.7 |
| 19 | 0.80 | 0 | 0.11 | 0.08 | 7.50 | 0.02 | 0.62 | 0.57 | 1.19 | 9.70 | 43.0 | 22.4 |
| 9 | 0.49 | 0 | 0.04 | 0.06 | 8.61 | 0.02 | 0.52 | 0.37 | 0.89 | 10.10 | 48.4 | 21.6 |
| 16 | 0.71 | 0 | 0.10 | 0.08 | 8.51 | 0.24 | 0.49 | 0.23 | 0.72 | 10.35 | 47.6 | 20.3 |
| 22 | 0.63 | 0 | 0.03 | 0.04 | 6.94 | 0.02 | 0.51 | 0.49 | 0.99 | 8.66 | 47.5 | 21.9 |
| 35 | 0.51 | 0 | 0.06 | 0.05 | 8.24 | 0.02 | 0.66 | 0.47 | 1.13 | 10.01 | 41.7 | 24.1 |
| 14 | 0.51 | 0 | 0.08 | 0.05 | 7.00 | 0.25 | 0.44 | 0.42 | 0.86 | 8.75 | 49.1 | 20.6 |
| 17 | 0.99 | 0 | 0.11 | 0.07 | 6.85 | 0.02 | 0.49 | 0.40 | 0.88 | 8.92 | 50.0 | 20.2 |
| 21 | 0.46 | 0 | 0.03 | 0.04 | 6.48 | 0.02 | 0.55 | 0.60 | 1.15 | 8.19 | 47.0 | 22.9 |
| 1 | 0.26 | 0 | 0.04 | 0.05 | 3.69 | 0.01 | 0.24 | 0.13 | 0.37 | 4.43 | 44.8 | 21.7 |
| 6 | 0.73 | 0 | 0.10 | 0.15 | 7.17 | 0.02 | 0.55 | 0.44 | 0.98 | 9.15 | 48.0 | 20.9 |
| 28 | 0.43 | 0 | 0.08 | 0.07 | 7.46 | 0.02 | 0.67 | 0.65 | 1.32 | 9.37 | 44.2 | 22.2 |
| 10 | 0.44 | 0 | 0.04 | 0.07 | 7.38 | 0.02 | 0.60 | 0.55 | 1.14 | 9.09 | 46.7 | 22.6 |
| 32 | 0.51 | 0 | 0.10 | 0.12 | 6.98 | 0.02 | 0.45 | 0.27 | 0.72 | 8.45 | 45.6 | 24.8 |
| 23 | 0.85 | 0 | 0.03 | 0.04 | 6.11 | 0.01 | 0.58 | 0.45 | 1.03 | 8.08 | 53.1 | 17.8 |
| 20 | 0.82 | 0 | 0.09 | 0.09 | 6.53 | 0.02 | 0.51 | 0.40 | 0.91 | 8.45 | 48.1 | 18.8 |
| Avg. | 0.61 | 0 | 0.08 | 0.08 | 7.35 | 0.04 | 0.51 | 0.38 | 0.89 | 9.05 | 45.6 | 22.4 |
| Trans | | | | | | | | | | | | |
| 24 | 0.29 | 0 | 0.04 | 0.03 | 5.51 | 0.07 | 0.61 | 5.71 | 6.32 | 12.26 | 42.6 | 19.8 |
| 34 | 0.33 | 0 | 0.04 | 0.04 | 5.13 | 0.05 | 0.68 | 5.49 | 6.17 | 11.76 | 42.5 | 19.8 |
| 3 | 0.26 | 0 | 0.09 | 0.06 | 4.90 | 0.06 | 1.07 | 6.51 | 7.58 | 12.95 | 44.1 | 19.1 |
| 2 | 0.32 | 0 | 0.07 | 0.04 | 5.72 | 0.04 | 0.85 | 4.32 | 5.17 | 11.36 | 46.2 | 18.4 |
| 13 | 0.22 | 0 | 0.10 | 0.09 | 6.92 | 0.04 | 0.71 | 4.96 | 5.67 | 13.03 | 41.8 | 18.3 |
| 12 | 0.21 | 0 | 0.04 | 0.04 | 5.92 | 0.03 | 0.80 | 4.84 | 5.64 | 11.89 | 44.4 | 17.3 |
| 27 | 0.43 | 0 | 0.05 | 0.04 | 4.22 | 0.05 | 1.04 | 4.67 | 5.70 | 10.50 | 49.0 | 17.1 |
| Avg. | 0.29 | 0 | 0.06 | 0.05 | 5.47 | 0.05 | 0.82 | 5.21 | 6.04 | 11.96 | 44.4 | 18.5 |
| Null | | | | | | | | | | | | |
| % | 105.7 | | 34.3 | 71.7 | 34.3 | −28.4 | −38.1 | −92.7 | −85.3 | −24.3 | 2.8 | 20.9 |
| Change | | | | | | | | | | | | |

Table 6a shows an increase in oil in transgenic Soil19 seed of 21%, an increase in protein of 3%, an increase in sucrose of 34%, a decrease in raffinosaccharides of 85%, and a decrease in total carbohydrates of 24% when compared to null segregants Soil19 soybean seed.

A soybean meal can be generated by one skilled in the art and the resulting protein and soluble carbohydrate compositions can be calculated for the resulting soybean meal using the composition obtained for the seed as described above. Given the average oil, protein and total soluble carbohydrate compositions shown in Table 6a, the resulting average protein and soluble carbohydrate compositions can be calculated for a soybean meal, as described above, and these are shown in Table 6b.

TABLE 6b
Soluble carbohydrate and protein of soybean meal generated from null
and transgenic Soil19 event AFS 8377.1.2.1 soybean.

| | Pinitol | Sorbitol | Fructose | Glucose | Sucrose | Galactinol | Raffinose | Stachyose | Total Rafs | Total Carbs | Protein |
|---|---|---|---|---|---|---|---|---|---|---|---|
| Avg. Trans | 0.8 | 0.0 | 0.1 | 0.1 | 9.5 | 0.1 | 0.7 | 0.5 | 1.1 | 11.7 | 58.8 |
| Avg. Null | 0.4 | 0.0 | 0.1 | 0.1 | 6.7 | 0.1 | 1.0 | 6.4 | 7.4 | 14.7 | 54.5 |
| % Change | 121 | | 40 | 68 | 41 | −16 | −35 | −92 | −85 | −21 | 8% |

Table 6b shows an increase in protein in average transgenic Soil19 soybean meal of 8%, an increase in sucrose of 41%, a decrease in raffinosaccharides of 85% and a decrease in total carbohydrates of 21%, when compared to null segregant Soil19 soybean meal.

### Example 7

### Generation of Soybean Lines with Seed-Targeted Silencing of Galactinol Synthase, Fad2, fatB and Fad3 and Seed Targeted Over-Expression of DGAT Enzymes (YLDGAT2 or Gm-DGAT1-09010011)

Fad2-1 b/fatBF/fad3c amiRNA Cassette

The NotI fragment of pKR1776 (SEQ ID NO: 23), containing the 396b-fad2-1b/159-fatBF/159-fad3c triple amiRNA, was cloned into the NotI fragment of pKR1850 (SEQ ID NO: 5), containing the Annexin promoter, to produce pKR1896 (SEQ ID NO: 24).

Stacking Fad2-1 b/fatBF/fad3c amiRNA Cassette with YLDGAT2

The NotI fragment of KS362 (SEQ ID NO: 25), containing the YLDGAT2, was cloned into the NotI site of pKR264 (SEQ ID NO:26) to produce pKR1972 (SEQ ID NO: 27).

The BsiWI fragment of pKR1972 (SEQ ID NO: 27), containing the Gy1/YLDGAT2/leg term cassette, was cloned into the BsiWI site of pKR1896 (SEQ ID NO: 24) to produce pKR2085 (SEQ ID NO: 28).

### Site-Specific Integration Donor Vector

Using standard PCR and cloning methods by one skilled in the art, DNA elements were assembled to produce a 6673 by basic donor construct pKR2008 (SEQ ID NO: 29). Donor construct pKR2008 (SEQ ID NO: 29) is substantially similar to donor plasmid pKR1857 (SEQ ID NO: 14) except that the soybean acetolactate synthase (als) gene coding region encoding a mutant ALS enzyme insensitive to sulfonylurea herbicides comprises a P178S mutation in the encoded protein (previously described in Haughn, G. W., J. Smith, B. Mazur, et al. 1988. Transformation with a mutant Arabidopsis acetolactate synthase gene renders tobacco resistant to sulfonylurea herbicides Molecular and General Genetics MGG February 1988, Volume 211, Issue 2, pp 266-271) rather than the P178A mutation in pKR1857 (SEQ ID NO: 14). In addition, following the ALS gene sequence, the endogenous ALS transcription terminator sequence is utilized instead of the PINII terminator in pKR1857 (SEQ ID NO: 14).

Donor construct pKR2008 (SEQ ID NO: 29) is comprised of the following DNA elements.

Sequence 3989-4036 is a FLP recombinase recognition site FRT1. Sequence 4051-6003 is the soybean acetolactate synthase (als) gene coding region encoding a mutant ALS enzyme insensitive to sulfonylurea herbicides and having a P178S mutation in the encoded protein. Sequence 6007-6657 is the ALS transcription terminator. Sequence 35-50 is a sequence of DNA comprising ORF stop codons in all 6 frames (ORFSTOP-A). Sequence 53-1222 is the phaseolin transcription terminator. Sequence 1254-1270 is a sequence of DNA comprising ORF stop codons in all 6 frames (ORFSTOP-B). Sequence 1343-1390 is a FLP recombinase recognition site FRT87. Sequence 1403-3924 is vector backbone containing the T7 promoter (sequence 2638-2733), the hygromycin phosphotransferase (hpt) gene coding region (sequence 2734-3756) and the T7 terminator (sequence 3781-3913).

Stacking the Fad2-1 b/fatBF/fad3c amiRNA and Galactinol Synthase Silencing Cassettes with YLDGAT2 in an SSI Donor Vector

The AscI fragment of pKR2085 (SEQ ID NO: 28), containing the fad2-1b/fatBF/fad3c amiRNA and YLDGAT2 cassettes, was cloned into the AscI site of construct pKR2008 (SEQ ID NO: 29) to produce pKR2087 (SEQ ID NO: 30).

The SbfI fragment of pKR1292 (SEQ ID NO: 18) was cloned into the SbfI site of pKR2087 (SEQ ID NO: 30) to produce pKR2101 (SEQ ID NO: 31). In this way, YLDGAT2 overexpression and fad2, fatB, fad3 and galactinol synthase gene silencing cassettes were stacked together in one SSI donor construct. Plasmid pKR2101 (SEQ ID NO: 31) was also given the designation PHP52246.

Stacking Fad2-1b/fatBF/fad3c amiRNA Cassette with GM-DGAT1-C9C10C11

Construction of a plasmid pLF179 (SEQ ID NO: 32) containing the modified soy DGAT1 gene (GM-DGAT1-C9C10C11) flanked by NotI sites was previously described (Applicants' Assignee's U.S. Pat. No. 8,101,819; Issued January 24th, 2102).

The NotI fragment of pLF179 (SEQ ID NO: 32), containing the GM-DGAT1-C9C10C11, was cloned into the NotI site of pKR264 (SEQ ID NO:26) to produce pKR1995 (SEQ ID NO: 33).

The BsiWI fragment of pKR1995 (SEQ ID NO: 33), containing the Gy1/GM-DGAT1-09010011/leg term cassette, was cloned into the BsiWI site of pKR1896 (SEQ ID NO: 24) to produce pKR2086 (SEQ ID NO: 34).

Stacking the Fad2-1 b/fatBF/fad3c amiRNA and Galactinol Synthase Silencing Cassettes with GM-DGAT1-09010011 in an SSI Donor Vector

The AscI fragment of pKR2086 (SEQ ID NO: 34), containing the fad2-1b/fatBF/fad3c amiRNA and GM-DGAT1-09010011 cassettes, was cloned into the AscI site of construct pKR2008 (SEQ ID NO: 29) to produce pKR2088 (SEQ ID NO: 35).

The SbfI fragment of pKR1292 (SEQ ID NO: 18) was cloned into the SbfI site of pKR2088 (SEQ ID NO: 35) to produce pKR2102 (SEQ ID NO: 36). In this way, GM-DGAT1-09010011 overexpression and fad2, fatB, fad3 and galactinol synthase gene silencing cassettes were stacked together in one SSI donor construct. Plasmid pKR2102 (SEQ ID NO: 36) was also given the designation PHP52247.

### Example 8

### Generation of Soybean Lines with Seed-Targeted Silencing of Galactinol Synthase, Fad2, fatB and Fad3 and Seed Targeted Over-Expression of DGAT Enzymes (YLDGAT2 or Gm-DGAT1-09010011)

Transformation into Soy SSI Target Events

Target line A cultures were retransformed with the donor construct PHP52246 (SEQ ID NO: 37) and the FLP recombinase construct PHP44664 (SEQ ID NO: 21) using intact plasmid at a 9:3 pg/bp/prep ratio with the biolistic bombardment transformation and events were selected as described elsewhere herein. The experiment name given for this transformation was Soil42.

Target line A cultures were similarly retransformed with the donor construct PHP52247 (SEQ ID NO: 36) and the FLP recombinase construct PHP44664 (SEQ ID NO: 21) using intact plasmid at a 9:3 pg/bp/prep ratio with the biolistic bombardment transformation and events were selected as described elsewhere here in. The experiment name given for this transformation was Soil43.

Soil42 or Soil43 events created through RMCE bring the promoter-less als(P178S) coding region of donor construct PHP52246 (SEQ ID NO: 31) or PHP52247 (SEQ ID NO: 36) downstream of the scp1 promoter of QC288A in target line A for expression and thus chlorsulfuron resistance, respectively.

When the frt1 and frt87 sites from Target line A recombine with those in plasmid PHP52246 (SEQ ID NO: 31) in a successful recombination mediated cassette exchange (RMCE), a new DNA sequence is generated in the genomic DNA as set forth in SEQ ID NO: 37.

When the frt1 and frt87 sites from Target line A recombine with those in plasmid PHP52247 (SEQ ID NO: 36) in a successful recombination mediated cassette exchange (RMCE), a new DNA sequence is generated in the genomic DNA as set forth in SEQ ID NO: 38.

### T0 Embryo and Plant Analysis and Event Selection

Resulting transgenic Soil42 and Soil43 events were selected, maintained and somatic embryos matured as described herein.

Soil42 and Soil43 events were sampled at the somatic embryo stage and screened using construct-specific quantitative PCR (qPCR) as described previously in Assignee's U.S. Pat. No. 8,293,533) issued 2012 Oct. 23 with oligos designed to check for DNA recombination around the FRT1 site and to check for the presence of target, donor, and Flp DNA. Somatic embryos from those Soil42 and Soil43 events that were positive for correct recombination around the FRT1 site were also analyzed for fatty acid profile using GC-FAME and oil content by NMR on ground embryo powder with methods exactly as described herein.

The results for the qPCR, fatty acid and oil analysis of Soil42 and Soil43 events are shown in Table 7 and Table 8, respectively. Based on the qPCR, fatty acid composition and oil content data, events were kept as indicated in Table 7 and 8.

TABLE 7
qPCR, fatty acid composition and oil content of embryos from experiment
Soil42.

| | Fatty Acid Composition | | | | | | | | | | Event |
|---|---|---|---|---|---|---|---|---|---|---|---|
| Soil42 | (wt. %) | | | | | | qPCR Result | | | | Keep |
| Event | 16:0 | 18:0 | 18:1 | 18:2 | 18:3 | % Oil | FRT1 | Donor | Target | FLP | Status |
| AFS | 9.0 | 3.5 | 53.1 | 22.8 | 11.6 | 5.9 | 0.57 | 0.00 | 0.00 | 0.00 | Keep |
| 8720- | | | | | | | | | | | |
| 4-1 | | | | | | | | | | | |
| AFS | 16.6 | 5.1 | 27.7 | 33.1 | 17.4 | 3.3 | 0.88 | 0.00 | 0.00 | 0.00 | Keep |
| 8720- | | | | | | | | | | | |
| 5-1 | | | | | | | | | | | |
| AFS | 8.5 | 3.2 | 43.1 | 31.1 | 14.1 | 3.3 | 0.00 | 1.71 | 0.99 | 1.00 | Throw |
| 8720- | | | | | | | | | | | |
| 5-2 | | | | | | | | | | | |
| AFS | 17.4 | 4.0 | 26.4 | 38.3 | 13.9 | 5.7 | 0.49 | 0.00 | 0.00 | 0.00 | Keep |
| 8720- | | | | | | | | | | | |
| 5-3 | | | | | | | | | | | |
| AFS | 17.7 | 4.2 | 20.1 | 36.4 | 21.7 | 5.8 | 0.31 | 0.00 | 0.00 | 0.00 | Keep |
| 8720- | | | | | | | | | | | |
| 5-4 | | | | | | | | | | | |
| AFS | 4.3 | 2.0 | 48.8 | 35.8 | 9.1 | 4.8 | 0.83 | 0.84 | 0.00 | 0.00 | Keep |
| 8720- | | | | | | | | | | | |
| 6-1 | | | | | | | | | | | |
| AFS | 5.7 | 2.3 | 55.0 | 27.5 | 9.5 | 5.7 | 1.20 | 0.00 | 0.00 | 0.00 | Keep |
| 8720- | | | | | | | | | | | |
| 8-1 | | | | | | | | | | | |
| AFS | 18.0 | 4.2 | 21.3 | 38.6 | 17.9 | 3.2 | 0.68 | 0.42 | 0.81 | 0.83 | Keep |
| 8720- | | | | | | | | | | | |
| 8-2 | | | | | | | | | | | |
| AFS | 11.5 | 3.4 | 35.1 | 36.7 | 13.3 | 3.3 | 1.01 | 0.00 | 0.00 | 0.00 | Keep |
| 8720- | | | | | | | | | | | |
| 8-3 | | | | | | | | | | | |
| AFS | 12.9 | 3.7 | 28.0 | 40.1 | 15.3 | 3.3 | 1.42 | 0.00 | 0.00 | 0.00 | Keep |
| 8720- | | | | | | | | | | | |
| 8-4 | | | | | | | | | | | |
| AFS | 11.7 | 3.8 | 34.4 | 37.4 | 12.8 | 3.4 | 1.27 | 0.00 | 0.00 | 0.00 | Keep |
| 8720- | | | | | | | | | | | |
| 8-5 | | | | | | | | | | | |
| AFS | 8.8 | 2.8 | 47.4 | 29.0 | 12.1 | 4.6 | 1.54 | 0.00 | 0.00 | 0.00 | Keep |
| 8720- | | | | | | | | | | | |
| 8-6 | | | | | | | | | | | |
| AFS | 6.5 | 2.9 | 53.5 | 27.3 | 9.7 | 5.1 | 1.51 | 4.66 | 0.00 | 0.00 | Keep |
| 8720- | | | | | | | | | | | |
| 11-1 | | | | | | | | | | | |
| AFS | 9.2 | 3.7 | 41.7 | 29.8 | 15.6 | 7.0 | 1.08 | 0.00 | 0.00 | 0.00 | Keep |
| 8720- | | | | | | | | | | | |
| 12-1 | | | | | | | | | | | |
| AFS | 15.0 | 3.6 | 21.5 | 41.0 | 18.9 | 4.1 | 0.70 | 4.26 | 0.00 | 0.00 | Keep |
| 8720- | | | | | | | | | | | |
| 12-2 | | | | | | | | | | | |
| AFS | 8.6 | 3.6 | 45.7 | 27.7 | 14.4 | 7.2 | 1.02 | 0.00 | 0.00 | 0.00 | Keep |
| 8720- | | | | | | | | | | | |
| 12-3 | | | | | | | | | | | |

TABLE 8
qPCR, fatty acid composition and oil content of embryos from experiment
Soil43.

| | Fatty Acid Composition | | | | | | | | | | Event |
|---|---|---|---|---|---|---|---|---|---|---|---|
| Soil43 | (wt. %) | | | | | | qPCR Result | | | | Keep |
| Event | 16:0 | 18:0 | 18:1 | 18:2 | 18:3 | % Oil | FRT1 | Donor | Target | FLP | Status |
| AFS | 5.6 | 3.1 | 63.0 | 21.8 | 6.5 | 8.2 | 1.5 | 1.5 | 0.0 | 0.0 | Keep |
| 8738- | | | | | | | | | | | |
| 1-1 | | | | | | | | | | | |
| AFS | 12.2 | 3.7 | 44.0 | 26.4 | 13.6 | 4.9 | 1.8 | 0.0 | 0.0 | 0.0 | Keep |
| 8738- | | | | | | | | | | | |
| 2-1 | | | | | | | | | | | |
| AFS | 14.6 | 6.5 | 33.0 | 34.6 | 11.3 | 4.6 | 1.4 | 1.7 | 0.0 | 0.0 | Keep |
| 8738- | | | | | | | | | | | |
| 6-1 | | | | | | | | | | | |
| AFS | 5.1 | 3.9 | 65.9 | 18.6 | 6.4 | 7.1 | 2.0 | 1.1 | 0.0 | 0.0 | Keep |
| 8738- | | | | | | | | | | | |
| 7-1 | | | | | | | | | | | |
| AFS | 5.6 | 4.1 | 65.3 | 18.5 | 6.5 | 6.4 | 0.0 | 0.0 | 0.0 | 0.0 | Throw |
| 8738- | | | | | | | | | | | |
| 7-2 | | | | | | | | | | | |
| AFS | 3.4 | 2.7 | 71.2 | 19.0 | 3.7 | 9.4 | 2.2 | 8.6 | 0.0 | 0.0 | Keep |
| 8738- | | | | | | | | | | | |
| 7-3 | | | | | | | | | | | |
| AFS | 7.8 | 3.3 | 53.3 | 25.7 | 9.9 | 5.6 | 1.2 | 0.0 | 0.0 | 0.0 | Keep |
| 8738- | | | | | | | | | | | |
| 7-4 | | | | | | | | | | | |
| AFS | 13.4 | 4.9 | 32.3 | 36.5 | 12.9 | 6.2 | 1.5 | 0.0 | 0.0 | 0.0 | Keep |
| 8738- | | | | | | | | | | | |
| 8-1 | | | | | | | | | | | |
| AFS | 5.7 | 3.6 | 65.5 | 20.3 | 4.9 | 8.3 | 1.4 | 0.0 | 1.0 | 1.0 | Keep |
| 8738- | | | | | | | | | | | |
| 8-2 | | | | | | | | | | | |
| AFS | 2.9 | 3.0 | 71.3 | 19.2 | 3.6 | 9.3 | 0.7 | 1.5 | 0.0 | 0.0 | Keep |
| 8738- | | | | | | | | | | | |
| 8-3 | | | | | | | | | | | |
| AFS | 2.9 | 3.1 | 69.7 | 20.8 | 3.4 | 8.6 | 0.0 | 2.4 | 0.0 | 0.0 | Throw |
| 8738- | | | | | | | | | | | |
| 8-4 | | | | | | | | | | | |
| AFS | 2.9 | 2.3 | 68.9 | 22.4 | 3.5 | 7.0 | 0.0 | 3.0 | 0.0 | 0.0 | Throw |
| 8738- | | | | | | | | | | | |
| 8-5 | | | | | | | | | | | |
| AFS | 10.0 | 3.8 | 35.3 | 36.1 | 14.8 | 6.1 | 0.0 | 0.6 | 0.0 | 0.0 | Throw |
| 8738- | | | | | | | | | | | |
| 8-6 | | | | | | | | | | | |
| AFS | 3.3 | 3.1 | 69.8 | 20.3 | 3.5 | 5.8 | 0.0 | 5.7 | 0.0 | 0.0 | Throw |
| 8738- | | | | | | | | | | | |
| 8-7 | | | | | | | | | | | |
| AFS | 6.4 | 3.2 | 54.6 | 27.6 | 8.3 | 5.2 | 1.1 | 0.5 | 0.0 | 0.0 | Keep |
| 8738- | | | | | | | | | | | |
| 8-8 | | | | | | | | | | | |
| AFS | 4.8 | 3.1 | 67.0 | 20.3 | 4.8 | 8.4 | 2.0 | 0.0 | 1.0 | 1.0 | Keep |
| 8738- | | | | | | | | | | | |
| 8-9 | | | | | | | | | | | |
| AFS | 5.3 | 2.9 | 61.6 | 24.1 | 6.0 | 8.2 | 1.9 | 1.8 | 0.0 | 0.0 | Keep |
| 8738- | | | | | | | | | | | |
| 8-10 | | | | | | | | | | | |
| AFS | 6.1 | 3.4 | 57.8 | 26.6 | 6.1 | 8.4 | 0.0 | 13.3 | 1.4 | 1.4 | Throw |
| 8738- | | | | | | | | | | | |
| 9-1 | | | | | | | | | | | |
| AFS | 10.6 | 4.3 | 42.0 | 32.2 | 10.9 | 5.9 | 1.8 | 0.0 | 0.0 | 0.0 | Keep |
| 8738- | | | | | | | | | | | |
| 9-2 | | | | | | | | | | | |
| AFS | 9.8 | 4.3 | 40.5 | 34.8 | 10.7 | 6.9 | 1.5 | 0.0 | 0.0 | 0.0 | Keep |
| 8738- | | | | | | | | | | | |
| 9-3 | | | | | | | | | | | |
| AFS | 3.9 | 3.1 | 66.2 | 21.4 | 5.4 | 7.2 | 0.0 | 6.6 | 0.0 | 0.0 | Throw |
| 8738- | | | | | | | | | | | |
| 9-4 | | | | | | | | | | | |
| AFS | 5.0 | 3.3 | 64.3 | 21.9 | 5.6 | 6.2 | 3.7 | 0.0 | 0.0 | 0.0 | Keep |
| 8738- | | | | | | | | | | | |
| 10-1 | | | | | | | | | | | |
| AFS | 3.0 | 2.9 | 72.7 | 18.5 | 2.9 | 8.2 | 0.0 | 16.1 | 0.0 | 0.0 | Throw |
| 8738- | | | | | | | | | | | |
| 10-2 | | | | | | | | | | | |
| AFS | 7.7 | 3.8 | 55.6 | 23.9 | 9.1 | 4.1 | 1.3 | 0.0 | 0.0 | 0.0 | Keep |
| 8738- | | | | | | | | | | | |
| 11-1 | | | | | | | | | | | |
| AFS | 7.1 | 3.4 | 50.6 | 27.6 | 11.3 | 3.9 | 1.0 | 0.0 | 1.3 | 1.3 | Keep |
| 8738- | | | | | | | | | | | |
| 11-2 | | | | | | | | | | | |
| AFS | 5.5 | 3.5 | 63.8 | 21.2 | 6.0 | 6.4 | 1.6 | 0.0 | 0.0 | 0.0 | Keep |
| 8738- | | | | | | | | | | | |
| 11-3 | | | | | | | | | | | |
| AFS | 6.8 | 3.5 | 55.8 | 26.3 | 7.7 | 6.3 | 1.1 | 0.0 | 0.0 | 0.0 | Keep |
| 8738- | | | | | | | | | | | |
| 12-1 | | | | | | | | | | | |
| AFS | 7.3 | 3.7 | 55.0 | 24.9 | 9.1 | 6.1 | 2.1 | 0.0 | 0.0 | 0.0 | Keep |
| 8738- | | | | | | | | | | | |
| 12-2 | | | | | | | | | | | |

Somatic embryos from kept Soil42 and Soil43 events were dried, germinated and planted and resulting T0 plants were grown as described herein.

### T1 Seed Oil Content and Fatty Acid Composition Analysis

Oil content of T1 seed from Soil42 events AFS 8720-8-6 and AFS 8720-12-3 and Soil43 event AFS 8738-11-2 was determined by NMR as described herein. A small seed chip was taken from each T1 seed from each event, seed lipid was hexane extracted and the fatty acid composition determined by GC-FAME as described herein. The remaining seed chip was extracted with methanol and soluble sugars separated and visualized by TLC as described herein (Example 29). The results for oil content, fatty acid profile and sugar composition by TLC for the Soil42 events is shown in Table 9 and for Soil43 in Table 10.

In Table 9 and 10, oil content is the weight percent oil of total seed weight and the fatty acid profile is the weight percent for individual fatty acids of total fatty acid. The amount of sucrose increase and stachyose decrease as indicated by the TLC plate is scored on a scale of 0-3 where a 0 indicates wild-type levels of sugar and a 3 indicates substantially reduced stachyose and substantially increased sucrose. When a line cell is left blank, the oil, fatty acid profile or TLC score of a seed chip was not determined. In Table 9 and 10, results for each event are divided according to transgenic and null based on the TLC result and/or the oleic acid and total saturated fatty acids (Total Sats) content. Results are then sorted based on oil content. The average value for transgenic or null is indicated at the bottom of each column. A column indicating seed chosen for further compositional analysis is also shown in Table 9 and 10.

TABLE 9
Fatty acid composition, sugar readout and oil content of T1 Seed from
experiment Soil42.

| | | | | | | | | | Chosen |
|---|---|---|---|---|---|---|---|---|---|
| | | | | | | | | Total | for |
| T1 Seed | TLC | % Oil | 16:0% | 18:0% | 18:1% | 18:2% | 18:3% | Sats | Comp |
| AFS | 3 | 24.2 | 3.0 | 3.3 | 82.8 | 5.7 | 5.1 | 6.3 | 1 |
| 8720.8.6.3.34 | | | | | | | | | |
| AFS | 3 | 24.4 | 2.7 | 3.3 | 84.8 | 5.0 | 4.1 | 6.0 | 1 |
| 8720.8.6.3.5 | | | | | | | | | |
| AFS | 3 | 23.4 | 3.3 | 2.7 | 85.4 | 4.3 | 4.3 | 6.0 | 1 |
| 8720.8.6.3.15 | | | | | | | | | |
| AFS | 3 | 23.5 | 3.2 | 3.0 | 82.6 | 5.6 | 5.6 | 6.2 | 1 |
| 8720.8.6.3.14 | | | | | | | | | |
| AFS | 3 | 24.0 | 2.3 | 3.1 | 88.6 | 3.0 | 3.0 | 5.4 | 1 |
| 8720.8.6.3.3 | | | | | | | | | |
| AFS | 3 | 23.6 | 3.4 | 3.4 | 80.4 | 5.9 | 6.8 | 6.9 | |
| 8720.8.6.3.26 | | | | | | | | | |
| AFS | 3 | 22.8 | 3.4 | 3.1 | 77.7 | 7.9 | 7.9 | 6.5 | |
| 8720.8.6.3.30 | | | | | | | | | |
| AFS | 3 | 22.8 | 3.6 | 3.3 | 77.7 | 8.2 | 7.3 | 6.9 | |
| 8720.8.6.3.24 | | | | | | | | | |
| AFS | 3 | 22.8 | 2.6 | 2.8 | 85.7 | 4.2 | 4.7 | 5.4 | |
| 8720.8.6.3.8 | | | | | | | | | |
| AFS | 3 | 22.4 | 3.2 | 3.5 | 81.3 | 6.3 | 5.6 | 6.8 | |
| 8720.8.6.3.21 | | | | | | | | | |
| AFS | 3 | 22.2 | 2.7 | 3.4 | 84.4 | 5.0 | 4.6 | 6.1 | |
| 8720.8.6.3.18 | | | | | | | | | |
| AFS | 3 | 22.0 | 3.7 | 3.1 | 79.4 | 7.4 | 6.4 | 6.8 | |
| 8720.8.6.3.31 | | | | | | | | | |
| AFS | 3 | 21.5 | 2.1 | 2.9 | 85.3 | 4.3 | 5.4 | 5.0 | |
| 8720.8.6.3.28 | | | | | | | | | |
| AFS | 3 | 22.3 | 2.3 | 3.9 | 84.2 | 4.2 | 5.4 | 6.2 | |
| 8720.8.6.3.32 | | | | | | | | | |
| AFS | 3 | 21.6 | 3.7 | 3.1 | 78.2 | 8.4 | 6.6 | 6.8 | |
| 8720.8.6.3.4 | | | | | | | | | |
| AFS | 3 | 21.7 | 4.0 | 3.1 | 74.2 | 9.4 | 9.2 | 7.2 | |
| 8720.8.6.3.12 | | | | | | | | | |
| AFS | 3 | 21.3 | 3.7 | 4.3 | 76.0 | 8.7 | 7.3 | 8.0 | |
| 8720.8.6.3.1 | | | | | | | | | |
| AFS | 3 | 21.4 | 2.9 | 3.3 | 82.1 | 6.0 | 5.6 | 6.3 | |
| 8720.8.6.3.17 | | | | | | | | | |
| AFS | 3 | 21.4 | 4.0 | 3.3 | 75.7 | 10.0 | 7.0 | 7.3 | |
| 8720.8.6.3.2 | | | | | | | | | |
| AFS | 3 | 21.1 | 2.5 | 3.6 | 85.2 | 5.0 | 3.7 | 6.1 | |
| 8720.8.6.3.33 | | | | | | | | | |
| AFS | 3 | 20.9 | 3.9 | 3.3 | 75.6 | 9.9 | 7.4 | 7.1 | |
| 8720.8.6.3.23 | | | | | | | | | |
| AFS | 3 | 20.9 | 3.4 | 3.1 | 77.3 | 8.9 | 7.3 | 6.5 | |
| 8720.8.6.3.36 | | | | | | | | | |
| AFS | 3 | 20.7 | 2.7 | 3.2 | 85.1 | 4.6 | 4.5 | 5.9 | |
| 8720.8.6.3.20 | | | | | | | | | |
| AFS | 3 | 20.5 | 3.7 | 3.3 | 78.1 | 8.5 | 6.4 | 6.9 | |
| 8720.8.6.3.9 | | | | | | | | | |
| AFS | 3 | 19.7 | 3.6 | 2.9 | 76.7 | 7.2 | 9.6 | 6.5 | |
| 8720.8.6.3.19 | | | | | | | | | |
| AFS | 3 | 19.5 | 3.6 | 3.2 | 76.0 | 9.6 | 7.6 | 6.8 | |
| 8720.8.6.3.7 | | | | | | | | | |
| AFS | 3 | 19.0 | 3.5 | 3.4 | 75.6 | 11.0 | 6.6 | 6.8 | |
| 8720.8.6.3.16 | | | | | | | | | |
| AFS | 3 | 17.8 | 3.8 | 3.4 | 75.9 | 9.0 | 7.9 | 7.2 | |
| 8720.8.6.3.11 | | | | | | | | | |
| Trans Avg. | | 21.8 | 3.2 | 3.3 | 80.4 | 6.9 | 6.2 | 6.5 | |
| AFS | 0 | 22.9 | 10.0 | 3.7 | 13.0 | 57.6 | 15.8 | 13.6 | 1 |
| 8720.8.6.3.22 | | | | | | | | | |
| AFS | 0 | 20.8 | 10.4 | 3.7 | 26.7 | 46.0 | 13.2 | 14.1 | 1 |
| 8720.8.6.3.10 | | | | | | | | | |
| AFS | 0 | 20.8 | 11.2 | 4.5 | 19.4 | 52.4 | 12.5 | 15.7 | 1 |
| 8720.8.6.3.25 | | | | | | | | | |
| AFS | 0 | 21.0 | 11.0 | 4.1 | 14.1 | 53.6 | 17.2 | 15.1 | 1 |
| 8720.8.6.3.6 | | | | | | | | | |
| AFS | 0 | 20.1 | 12.4 | 4.1 | 15.3 | 54.1 | 14.1 | 16.5 | 1 |
| 8720.8.6.3.35 | | | | | | | | | |
| AFS | 0 | 20.3 | 12.3 | 3.8 | 16.8 | 52.2 | 14.9 | 16.1 | |
| 8720.8.6.3.29 | | | | | | | | | |
| AFS | 0 | 19.3 | 11.6 | 4.9 | 14.4 | 54.9 | 14.2 | 16.5 | |
| 8720.8.6.3.27 | | | | | | | | | |
| Null Avg. | | 20.7 | 11.3 | 4.1 | 17.1 | 53.0 | 14.6 | 15.4 | |
| AFS | 3 | 25.0 | 2.2 | 2.4 | 86.2 | 4.0 | 5.1 | 4.7 | 1 |
| 8720.12.3.1.3 | | | | | | | | | |
| AFS | 3 | 23.1 | 3.4 | 3.2 | 86.3 | 3.7 | 3.4 | 6.6 | 1 |
| 8720.12.3.1.24 | | | | | | | | | |
| AFS | 3 | 22.6 | 2.6 | 3.0 | 84.5 | 4.4 | 5.4 | 5.7 | 1 |
| 8720.12.3.1.28 | | | | | | | | | |
| AFS | 3 | 23.0 | 2.8 | 2.7 | 86.1 | 4.0 | 4.5 | 5.5 | 1 |
| 8720.12.3.1.19 | | | | | | | | | |
| AFS | 3 | 22.7 | 2.8 | 3.3 | 84.9 | 4.9 | 4.1 | 6.1 | 1 |
| 8720.12.3.1.15 | | | | | | | | | |
| AFS | 3 | 22.6 | 2.7 | 3.4 | 81.6 | 6.2 | 6.1 | 6.1 | |
| 8720.12.3.1.11 | | | | | | | | | |
| AFS | 3 | 22.1 | 2.8 | 2.9 | 84.8 | 4.2 | 5.3 | 5.6 | |
| 8720.12.3.1.8 | | | | | | | | | |
| AFS | 3 | 21.8 | 2.8 | 3.0 | 84.1 | 4.9 | 5.2 | 5.8 | |
| 8720.12.3.1.10 | | | | | | | | | |
| AFS | 3 | 21.9 | 3.1 | 3.0 | 82.4 | 6.3 | 5.2 | 6.1 | |
| 8720.12.3.1.13 | | | | | | | | | |
| AFS | 3 | 21.8 | 3.1 | 2.7 | 84.7 | 4.8 | 4.8 | 5.8 | |
| 8720.12.3.1.6 | | | | | | | | | |
| AFS | 3 | 21.3 | 3.0 | 2.8 | 86.3 | 4.3 | 3.6 | 5.7 | |
| 8720.12.3.1.36 | | | | | | | | | |
| AFS | 3 | 21.7 | 2.7 | 2.7 | 87.2 | 3.4 | 4.0 | 5.4 | |
| 8720.12.3.1.29 | | | | | | | | | |
| AFS | 3 | 21.0 | 2.7 | 3.0 | 84.1 | 6.6 | 3.7 | 5.7 | |
| 8720.12.3.1.26 | | | | | | | | | |
| AFS | 3 | 20.9 | 3.1 | 3.0 | 81.4 | 6.4 | 6.0 | 6.2 | |
| 8720.12.3.1.4 | | | | | | | | | |
| AFS | 3 | 21.6 | 2.8 | 2.7 | 85.3 | 4.5 | 4.7 | 5.5 | |
| 8720.12.3.1.7 | | | | | | | | | |
| AFS | 3 | 21.0 | 2.7 | 3.3 | 83.5 | 5.5 | 5.1 | 6.0 | |
| 8720.12.3.1.32 | | | | | | | | | |
| AFS | 3 | 20.8 | 2.8 | 3.0 | 84.3 | 4.8 | 5.1 | 5.8 | |
| 8720.12.3.1.33 | | | | | | | | | |
| AFS | 3 | 21.2 | 3.3 | 3.5 | 83.3 | 5.0 | 5.0 | 6.7 | |
| 8720.12.3.1.27 | | | | | | | | | |
| AFS | 3 | 21.0 | 3.0 | 2.8 | 84.1 | 4.6 | 5.5 | 5.8 | |
| 8720.12.3.1.21 | | | | | | | | | |
| AFS | 3 | 20.6 | 3.0 | 2.9 | 83.7 | 4.9 | 5.6 | 5.9 | |
| 8720.12.3.1.5 | | | | | | | | | |
| AFS | 3 | 20.5 | 3.2 | 2.8 | 83.9 | 4.9 | 5.1 | 6.0 | |
| 8720.12.3.1.18 | | | | | | | | | |
| AFS | 3 | 20.9 | 2.8 | 3.4 | 81.1 | 7.1 | 5.6 | 6.2 | |
| 8720.12.3.1.2 | | | | | | | | | |
| AFS | 3 | 21.0 | 2.8 | 3.1 | 86.7 | 3.6 | 3.8 | 5.9 | |
| 8720.12.3.1.35 | | | | | | | | | |
| AFS | 3 | 20.6 | 2.6 | 3.3 | 84.6 | 4.6 | 4.9 | 5.9 | |
| 8720.12.3.1.34 | | | | | | | | | |
| AFS | 3 | 19.2 | 2.9 | 2.7 | 84.3 | 5.0 | 5.2 | 5.5 | |
| 8720.12.3.1.25 | | | | | | | | | |
| Trans Avg. | | 21.6 | 2.9 | 3.0 | 84.4 | 4.9 | 4.9 | 5.9 | |
| AFS | 0 | 24.0 | 11.5 | 4.1 | 19.7 | 52.3 | 12.3 | 15.6 | 1 |
| 8720.12.3.1.9 | | | | | | | | | |
| AFS | 0 | 21.4 | 10.8 | 3.8 | 17.1 | 55.1 | 13.2 | 14.6 | 1 |
| 8720.12.3.1.16 | | | | | | | | | |
| AFS | 0 | 20.8 | 10.9 | 4.1 | 16.6 | 56.0 | 12.5 | 14.9 | 1 |
| 8720.12.3.1.17 | | | | | | | | | |
| AFS | 0 | 21.0 | 11.3 | 3.6 | 16.0 | 55.5 | 13.6 | 14.9 | 1 |
| 8720.12.3.1.23 | | | | | | | | | |
| AFS | 0 | 20.5 | 11.1 | 4.1 | 22.1 | 50.5 | 12.3 | 15.2 | 1 |
| 8720.12.3.1.12 | | | | | | | | | |
| AFS | 0 | 20.5 | 11.5 | 3.6 | 17.2 | 55.1 | 12.6 | 15.1 | |
| 8720.12.3.1.31 | | | | | | | | | |
| AFS | 0 | 20.4 | 11.6 | 4.4 | 14.8 | 55.5 | 13.7 | 16.0 | |
| 8720.12.3.1.14 | | | | | | | | | |
| AFS | 0 | 20.3 | 11.2 | 4.4 | 12.9 | 53.0 | 18.5 | 15.5 | |
| 8720.12.3.1.1 | | | | | | | | | |
| AFS | 0 | 19.5 | 9.9 | 3.6 | 15.7 | 55.8 | 15.0 | 13.5 | |
| 8720.12.3.1.20 | | | | | | | | | |
| AFS | 0 | 19.3 | 11.6 | 3.8 | 14.7 | 55.2 | 14.7 | 15.4 | |
| 8720.12.3.1.30 | | | | | | | | | |
| AFS | 0 | 18.0 | 12.3 | 3.6 | 13.6 | 52.2 | 18.3 | 15.8 | |
| 8720.12.3.1.22 | | | | | | | | | |
| Null Avg. | | 20.5 | 11.2 | 3.9 | 16.4 | 54.2 | 14.2 | 15.2 | |

TABLE 10
Fatty acid composition, sugar readout and oil content of T1 Seed from
experiment Soil43.

| | | | | | | | | | Chosen |
|---|---|---|---|---|---|---|---|---|---|
| | | | | | | | | Total | for |
| T1 Seed | TLC | % Oil | 16:0% | 18:0% | 18:1% | 18:2% | 18:3% | Sats | Comp |
| AFS8738.11.2.4.14 | 3 | 25.2 | 2.4 | 4.0 | 85.9 | 4.0 | 3.7 | 6.4 | 1 |
| AFS8738.11.2.4.36 | 3 | 23.9 | 2.3 | 3.3 | 86.9 | 3.6 | 3.9 | 5.6 | 1 |
| AFS8738.11.2.4.23 | 3 | 24.1 | 2.3 | 2.8 | 85.0 | 4.3 | 5.6 | 5.1 | 1 |
| AFS8738.11.2.4.5 | 3 | 22.2 | 2.5 | 3.7 | 86.0 | 3.6 | 4.3 | 6.2 | 1 |
| AFS8738.11.2.4.26 | 3 | 22.2 | 3.2 | 3.4 | 80.2 | 7.5 | 5.7 | 6.6 | 1 |
| AFS8738.11.2.4.28 | 3 | 22.3 | 2.2 | 2.9 | 87.5 | 3.4 | 4.0 | 5.1 | |
| AFS8738.11.2.4.18 | 3 | 22.2 | 2.6 | 3.0 | 85.6 | 3.8 | 5.0 | 5.6 | |
| AFS8738.11.2.4.4 | 3 | 22.1 | 2.4 | 2.8 | 86.9 | 3.1 | 4.8 | 5.2 | |
| AFS8738.11.2.4.8 | 3 | 22.0 | 1.8 | 2.4 | 89.3 | 2.8 | 3.7 | 4.2 | |
| AFS8738.11.2.4.24 | 3 | 21.9 | 2.4 | 2.9 | 86.7 | 3.3 | 4.7 | 5.3 | |
| AFS8738.11.2.4.30 | 3 | 21.8 | 2.7 | 3.3 | 86.0 | 3.9 | 4.1 | 6.0 | |
| AFS8738.11.2.4.22 | 3 | 21.7 | 2.5 | 2.8 | 86.9 | 3.6 | 4.2 | 5.3 | |
| AFS8738.11.2.4.2 | 3 | 21.7 | 2.7 | 3.0 | 86.2 | 3.6 | 4.5 | 5.7 | |
| AFS8738.11.2.4.33 | 3 | 21.6 | 2.6 | 2.9 | 85.7 | 4.7 | 4.0 | 5.5 | |
| AFS8738.11.2.4.17 | 3 | 21.2 | 3.1 | 3.1 | 80.7 | 7.5 | 5.6 | 6.2 | |
| AFS8738.11.2.4.6 | 3 | 21.1 | 2.5 | 2.6 | 86.2 | 3.5 | 5.1 | 5.1 | |
| AFS8738.11.2.4.15 | 3 | 21.0 | 2.7 | 2.9 | 84.3 | 4.6 | 5.4 | 5.7 | |
| AFS8738.11.2.4.12 | 3 | 21.0 | 2.6 | 2.6 | 86.5 | 3.6 | 4.8 | 5.2 | |
| AFS8738.11.2.4.7 | 3 | 20.9 | 2.5 | 2.7 | 85.2 | 3.9 | 5.7 | 5.1 | |
| AFS8738.11.2.4.21 | 3 | 20.6 | 2.6 | 2.9 | 85.1 | 4.0 | 5.3 | 5.5 | |
| AFS8738.11.2.4.9 | 3 | 20.6 | 2.8 | 2.7 | 85.6 | 4.0 | 4.9 | 5.5 | |
| AFS8738.11.2.4.34 | 3 | 20.5 | 2.5 | 3.3 | 85.2 | 4.1 | 4.9 | 5.8 | |
| AFS8738.11.2.4.20 | 3 | 20.5 | 2.4 | 2.9 | 86.6 | 4.5 | 3.6 | 5.3 | |
| AFS8738.11.2.4.27 | 3 | 20.5 | 2.6 | 3.1 | 84.6 | 4.7 | 5.0 | 5.7 | |
| AFS8738.11.2.4.29 | 3 | 20.3 | 2.6 | 2.7 | 86.2 | 3.5 | 5.0 | 5.3 | |
| AFS8738.11.2.4.13 | 3 | 20.1 | 2.0 | 2.3 | 89.8 | 2.5 | 3.5 | 4.3 | |
| AFS8738.11.2.4.35 | 3 | 18.7 | 2.5 | 3.6 | 85.4 | 4.3 | 4.2 | 6.1 | |
| AFS8738.11.2.4.10 | 3 | 18.6 | 1.9 | 2.3 | 89.1 | 2.8 | 3.9 | 4.3 | |
| AFS8738.11.2.4.11 | 3 | 18.5 | 1.8 | 2.2 | 88.3 | 3.3 | 4.3 | 4.1 | |
| Trans | | 21.3 | 2.5 | 2.9 | 86.0 | 4.0 | 4.6 | 5.4 | |
| Avg. | | | | | | | | | |
| AFS8738.11.2.4.32 | 0 | 21.9 | 11.3 | 4.0 | 16.7 | 53.2 | 14.8 | 15.3 | 1 |
| AFS8738.11.2.4.31 | 0 | 20.6 | 10.8 | 3.8 | 22.2 | 50.1 | 13.2 | 14.6 | 1 |
| AFS8738.11.2.4.25 | 0 | 19.6 | 12.1 | 3.8 | 17.1 | 52.1 | 14.9 | 15.9 | 1 |
| AFS8738.11.2.4.19 | 1 | 19.0 | 11.1 | 3.6 | 18.1 | 53.2 | 13.9 | 14.7 | 1 |
| AFS8738.11.2.4.16 | 0 | 18.6 | 10.9 | 3.8 | 19.6 | 50.7 | 15.0 | 14.7 | 1 |
| Null Avg. | | 19.9 | 11.2 | 3.8 | 18.7 | 51.9 | 14.3 | 15.0 | |

From Tables 9 and 10, it can be seen that transgenic seed have higher oil contents than null seed and also having higher oleic acid, lower saturated fatty acids and lower alpha-linolenic contents compared to null seed. Also, transgenic seed show decreased raffinosaccharides and increased sucrose by TLC compared to null seed.

Additionally, it can be seen that all events segregate for phenotype (TLC result and fatty acid profile changes) in a 3:1 Mendelian fashion.

A summary of the average values for oil content and fatty acid profile for T1 seed from each event from Soil42 and Soil43, along with the difference in oil content for each event is shown in Table 11. In Table 11, the percent change of transgenic compared to null is indicated as % Change.

TABLE 11
Average oil contents and fatty acid profiles for T1 seed from RMCE events from Soil42 and Soil43.

| | | T0 | T1 | | | | | | | Total |
|---|---|---|---|---|---|---|---|---|---|---|
| Experiment | Event | Plant | Seed | % Oil | 16:0% | 18:0% | 18:1% | 18:2% | 18:3% | Sats |
| Soil42 | AFS | AFS | Trans Avg. | 21.8 | 3.2 | 3.3 | 80.4 | 6.9 | 6.2 | 6.5 |
| | 8720-8-6 | 8720.8.6.3 | | | | | | | | |
| Soil42 | AFS | AFS | Null Avg. | 20.7 | 11.3 | 4.1 | 17.1 | 53.0 | 14.6 | 15.4 |
| | 8720-8-6 | 8720.8.6.3 | | | | | | | | |
| | | | % Change | 5 | −71 | −21 | 370 | −87 | −58 | −58 |
| Soil42 | AFS | AFS | Trans Avg. | 21.6 | 2.9 | 3.0 | 84.4 | 4.9 | 4.9 | 5.9 |
| | 8720-12-3 | 8720.12.3.1 | | | | | | | | |
| Soil42 | AFS | AFS | Null Avg. | 20.5 | 11.2 | 3.9 | 16.4 | 54.2 | 14.2 | 15.2 |
| | 8720-12-3 | 8720.12.3.1 | | | | | | | | |
| | | | % Change | 5 | −74 | −24 | 414 | −91 | −66 | −61 |
| Soil43 | AFS | AFS | Trans Avg. | 21.3 | 2.5 | 2.9 | 86.0 | 4.0 | 4.6 | 5.4 |
| | 8738-11-2 | 8738.11.2.4 | | | | | | | | |
| Soil43 | AFS | AFS | Null Avg. | 19.9 | 11.2 | 3.8 | 18.7 | 51.9 | 14.3 | 15.0 |
| | 8738-11-2 | 8738.11.2.4 | | | | | | | | |
| | | | % Change | 7 | −78 | −23 | 359 | −92 | −68 | −64 |

In Table 11, average oil content percent increases comparing all transgenic seed to null segregant T1 seed range from 5 to 7% over null. Palmitic acid average percent decrease ranges from 71 to 78%. Stearic acid average percent decrease ranges from 21 to 24%. Total saturated fatty acid average percent decrease ranges from 58 to 64%. Oleic acid average percent increase ranges from 359 to 414%. Linoleic acid average percent decrease ranges from 87 to 92%. Alpha-linolenic acid average percent decrease ranges from 58 to 68%.

Compositional Analysis of T1 Seed from Soil42 and Soil43 Events

Five individual transgenic and corresponding null T1 seed from Soil42 events and Soil43 events (indicated in Tables 9 and 10 as Chosen for comp) were each ground to a powder using the genogrinder as described herein.

Non-structural soluble carbohydrate and protein from the chosen Soil42 and Soil43 T1 seed transgenic and null powders were quantified using the methods described in Example 2 and oil content of powders was determined by NMR as described herein and results are presented in Table 12. Total lipids extracted from dry powders using heptane extraction were analyzed for fatty acid composition by derivatization with TMSH and GC-FAME as described herein is also presented for each seed in Table 12.

In Table 12, individual soluble carbohydrates (pinitol, sorbitol, fructose, glucose, sucrose, galactinol, raffinose, stachyose) as well as protein and oil are for individual seed are reported as a percent of ground soy powder. Also presented are the total rafinosaccharides (Total Rafs; sum of raffinose and stachyose) and total soluble carbohydrates (Total Carbs; sum of individual carbohydrates).

In Table 12, the average value for all transgenic seed or null seed for each individual event is shown (Avg.).

TABLE 12
Soluble carbohydrate, protein and oil content of individual
seed from null and transgenic Soil42 event and Soil43 events.

| | T1 | Pini- | Sorbi- | Fruc- | Glu- | Su- | Galac- | Raffi- | Stach- | Total | Total | Pro- | |
|---|---|---|---|---|---|---|---|---|---|---|---|---|---|
| Exp | Seed ¹ | tol | tol | tose | cose | crose | tinol | nose | yose | Rafs | Carbs | tein | Oil |
| Soil42 | 3 | 0.00 | 0.02 | 0.13 | 0.02 | 7.43 | 0.18 | 0.44 | 0.00 | 0.44 | 8.21 | 37.0 | 23.2 |
| Soil42 | 15 | 0.00 | 0.02 | 0.04 | 0.04 | 6.82 | 0.03 | 0.75 | 0.31 | 1.07 | 8.02 | 38.9 | 22.4 |
| Soil42 | 5 | 0.00 | 0.00 | 0.04 | 0.00 | 5.17 | 0.21 | 0.46 | 0.00 | 0.46 | 5.88 | 35.9 | 23.1 |
| Soil42 | 34 | 0.00 | 0.02 | 0.05 | 0.05 | 7.10 | 0.03 | 0.72 | 0.33 | 1.04 | 8.28 | 37.1 | 23.9 |
| Soil42 | 14 | 0.00 | 0.00 | 0.04 | 0.04 | 7.15 | 0.02 | 0.62 | 0.30 | 0.92 | 8.17 | 39.5 | 22.8 |
| | Trans. Avg | 0.00 | 0.01 | 0.06 | 0.03 | 6.73 | 0.09 | 0.60 | 0.19 | 0.79 | 7.71 | 37.7 | 23.1 |
| Soil42 | 10 | 0.00 | 0.01 | 0.04 | 0.04 | 6.37 | 0.02 | 0.93 | 2.49 | 3.42 | 9.90 | 37.4 | 19.3 |
| Soil42 | 25 | 0.00 | 0.02 | 0.05 | 0.04 | 6.70 | 0.02 | 0.85 | 2.21 | 3.06 | 9.89 | 36.1 | 20.2 |
| Soil42 | 35 | 0.00 | 0.02 | 0.05 | 0.05 | 7.05 | 0.03 | 1.12 | 2.80 | 3.91 | 11.1 | 35.7 | 19.0 |
| Soil42 | 6 | 0.00 | 0.02 | 0.04 | 0.03 | 6.16 | 0.02 | 0.91 | 2.79 | 3.70 | 9.96 | 37.9 | 20.4 |
| Soil42 | 22 | 0.00 | 0.02 | 0.04 | 0.04 | 5.82 | 0.03 | 1.08 | 2.80 | 3.87 | 9.82 | 39.6 | 22.3 |
| | Null Avg | 0.00 | 0.02 | 0.04 | 0.04 | 6.42 | 0.02 | 0.98 | 2.62 | 3.59 | 10.1 | 37.3 | 20.2 |
| Soil42 | 24 | 0.00 | 0.02 | 0.05 | 0.04 | 7.84 | 0.02 | 0.87 | 0.43 | 1.30 | 9.26 | 37.1 | 22.3 |
| Soil42 | 3 | 0.00 | 0.02 | 0.03 | 0.04 | 7.37 | 0.02 | 0.72 | 0.41 | 1.13 | 8.61 | 33.8 | 23.5 |
| Soil42 | 19 | 0.00 | 0.02 | 0.06 | 0.05 | 9.01 | 0.02 | 0.73 | 0.35 | 1.08 | 10.2 | 38.9 | 21.9 |
| Soil42 | 15 | 0.00 | 0.03 | 0.04 | 0.05 | 8.60 | 0.02 | 1.22 | 0.44 | 1.66 | 10.4 | 34.2 | 21.7 |
| Soil42 | 28 | 0.00 | 0.02 | 0.08 | 0.06 | 8.71 | 0.18 | 0.81 | 0.36 | 1.16 | 10.2 | 37.6 | 22.0 |
| | Trans. Avg. | 0.00 | 0.02 | 0.05 | 0.05 | 8.31 | 0.05 | 0.87 | 0.40 | 1.27 | 9.74 | 36.3 | 22.3 |
| Soil42 | 12 | 0.00 | 0.02 | 0.03 | 0.02 | 5.13 | 0.02 | 0.89 | 3.34 | 4.24 | 9.47 | 37.3 | 19.6 |
| Soil42 | 9 | 0.00 | 0.02 | 0.03 | 0.03 | 6.05 | 0.06 | 0.58 | 3.53 | 4.11 | 10.3 | 32.0 | 22.8 |
| Soil42 | 16 | 0.00 | 0.02 | 0.03 | 0.03 | 4.77 | 0.06 | 0.52 | 2.69 | 3.21 | 8.11 | 36.2 | 20.6 |
| Soil42 | 17 | 0.00 | 0.03 | 0.05 | 0.04 | 7.89 | 0.02 | 1.27 | 2.47 | 3.74 | 11.8 | 36.3 | 20.1 |
| Soil42 | 23 | 0.00 | 0.03 | 0.06 | 0.04 | 8.11 | 0.03 | 1.33 | 2.84 | 4.17 | 12.4 | 33.3 | 20.4 |
| | Null Avg. | 0.00 | 0.02 | 0.04 | 0.03 | 6.39 | 0.04 | 0.92 | 2.97 | 3.89 | 10.4 | 35.0 | 20.7 |
| Soil43 | 36 | 0.02 | 0.03 | 0.14 | 0.18 | 6.99 | 0.03 | 0.90 | 1.19 | 2.10 | 9.49 | 35.3 | 23.0 |
| Soil43 | 5 | 0.02 | 0.02 | 0.15 | 0.19 | 7.99 | 0.02 | 0.68 | 0.64 | 1.32 | 9.71 | 38.4 | 20.4 |
| Soil43 | 14 | 0.02 | 0.02 | 0.13 | 0.20 | 7.55 | 0.04 | 0.77 | 0.79 | 1.56 | 9.51 | 31.3 | 24.1 |
| Soil43 | 23 | 0.02 | 0.02 | 0.12 | 0.17 | 6.68 | 0.02 | 0.74 | 0.72 | 1.46 | 8.50 | 33.5 | 22.2 |
| Soil43 | 26 | 0.02 | 0.02 | 0.14 | 0.18 | 7.52 | 0.02 | 0.63 | 0.64 | 1.27 | 9.18 | 36.3 | 21.4 |
| | Trans. Avg. | 0.02 | 0.02 | 0.14 | 0.18 | 7.34 | 0.03 | 0.74 | 0.80 | 1.54 | 9.28 | 35.0 | 22.2 |
| Soil43 | 31 | 0.02 | 0.02 | 0.14 | 0.15 | 6.45 | 0.04 | 1.15 | 2.66 | 3.80 | 10.6 | 35.4 | 19.8 |
| Soil43 | 16 | 0.02 | 0.02 | 0.13 | 0.15 | 6.05 | 0.04 | 0.77 | 3.06 | 3.84 | 10.2 | 34.9 | 17.6 |
| Soil43 | 19 | 0.02 | 0.02 | 0.15 | 0.15 | 6.72 | 0.03 | 1.27 | 2.54 | 3.82 | 10.9 | 35.8 | 18.1 |
| Soil43 | 25 | 0.02 | 0.02 | 0.13 | 0.14 | 5.54 | 0.04 | 0.80 | 3.20 | 3.99 | 9.88 | 36.3 | 19.2 |
| Soil43 | 32 | 0.02 | 0.02 | 0.11 | 0.12 | 4.95 | 0.03 | 0.54 | 3.02 | 3.57 | 8.82 | 35.1 | 21.0 |
| | Null Avg. | 0.02 | 0.02 | 0.13 | 0.14 | 5.94 | 0.03 | 0.91 | 2.90 | 3.80 | 10.1 | 35.5 | 19.1 |

¹ For experiment Soil42 (AFS8720.8.6) seeds come from T0 plants AFS 8720.8.6.3 (numerical rows 1-12) or from T0 Plants AFS 8720.12.3.1 (numerical rows 13-24. For experiment Soil43 (AFS 8738.11.2) seeds come from T0 plants AFS 8738.11.2.4.

The average values for all transgenic seed or null seed (Avg.) for individual soluble carbohydrates (pinitol, sorbitol, fructose, glucose, sucrose, galactinol, raffinose, stachyose) as well as protein and oil are summarized in Table 13. Also presented are the average values for total rafinosaccharides (Total Rafs; sum of raffinose and stachyose) and total soluble carbohydrates (Total Carbs; sum of individual carbohydrates).

Additionally, the percent increase or decrease (percent change) for the average of any particular soluble carbohydrate, protein or oil as is also shown in Table 13 where the percent is calculated in the following way; [(transgenic value-null value)/null value×100%].

TABLE 13
Average soluble carbohydrate, protein and oil content of individual
seed from null and transgenic Soil42 event and Soil43 events.

| | | Pini- | Sorbi- | Fruc- | Glu- | Su- | Galac- | Raffi- | Stach- | Total | Total | Pro- | |
|---|---|---|---|---|---|---|---|---|---|---|---|---|---|
| Exp | Sample | tol | tol | tose | cose | crose | tinol | nose | yose | Rafs | Carbs | tein | Oil |
| Soil42 | Trans. Avg. | 0.00 | 0.01 | 0.06 | 0.03 | 6.73 | 0.09 | 0.60 | 0.19 | 0.79 | 7.71 | 37.7 | 23.1 |
| Soil42 | Null Avg. | 0.00 | 0.02 | 0.04 | 0.04 | 6.42 | 0.02 | 0.98 | 2.62 | 3.59 | 10.14 | 37.3 | 20.2 |
| | % change | | −38 | 35 | −24 | 5 | 277 | −39 | −93 | −78 | −24 | 1 | 14 |
| Soil42 | Trans. Avg. | 0.00 | 0.02 | 0.05 | 0.05 | 8.31 | 0.05 | 0.87 | 0.40 | 1.27 | 9.74 | 36.3 | 22.3 |
| Soil42 | Null Avg. | 0.00 | 0.02 | 0.04 | 0.03 | 6.39 | 0.04 | 0.92 | 2.97 | 3.89 | 10.41 | 35.0 | 20.7 |
| | % change | | −13 | 32 | 45 | 30 | 43 | −5 | −87 | −67 | −6 | 4 | 8 |
| Soil43 | Trans. Avg. | 0.02 | 0.02 | 0.14 | 0.18 | 7.34 | 0.03 | 0.74 | 0.80 | 1.54 | 9.28 | 35.0 | 22.2 |
| Soil43 | Null Avg. | 0.02 | 0.02 | 0.13 | 0.14 | 5.94 | 0.03 | 0.91 | 2.90 | 3.80 | 10.10 | 35.5 | 19.1 |
| | % change | 10 | 6 | 3 | 27 | 24 | −20 | −18 | −72 | −60 | −8 | −2 | 16 |

¹ For experiment Soil42 (AFS8720.8.6) and T0 plants AFS 8720.8.6.3 results are shown in first three numerical rows or in numerical rows 4-6 for Soil42 (AFS 8720.12.3) and T0 plants AFS 8720.12.3.1. Results for Soil43 are from event AFS 8738.11.2 and T0 plants AFS 8738.11.2.4.

In Table 13, the average oil content percent increase for transgenic T1 Soil42 seed ranges from 8 to 14% over null. Protein increase ranges from 1 to 4%. Sucrose average percent increase ranges from 5 to 30%. Total raffinosaccharide average percent decrease ranges from 67 to 78%. Total carbohydrate average percent decrease ranges from 6 to 24%.

In Table 13, the average oil content percent increase for transgenic T1 Soil43 seed is 16% over null. Protein is slightly decreased by 2%. Sucrose average percent increase ranges is 24%. Total raffinosaccharide average percent decrease is 60%. Total carbohydrate average percent decrease is 8%.

A soybean meal can be generated by one skilled in the art and the resulting protein and soluble carbohydrate compositions can be calculated for the resulting soybean meal using the composition obtained for the seed as described above. Given the average oil, protein and total soluble carbohydrate compositions shown in Table 13, the resulting average protein and soluble carbohydrate compositions can be calculated for a soybean meal, as described above, and these are shown in Table 14.

TABLE 14
Soluble carbohydrate and protein of soybean meal generated from null and transgenic Soil42 event and Soil43 events.

| | T0 | | Pini- | Sorbi- | Fruc- | Glu- | Su- | Galac- | Raffi- | Stach- | Total | Total | Pro- |
|---|---|---|---|---|---|---|---|---|---|---|---|---|---|
| Exp | Plant | Sample | tol | tol | tose | cose | crose | tinol | nose | yose | Rafs | Carbs | tein |
| Soil42 | 8720.8.6.3 | Trans. Avg. | 0.0 | 0.0 | 0.1 | 0.0 | 8.8 | 0.1 | 0.8 | 0.2 | 1.0 | 10.0 | 49.0 |
| Soil42 | 8720.8.6.3 | Null Avg. | 0.0 | 0.0 | 0.1 | 0.1 | 8.0 | 0.0 | 1.2 | 3.3 | 4.5 | 12.7 | 46.7 |
| | | Change | | −48 | 56 | −22 | 9 | 367 | −36 | −92 | −77 | −21 | 5 |
| Soil42 | 8720.12.3.1 | Trans. Avg. | 0.0 | 0.0 | 0.1 | 0.1 | 10.7 | 0.1 | 1.1 | 0.5 | 1.6 | 12.5 | 46.7 |
| Soil42 | 8720.12.3.1 | Null Avg. | 0.0 | 0.0 | 0.1 | 0.0 | 8.1 | 0.1 | 1.2 | 3.7 | 4.9 | 13.1 | 44.1 |
| | | % Change | | 2 | 28 | 70 | 33 | 28 | −3 | −86 | −67 | −5 | 6 |
| Soil43 | 8738.11.2.4 | Trans. Avg. | 0.0 | 0.0 | 0.2 | 0.2 | 9.4 | 0.0 | 1.0 | 1.0 | 2.0 | 11.9 | 45.0 |
| Soil43 | 8738.11.2.4 | Null Avg. | 0.0 | 0.0 | 0.2 | 0.2 | 7.3 | 0.0 | 1.1 | 3.6 | 4.7 | 12.5 | 43.9 |
| | | % Change | | 4 | 12 | 34 | 28 | 4 | −15 | −71 | −58 | −4 | 3 |

In Table 14, the average protein content percent increase for transgenic Soil42 soybean meal ranges from 5 to 6% over null. Sucrose average percent increase ranges from 9 to 33%. Total raffinosaccharide average percent decrease ranges from 67 to 77%. Total carbohydrate average percent decrease ranges from 5 to 21%.

In Table 14, the average protein content percent increase for transgenic Soil43 soybean meal is 3% over null. Sucrose average percent increase is 28%. Total raffinosaccharide average percent decrease is 71%. Total carbohydrate average percent decrease is 58%.

### Example 9

### Genetic Stacking of High Oleic and High Oil/High Protein Traits in Soybean

Soybean plants homozygous for the transgene of high oleic soybean event DP-305423-1 described in PCT Int. Appl. WO 2008054747 A2 which is fully incorporated by reference, was crossed with soybean plants homozygous for the transgene insertion of event AFS 4818.1.2 described in U.S. Pat. No. 8,143,476, issued Mar. 27, 2014 which is fully incorporated by reference. Briefly, emasculated flowers of AFS 4818.1.2 (comprising YL DGAT 1 and YL DGAT2) were fertilized with pollen of plants homozygous for the transgene of soybean event DP-305423-1. The resulting F1 seeds were planted and F1 plants were allowed to self-fertilize. After additional rounds of selfing of F2 and F3 plants F4 plant lines were identified that were homozygous for the HO and YL_DGAT transgenes. F5 seeds of these double homozygous plant lines were uniformly high oleic (oleic acid content >75%), uniformly reduced palmitic acid content (<5% of total fatty acids, when combined with the DP-305423-1 HO genetic background) and also exhibited the increased oil content (increase of oil seed oil content of ≧10% compared to seeds of null segregant lines gown alongside the F5 plants) associated with the YL_DGAT transgene. F5 seeds of the DP-305423-1 AFS 4818.1.2 cross were planted in double 15 ft yield trial rows comprised of 250 seeds. These rows were planted alongside T9 seed of DP-305423-1 and untransformed soybeans of the Jack genotype planted in identical fashion. At the end of the 2010 growing season composition of mature seed was analyzed by non-destructive bulk near infrared transmission spectroscopy.

### NIT Measurements, Data Analysis, and Model Development

NIR Spectra, from 850-1050 nm (2-nm step; 30-mm path length), for 400-500 g bulk samples of intact soybeans were acquired in transmission mode using a Foss Tecator AB model 1241 grain analyzer (Hoganas, Sweden) fitted with a standard instrument hopper and sample transport mechanism. Each batch was analyzed in duplicate using 10 subsample scans, which were saved as the average.

All data analysis was performed using the InfraSoft International (ISI) chemometrics software WinISI II v.1.50e (NIRSystems Inc., Silver Spring, Md., USA). Pre-treatment of the raw NIR (log 1/T) spectral data included multiplicative scatter correction and first derivative transformation over a 4-point (8-nm) gap using a 4-point smoothing function. Predictions of oil and protein content (corrected to a 13% moisture basis) were based on calibration models developed by USDA-FGIS\GIPSA. Calibration models for oleic and linolenic acid were proprietary and were developed in-house using Partial Least-Squares (PLS) regression (Williams and Norris, 1987) utilizing the transformed spectrum captured from material presenting a wide compositional diversity for these two components. The reference chemistry used for the calibrations was developed by gas chromatographic analysis of fatty acid methyl esters of oil extracts derived from the bean samples, after spectral capture. All calibration development work was performed using standardized PLS algorithms within the Win ISI II v.1.50e software. The optimum number of PLS factors was defined as that number of factors beyond which no further improvement in the Standard Error of Cross-Validation (SECV) was observed. Calculation of the SECV was handled automatically by the WinISI software. The SECV was obtained by sequentially removing subsets of samples from the calibration set, re-deriving the model and predicting the removed samples in an iterative manner. Six separate cross-validation tests provided the most reliable estimate of calibration accuracy obtainable from the sample set in question. The coefficient of determination (R²), was used to describe the correlation between reference (observed) and NIR-predicted values for the calibration set. The Relative Predictive Determinant (RPD), defined as the ratio of the SD of the reference values to the SECV, was used as a normalized indicator for comparing NIR models where values >2.0 are generally recognized as sufficient for quantitative measurement (Chang et al., 2001).

Fatty acid composition was also determined by GC analysis as described in Example 6. Table 15 demonstrates that crossing of HO and YL_DGAT soybean events allows one to effectively combine seed compositional traits comprised of increased oleic acid (at the expense of saturated and polyunsaturated seed fatty acids) and increased oil and protein content.

In Table 15, percent oil, percent protein and percent oil+protein values, as determined by NIT, are expressed as percent of seed weight. The percent fatty acids, as determined by GC-FAME analysis of oil, are expressed as weight percent of total fatty acids.

Additionally, the percent increase or decrease (percent change) for the average fatty acid composition, protein, oil or protein+oil for the F6 (Jack, DP-305423-1×Jack AFS 4818.1.2) seed compared to Jack is also shown in Table 15 where the percent is calculated in the following way; [(transgenic value-null value)/null value×100%].

TABLE 15
Seed composition of unmodified soybeans, soybeans of high oleic event DP-
305423-1, and F6 seed of a cross between DP-305423-1 and AFS 4818.1.2

| | Row | % | % | % oil + | % | % | % | % | % |
|---|---|---|---|---|---|---|---|---|---|
| Genotype | number | oil | protein | protein | palmitic | stearic | oleic | linoleic | linolenic |
| Jack | 1 | 17.7 | 35.2 | 52.9 | 9.7 | 4 | 19.4 | 58.9 | 8 |
| Jack | 2 | 17.8 | 35.5 | 53.2 | 9.7 | 4 | 21.7 | 56.9 | 7.7 |
| Jack | 3 | 17.5 | 36 | 53.5 | 9.8 | 4 | 21.1 | 57.5 | 7.7 |
| Jack | 4 | 17.6 | 35.9 | 53.5 | 9.6 | 4.1 | 20.7 | 58 | 7.6 |
| Jack | 5 | 18 | 35.4 | 53.4 | 10 | 3.9 | 20.4 | 58 | 7.8 |
| Jack | 6 | 17.6 | 35.3 | 52.9 | 9.7 | 4 | 20.6 | 57.7 | 8 |
| Jack | 7 | 17.8 | 35.2 | 53 | 9.8 | 4 | 20 | 58.4 | 7.8 |
| Jack | 8 | 18.6 | 34.8 | 53.3 | 9.7 | 4.6 | 22.1 | 56.3 | 7.4 |
| Jack | 9 | 17.7 | 35.5 | 53.2 | 9.5 | 5 | 25.1 | 53.9 | 6.4 |
| Jack | 10 | 17.7 | 36.1 | 53.8 | 9.9 | 4 | 19 | 59 | 8.1 |
| Jack | 11 | 18.3 | 34.5 | 52.8 | 9.7 | 3.7 | 21.8 | 57.8 | 7.1 |
| Jack | 12 | 18.6 | 34.6 | 53.2 | 10 | 3.6 | 19.5 | 59.7 | 7.2 |
| Jack | 13 | 18.5 | 34.4 | 52.9 | 9.6 | 3.5 | 18.8 | 60.9 | 7.4 |
| Jack | 14 | 18.5 | 35 | 53.6 | 9.9 | 4.1 | 20.8 | 57.8 | 7.5 |
| Jack | 15 | 18.6 | 34.4 | 53 | 9.8 | 3.9 | 20.8 | 57.9 | 7.6 |
| Jack | 16 | 18.5 | 34.3 | 52.8 | 9.6 | 4 | 19.7 | 59.1 | 7.7 |
| Jack | 17 | 18.1 | 34 | 52.2 | 9.8 | 4.8 | 24.5 | 54.2 | 6.6 |
| Jack | 18 | 18.7 | 33.7 | 52.4 | 9.8 | 4.5 | 24.2 | 54.6 | 6.9 |
| Jack | 19 | 17.9 | 35.8 | 53.7 | 9.7 | 4.2 | 21 | 57.6 | 7.5 |
| Jack | | 18.1 | 35 | 53.1 | 9.7 | 4.1 | 21.1 | 57.6 | 7.5 |
| (average) | | | | | | | | | |
| Jack, DP- | 1 | 17.1 | 36.9 | 54 | 6.1 | 3.5 | 85.6 | 1.8 | 3.2 |
| 305423-1, T10 | | | | | | | | | |
| Jack, DP- | 2 | 17 | 36.9 | 53.9 | 6.4 | 3.7 | 84.5 | 1.6 | 3.8 |
| 305423-1, T10 | | | | | | | | | |
| Jack, DP- | 3 | 17.1 | 37.1 | 54.2 | 6 | 4.1 | 83.5 | 2.3 | 4.2 |
| 305423-1, T10 | | | | | | | | | |
| Jack, DP- | 4 | 18 | 35.1 | 53 | 6.1 | 4.5 | 83.9 | 2 | 3.5 |
| 305423-1, T10 | | | | | | | | | |
| Jack, DP- | 5 | 17.3 | 37.2 | 54.5 | 6.2 | 3.9 | 84.6 | 1.8 | 3.6 |
| 305423-1, T10 | | | | | | | | | |
| Jack, DP- | 6 | 18.2 | 35.4 | 53.6 | 6 | 4.5 | 84.4 | 1.6 | 3.4 |
| 305423-1, T10 | | | | | | | | | |
| Jack, DP- | 7 | 17.2 | 37 | 54.2 | 6.3 | 3.7 | 84.8 | 1.6 | 3.7 |
| 305423-1, T10 | | | | | | | | | |
| Jack, DP- | | 17.4 | 36.5 | 53.9 | 6.1 | 4 | 84.4 | 1.8 | 3.6 |
| 305423-1, T10 | | | | | | | | | |
| (average) | | | | | | | | | |
| Jack, DP- | 1 | 20.4 | 38.9 | 59.3 | 4.6 | 5.6 | 82.8 | 3.6 | 3.4 |
| 305423-1 × | | | | | | | | | |
| Jack AFS | | | | | | | | | |
| 4818.1.2, F6 | | | | | | | | | |
| Jack, DP- | 2 | 21.4 | 36.9 | 58.2 | 4.1 | 5.1 | 85 | 2.9 | 2.9 |
| 305423-1 × | | | | | | | | | |
| Jack AFS | | | | | | | | | |
| 4818.1.2, F6 | | | | | | | | | |
| Jack, DP- | 3 | 21.7 | 37.1 | 58.8 | 3.8 | 5.1 | 85.7 | 2.5 | 2.9 |
| 305423-1 × | | | | | | | | | |
| Jack AFS | | | | | | | | | |
| 4818.1.2, F6 | | | | | | | | | |
| Jack, DP- | | 21.1 | 37.6 | 58.8 | 4.1 | 5.3 | 84.5 | 3 | 3 |
| 305423-1 × | | | | | | | | | |
| Jack AFS | | | | | | | | | |
| 4818.1.2, F6 | | | | | | | | | |
| (average) | | | | | | | | | |
| % Change | | 17 | 7 | 11 | −58 | 29 | 300 | −95 | −60 |

In Table 15, the average oil content of F6 (Jack, DP-305423-1×Jack AFS 4818.1.2) seed increases by 17% over Jack. The average protein content increases by 7% and the average oil+protein increases by 11%. Average palmitic acid decreases by 58%. Average stearic acid increases by 29%. Average oleic acid increases by 300%. Average linoleic acid decreases by 95%. Average alpha-linolenic acid decreases by 60%. Total saturated fatty acids percent decrease ranges from 0 to 11%.

A soybean meal can be generated by one skilled in the art and the resulting protein content can be calculated for the resulting soybean meal using the composition obtained for the seed as described above. Given the average oil and protein shown in Table 15, the resulting average protein content can be calculated for a soybean meal, as described above, and this is shown in Table 16.

TABLE 16
Protein content soybean meal generated from unmodified
soybeans, soybeans of high oleic event DP-305423-1, and
F6 seed of a cross between DP-305423-1 and AFS 4818.1.2

| Genotype | % protein |
|---|---|
| Jack (average) | 42.7 |
| Jack, DP-305423-1, T10 (average) | 44.2 |
| Jack, DP-305423-1 × Jack AFS 4818.1.2, | 47.7 |
| F6 (average) | |
| % Change | 12% |

In Table 16, the average protein content of F6 (Jack, DP-305423-1×Jack AFS 4818.1.2) soybean meal increases by 12% over Jack.

### Example 10

### Seed and Soybean Meal Compositional Change Summary for Genetic Stacking for Fatty Acid Composition, HiOil and HiProtein Traits in Soy

In Table 17, a summary of the ranges for the percent increase or decrease (percent change) for the average percent fatty acid, protein, oil and total soluble carbohydrate of transgenic seed compared to null seed is shown. Similarly, the ranges for the specific soluble carbohydrates sucrose and the total raffinosaccharides are also shown in Table 17.

TABLE 17
Range of percent changes in seed composition comparing transgenic seed to corresponding null
seed for events having altered fatty acid composition, HiOil and HiProtein Traits in Soy

| | | Fatty Acid Composition | | | | | | Seed Composition | | | | |
|---|---|---|---|---|---|---|---|---|---|---|---|---|
| | From | | | | | | Total | | | Total | Total | Total |
| Experiment | Tables- | 16:0 | 18:0 | 18:1 | 18:2 | 18:3 | Sats | Oil | Prot | Sol Carb | Rafs | Suc |
| Soil19 | 4, 5a | −7 to | 12 to | 23 to | −9 to | −73 to | 0 to | 7 to | 1 to | −24 to | −83 to | 34 to |
| | 6a | −21 | 46 | 83 | 1 | −82 | −11 | 21 | 3 | −30 | −85 | 35 |
| Soil42 | 6a 11 | −71 to | −21 to | 370 to | −87 to | −58 to | −58 to | 5 to | 1 to | −6 to | −67 to | 5 to |
| | | −74 | −24 | 414 | −91 | −66 | −61 | 14 | 4 | −24 | −78 | 30 |
| Soil43 | 11 13 | −78 | −23 | 359 | −92 | −68 | −64 | 7 to | −2 | −8 | −60 | 24 |
| | | | | | | | | 16 | | | | |
| DP-305423-1 and | 15 | −58 | 29 | 300 | −95 | −60 | | 17 | 7 | | | |
| AFS 4818.1.2 | | | | | | | | | | | | |

In Table 18, a summary of the ranges for the percent increase or decrease (percent change) for the average percent fatty acid, protein, oil and total soluble carbohydrate of transgenic soybean meal compared to null soybean meal is shown. Similarly, the ranges for the specific soluble carbohydrates sucrose and the total raffinosaccharides are also shown in Table 18.

TABLE 18
Range of percent changes in soybean meal composition
comparing transgenic soybean meal to corresponding
null soybean meal for events having altered fatty
acid composition, HiOil and HiProtein traits in Soy

| | From | | Total Sol | Total | Total |
|---|---|---|---|---|---|
| Exp | Tables | Protein | Carb | Rafs | Suc |
| Soil19 | 5b | 7 to 8 | −21 to −25 | −81 to −85 | 41 to 43 |
| | 6b | | | | |
| Soil42 | 14 | 5 to 6 | −5 to −21 | −67 to −77 | 9 to 33 |
| Soil43 | 14 | 3 | −4 | −58 | 28 |
| DP-305423-1 | 16 | 12 | | | |
| and AFS | | | | | |
| 4818.1.2 | | | | | |

### Example 11

### Identification and Cloning of the Soy Sucrose Synthase Promoter

The Arabidopsis Sucrose Synthase 2 gene has been described previously (PCT Publication No. WO 2010/114989) and the nucleotide and amino acid sequences are set forth in SEQ ID NO: 39 and SEQ ID NO: 40, respectively. A soybean homolog of the Arabidopsis Sucrose Synthase 2 gene was identified by conducting BLAST (Basic Local Alignment Search Tool; Altschul et al., J. Mol. Biol. 215:403-410 (1993)) searches for similarity to sequences contained in the Soybean Genome Project, DoE Joint Genome Institute “Glyma1.01” gene set. Specifically, the Arabidopsis Sucrose Synthase 2 amino acid sequence (SEQ ID NO: 40) was used with the TBLASTN algorithm provided by National Center for Biotechnology Information (NCBI) with default parameters except the Filter Option was set to OFF.

The soybean homolog to the Arabidopsis Sucrose Synthase 2 gene identified corresponded to Glyma13g17420 and the predicted genomic, cDNA, CDS and corresponding amino acid sequences from Glyma are set forth in SEQ IDs NO: 41, 42, 43, and 44, respectively.

Soybean cDNA libraries from developing soybean (e.g. cDNA library sdp3c) were prepared, clones sequenced and sequence was analyzed as described in U.S. Pat. No. 7,157,621 (the contents of which are herein incorporated by reference). A similar TBLASTN search against sequences from these soybean cDNA libraries identified a cDNA (EST sdp3c.pk014.n18) with a 5′ end that differed from that predicted in the Glyma13g17420 cDNA sequence (SEQ ID NO: 42) in that the intron was splice differently. The sequence for the 5′ end of EST sdp3c.pk014.n18 that was sequenced is set forth in SEQ ID NO: 45. The CDS from sdp3c.pk014.n18 appears to be the same as that for Glyma13g17420 (SEQ ID NO: 43). The soybean homolog to the Arabidopsis sucrose synthase 2 gene set forth in SEQ ID NO: 43 was named GmSus.

A region of genomic DNA upstream of the start codon of GmSus (SEQ ID NO: 43) was identified from the Glyma database by conducting BLAST searches as a promoter region and the sequence is set forth in SEQ ID NO: 46. FIG. 1 shows a schematic of the GmSus promoter region.

The identified GmSus promoter region encodes the 5′ UTR from the cDNA transcript (bp 2101 to 3191 from SEQ ID NO: 46) as well as an intron (bp 2134 to 3168 from SEQ ID NO: 46). The 5′ UTR region and intron was included as part of the promoter region as it contained an AW box (AW2 in FIG. 1) from by 2662 to 2675 of SEQ ID NO: 46 within the intron. Another AW box (AW1 in FIG. 1) occurs from by 616 to by 629 of SEQ ID NO: 46. AW boxes consist of the nucleotide sequence [CnTnG](n)7[CG] (SEQ ID NO:47), where n is any nucleotide, and AW boxes are binding sites for transcription factors such as wri1 in Arabidopsis (Maeo, K et al. (2009) Plant Journal 60(3): 476-487).

Genomic DNA was isolated from leaves of approximately 4 week old soy 93B86 plants using the DNEASY® Plant Mini Kit (Qiagen, Valencia, Calif.) and following the manufacture's protocol. The GmSus promoter region (SEQ ID NO:46) was PCR-amplified from 93B86 genomic DNA using oligonucleotides GmSuSyProm-5 (SEQ ID NO:48) and GmSuSyProm-5 (SEQ ID NO:49) with the PHUSION™ High-Fidelity DNA Polymerase (Cat. No. F553S, Finnzymes Oy, Finland), following the manufacturer's protocol. The resulting DNA fragment was cloned into the pCR®-BLUNT® cloning vector using the ZERO BLUNT® PCR Cloning Kit (Invitrogen Corporation), following the manufacturer's protocol, to produce pLF284 (SEQ ID NO:50).

The EcoRI fragment of pLF284 (SEQ ID NO: 50), containing the GmSus promoter region (called GmSusPro), was cloned into the EcoRI site of pNEB193 (New England BioLabs, Beverly, Mass.) to produce pKR1963 (SEQ ID NO: 51).

Plasmid pKR1543, which was previously described in PCT Publication No. WO 2011/079005 (published on Jun. 30, 2011, the contents of which are herein incorporated by reference), was digested with NotI/XbaI and the fragment containing the Leg terminator, previously described in PCT Publication No. WO 2004/071467 (published on Aug. 26, 2004, the contents of which are herein incorporated by reference) was cloned into the NotI/XbaI fragment of pKR1963 (SEQ ID NO: 51), containing the GmSusPro, to produce pKR1964 (SEQ ID NO: 52).

The BsiWI fragment of pKR1964 (SEQ ID NO: 52), containing the GmSusPro, was cloned into the BsiWI site of pKR325, previously described in PCT Publication No. WO 2004/071467, to produce pKR1965 (SEQ ID NO: 53). Plasmid pKR1965 contains a NotI site flanked by the GmSusPro and the Leg terminator as well as the hygromycin B phosphotransferase gene [Gritz, L. and Davies, J. (1983) Gene 25:179-188], flanked by the T7 promoter and transcription terminator, a bacterial origin of replication (ori) for selection and replication in E. coli and the hygromycin B phosphotransferase gene, flanked by the 35S promoter [Odell et al., (1985) Nature 313:810-812] and NOS 3′ transcription terminator [Depicker et al., (1982) J. Mol. Appl. Genet. 1:561:570] (35S/hpt/NOS3′ cassette) for selection in soybean. In this way, polynucleotides (e.g., protein-coding regions) flanked by NotI sites can be cloned into the NotI site of pKR1965 (SEQ ID NO: 53) and expressed in soy.

### Example 12

### Cloning LED and ODP1 Homologs from Soybean

GmLec1 from cDNA:

Soybean cDNA library se2, derived from developing soybean seeds (Glycine max L.) harvested at 13 days after flowering (DAF) was prepared, cDNA clones were sequenced and the sequence was analyzed as described in U.S. Pat. No. 7,157,621.

A cDNA clone (se2.11d12) was identified from cDNA library se2 with homology to transcription factor LEAFY COTYLEDON1 (Led) (Lotan, T. et al. (1998) Cell 93(7): 1195-1205).

The cDNA clone was fully sequenced by methods described in U.S. Pat. No. 7,157,621 and its sequence is set forth in SEQ ID NO: 54. This clone appears to have 2 separate cDNA clones inserted into it but the sequence from 38-718 by is 100% identical to the coding sequence of lec1b (NCBI Accession # EU088289.1 GI:158525282) and to the CDS of Glyma17g00950 based on a blast comparison. The coding sequence from clone se2.11d12, which corresponds to that of Glyma17g00950, is shown in SEQ ID NO:55 and the encoded amino acid sequence is shown in SEQ ID NO:56.

A separate cDNA clone (se1.pk0042.d8) identified from cDNA library se1, derived from developing soybean seeds (Glycine max L.) harvested at 6-10 DAF and described in U.S. Pat. No. 7,157,621, also contained a led homolog as determined by blast analysis. The full insert sequence of se1.pk0042.d8 is shown in SEQ ID NO:57. The sequence from cDNA clone se1.pk0042.d8 is 99% identical to the coding sequence of lec1a (NCBI Accession # EU088288.1 GI:158525280) and 100% identical to the CDS of Glyma07g39820 based on a blast comparison. The coding sequence from clone se1.pk0042.d8 appears to be 2 nt short of the ATG but is shown in SEQ ID NO: 58 with the correct start as compared to Glyma07g39820. The corresponding encoded amino acid sequence is shown in SEQ ID NO: 59.

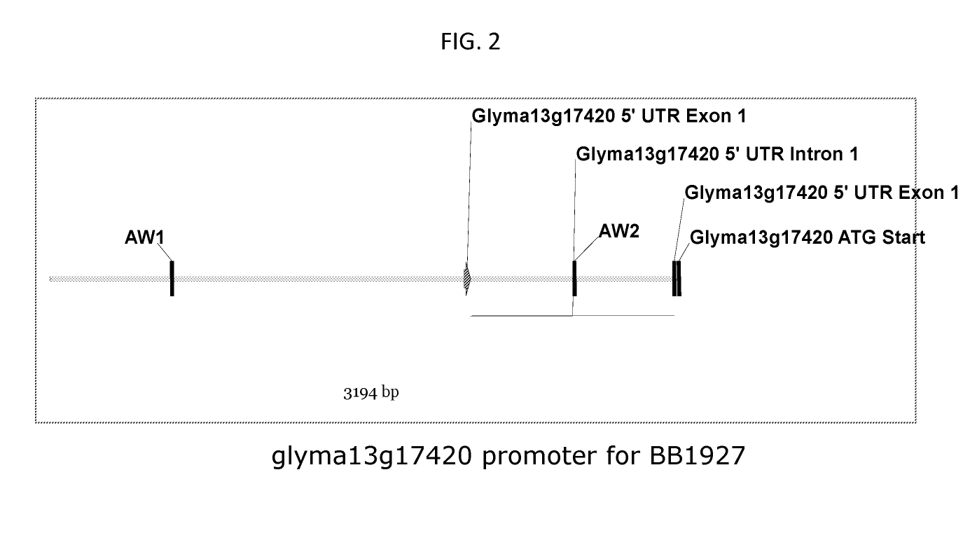

DNA was also prepared from an aliquot of cDNA library se2 using the QIAprep® Spin Miniprep Kit (Qiagen Inc., Valencia, Calif.) following the manufacturer's protocol. The DNA from the cDNA library was used as template in a PCR reaction using oligonucleotides SA275 (SEQ ID NO: 60) and SA276 (SEQ ID NO: 61), using the “Platinum”-brand Taq DNA polymerase (Life Technologies), following the manufacturer's protocol. The PCR fragment was cloned using the pCR®8/GW/TOPO® TA Cloning Kit (Invitrogen Corporation) to produce plasmid Glyma17g00950/pCR8/GW/TOPO (SEQ ID NO: 62). The CDS from the PCR product contained in Glyma17g00950/pCR8/GW/TOPO (SEQ ID NO: 62), named GmLec1, is set forth in SEQ ID NO: 63 and the corresponding amino acid sequence of GmLec1 is set forth in SEQ ID NO: 64. It should be noted that both the CDS and amino acid sequence of GmLec1 are different than those corresponding to either Glyma17g00950 or Glyma07g39820. An alignment comparing the amino acid sequences of Glyma17g00950 (SEQ ID NO: 56), Glyma07g39820 (SEQ ID NO: 59) and GmLec1 (SEQ ID NO: 64) is shown in FIG. 2.

GmLec1 gene was PCR-amplified from Glyma17g00950/pCR8/GW/TOPO (SEQ ID NO: 62) using oligonucleotides Gmlec-5 (SEQ ID NO:65) and Gmlec-3 (SEQ ID NO:66) with the PHUSION™ High-Fidelity DNA Polymerase (Cat. No. F553S, Finnzymes Oy, Finland), following the manufacturer's protocol. The PCR fragment was cloned into the pCR®-BLUNT® cloning vector using the ZERO BLUNT® PCR Cloning Kit (Invitrogen Corporation), following the manufacturer's protocol, to produce pLF275 (SEQ ID NO: 67).

### NotI Fragment Containing GmODP1:

The soybean ODP (GmODP1) is described in U.S. Pat. No. 7,157,621. The cloning of GmODP1 with flanking NotI sites into plasmid KS334 was previously described in PCT Publication No. WO 2010/114989 (published on Oct. 7, 2010, the contents of which are herein incorporated by reference). It should be noted that there is a typo in the map of KS334 (SEQ ID NO: 14 in WO2010/114989) and that there should be an additional 3 nucleotides (TGA) at position 1237 to form a stop codon and end the CDS in KS334. The CDS and amino acid sequence of GmODP1 from WO2010/114989 are set forth here in SEQ ID NO: 68 and SEQ ID NO: 69, respectively.

### Example 13

### Expressing GmLec1 and GmODP1 in

### Soybean Seed Under Control of the GmSus Promoter

Artificial microRNAs Silencing Fad2 Genes as Reporter for Transgenic Events:

The fatty acid desaturase 2-1 (Fad2-1) or 2-2 (fad2-2) gene families (Heppard, E P, et al. (1996) Plant Physiology, 110(1): 311-319), also known as delta-12 desaturase or omega-6 desaturase (US Patent Numbers U.S. Pat. No. 6,872,872B1, U.S. Pat. No. 6,919,466B2 and U.S. Pat. No. 7,105,721B2), convert oleic acid into linoleic acid. Effective silencing of the fad2-1 and fad2-2 gene families seed-specifically in soy results in seed oil having an increased oleic acid content which can be detected using methods known to one skilled in the art such as those described herein. This increased oleic acid content can be used as a reporter to identify transgenic seed in segregating seed populations from null seed.

The design and synthesis of artificial microRNAs (amiRNAs), and the respective STAR sequences that pair with amiRNAs, for silencing the soy fad2-1 and fad2-2 genes was previously described in US20090155910A1 (WO 2009/079532) (the contents of which are incorporated by reference) and the sequences are described in Table 19.

TABLE 19
amiRNA and Star sequences for soy fad2-1 and fad2-2

| Gene | | SEQ | | SEQ |
|---|---|---|---|---|
| Family | amiRNA | ID NO | STAR Sequence | ID NO |
| GmFad2-1 | GM-MFAD2-1B | 84 | 396b-GM-MFAD2-1B | 70 |
| GmFad2-2 | GM-MFAD2-2 | 85 | 159-GM-MFAD2-2 | 71 |

The identification of the genomic miRNA precursor sequences 159 and 396b was described previously in US20090155910A1 (WO 2009/079532) and their sequences are set forth in SEQ ID NO: 72 and SEQ ID NO: 73, respectively.

Genomic miRNA precursor sequences 159 (SEQ ID NO: 72) and 396b (SEQ ID NO: 73) were converted to amiRNA precursors 396b-fad2-1 b and 159-fad2-2 using overlapping PCR as previously described in US20090155910A1 (WO 2009/079532).

amiRNA precursor 159-fad2-2 was cloned downstream of 396b-fad2-1 b to produce the amiRNA precursor 396b-fad2-1b/159-fad2-2 (SEQ ID NO: 74).

The amiRNA precursor 396b-fad2-1 b/159-fad2-2 (SEQ ID NO: 74) is 1577 nt in length and is substantially similar to the deoxyribonucleotide sequence set forth in SEQ ID NO: 73 (from nt 1 to 574 of 396b-fad2-1b/159-fad2-2) wherein nucleotides 196 to 216 of SEQ ID NO: 73 are replaced by GM-MFAD2-1B amiRNA (SEQ ID NO: 84) and wherein nucleotides 262 to 282 of SEQ ID NO: 73 are replaced by 396b-GM-MFAD2-1B Star Sequence (SEQ ID NO: 70). The amiRNA precursor 396b-fad2-1b/159-fad2-2 (SEQ ID NO: 74) is also, substantially similar to the deoxyribonucleotide sequence set forth in SEQ ID NO: 72 (from nt 620 to 1577 of 396b-fad2-1b/159-fad2-2) wherein nucleotides 276 to 296 of SEQ ID NO: 72 are replaced by GM-MFAD2-2 amiRNA (SEQ ID NO: 85) and wherein nucleotides 121 to 141 of SEQ ID NO: 72 are replaced by 159-GM-MFAD2-2 Star Sequence (SEQ ID NO: 71). In amiRNA precursor 396b-fad2-1b/159-fad2-2, nt 575 to 610 are derived from cloning.

Construction of Soybean Expression Vector pKR2109:

Using standard PCR and cloning methods by one skilled in the art, the following DNA elements were assembled to produce the 8095 by soybean expression vector pKR2109 (SEQ ID NO: 75) and having unique SbfI (nt 8093) and BsiWI (nt 1) restriction sites for cloning expression cassettes.

In pKR2109 (SEQ ID NO: 75), sequence 21-36 is a sequence of DNA comprising ORF stop codons in all 6 frames (ORFSTOP-A). Sequence 65-2578 is vector backbone containing the T7 promoter (sequence 1297-1394), the hygromycin phosphotransferase (hpt) gene coding region (sequence 1395-2435) and the T7 terminator (sequence 2436-2582). Sequence 2616-2632 is a sequence of DNA comprising ORF stop codons in all 6 frames (ORFSTOP-B). Sequence 2698-4006 is the constitutive soy SAMS promoter (U.S. Pat. No. 7,217,858). Sequence 4011-4058 is a FLP recombinase recognition site FRT1 (U.S. Pat. No. 8,293,533). Sequence 4068-5093 is the hygromycin phosphotransferase (hpt) gene coding region for selection in soy. Sequence 5102-5382 is the NOS 3′ transcription terminator (Depicker et al., J. Mol. Appl. Genet. 1:561-570 (1982)). Sequence 5400-6170 is the 776 by fragment of the soy annexin promoter (described in Applicants' Assignee's U.S. Pat. No. 7,129,089). Sequence 6179-7756 is the amiRNA precursor 396b-fad2-1 b/159-fad2-2 (SEQ ID NO: 74). Sequence 7773-7988 is the soy BD30 transcription terminator (described in Applicants' Assignee's U.S. Pat. No. 8,084,074). Sequence 8021-8068 is a FLP recombinase recognition site FRT87 (U.S. Pat. No. 8,293,533).

### Expressing GmLec1 and GmODP1 Soybean Under Control of the GmSus Promoter:

The NotI fragment of pLF275 (SEQ ID NO: 67, containing GmLec1 and the NotI fragment of KS334, containing GmODP1 were cloned into the NotI site of pKR1965 (SEQ ID NO: 53) to produce pKR1968 (SEQ ID NO: 76) and pKR1971 (SEQ ID NO: 77), respectively.

The SbfI fragments of pKR1968 (SEQ ID NO: 76), containing GmLec1 and pKR1971 (SEQ ID NO: 77), containing GmODP1 were cloned into the SbfI site of pKR2109 (SEQ ID NO: 75) to produce pKR2118 (SEQ ID NO: 78) and pKR2120 (SEQ ID NO: 79), respectively.

Each experiment was given a name and a summary of the experiment name, construct used and genes expressed is shown in Table 20.

TABLE 20
Summary of genes, plasmids and experiments

Gene SEQ ID NO

| Experiment | Plasmid | Gene | nt | aa |
|---|---|---|---|---|
| Oil109 | pKR2120 | GmODP1 | 80 | 81 |
| Oil110 | pKR2118 | GmLec1 | 82 | 83 |

DNA from these plasmids was prepared for particle bombardment into soybean embryogenic suspension culture and transformed exactly as described previously in PCT Publication No. WO 2008/147935. Soybean embryogenic suspension culture was initiated, grown and maintained and events were selected and matured exactly as described in PCT Publication No. WO 2008/147935. In this case, hygromycin was used for selection. Events from each of the 3 experiments were screened at the embryo stage for fatty acid profile by methods described herein and those displaying an increased oleic acid phenotype were advanced.

Embryos from selected events were dried and germinated and T0 plants were grown and maintained exactly as described in PCT Publication No. WO 2008/147935.

Approximately 36 T1 seeds from T0 plants for each event were harvested and individual T1 seed were analyzed for oil and protein content using Near Infrared Spectroscopy by methods familiar to one skilled in the art [Agelet, et al. (2012) Journal of Agricultural and Food Chemistry, 60(34): 8314-8322].

Seeds were also analyzed for fatty acid profile in order to identify transgenic and null seed. Those seed having oleic acid contents higher than approximately 30%, resulting from expression of the amiRNA precursor 396b-fad2-1b/159-fad2-2, were considered transgenic. Those with approximately less than 30% oleic acid content were considered null seed.

For each event, the average oil content of all transgenic seed and all null seed was determined. The average oil content of null seed was then subtracted from the average oil content of the transgenic seed and the difference is reported in Table 35 (Avg. Oil Delta %). The difference in average protein content between transgenic and null seed was similarly determined and is shown in Table 35 (Avg. Pro Delta %). The sum of the Avg. Oil Delta % and Avg. Pro Delta % (Avg. Proil Delta %) is also shown in Table 35. For a representative number of events of each construct at least 24 seeds were germinated in soil and germination rate was determined 10 days after planting.

In Table 21, the experiment name (Exp.), the gene being expressed (Gene) and the event name (Event) are also shown.

TABLE 21
Summary of difference in average oil and protein
contents between transgenic and null T1 seed for
soybean events expressing GmLec1 or GmODP1.

| | | | Avg. | Avg. | Avg. | Germi- |
|---|---|---|---|---|---|---|
| | | | Oil | Pro | Proil | nation |
| Exp. | Gene | Event | Delta % | Delta % | Delta % | % |
| Oil 109 | GmODP1 | 8810.5.1 | 1.9 | 2.4 | 4.3 | 99 |
| Oil 109 | GmODP1 | 8787.3.3 | 1.2 | 1.9 | 3.1 | 95 |
| Oil 109 | GmODP1 | 8787.12.2 | 0.4 | 2.4 | 2.8 | 90 |
| Oil 109 | GmODP1 | 878710.1 | 1.4 | 0.9 | 2.2 | 87 |
| Oil 109 | GmODP1 | 8787.4.1 | 0.7 | 1.4 | 2 | |
| Oil 109 | GmODP1 | 8787.8.4 | 1.1 | 0.8 | 1.9 | |
| Oil 109 | GmODP1 | 8787.10.5 | −0.2 | 1.8 | 1.7 | |
| Oil 109 | GmODP1 | 8787.7.3 | 1.3 | 0.4 | 1.7 | 79 |
| Oil 109 | GmODP1 | 8787.3.2 | 0.3 | 0.8 | 1.1 | |
| Oil 109 | GmODP1 | 8787.1.1 | −0.2 | 1 | 0.8 | 85 |
| Oil 109 | GmODP1 | 8787.6.4 | 0.2 | 0.4 | 0.7 | |
| Oil 109 | GmODP1 | 8787.12.3 | 1.7 | −1 | 0.6 | 95 |
| Oil 109 | GmODP1 | 8787.11.4 | 0 | 0.5 | 0.5 | 94 |
| Oil 109 | GmODP1 | 8787.6.3 | −1.5 | 0.5 | −1 | 83 |
| Oil 110 | GmLec1 | 8781.6.1 | 1 | 2 | 2.9 | 33 |
| Oil 110 | GmLec1 | 8781.2.2 | 0.9 | 1.8 | 2.8 | 91 |
| Oil 110 | GmLec1 | 8781.2.3 | 1.2 | 1.5 | 2.8 | 81 |
| Oil 110 | GmLec1 | 8781.10.5 | 0.9 | 1.9 | 2.8 | 81 |
| Oil 110 | GmLec1 | 8781.3.6 | 0.8 | 1.5 | 2.3 | 32 |
| Oil 110 | GmLec1 | 8781.11.2 | 0.7 | 1.3 | 2 | 69 |
| Oil 110 | GmLec1 | 8781.11.1 | 0.3 | 0.5 | 0.7 | |

Table 21 shows that average oil and protein content is increased when GmODP1 or GmLec1 is over-expressed in soybean under control of the GmSus promoter when compared to the average of null seed. Oil and protein (Proil) is increased by as high as 2.9 to 4.3 points in these events. Table 21 also shows that T1 seed germination frequency of events with significant oil and protein increase due to expression of ODP1, LEC1 and Fusca3 transcription factors can be as high as 99%, 91% and 78%, respectively.

T1 seed from events segregating as single copy (HiOleic Phenotype:Null=3:1) were planted, plants were grown exactly as for T0 plants and T2 seed were obtained. T2 seed from these events were analyzed for oleic acid and for oil and protein content by ssNIR exactly as described herein and results are shown for Oil109 in Table 22 and for Oil110 in Table 23. Classification of seed as being from a homozygous plant, heterozygous plant or null plant was determined using oleic acid, oil and protein data as well as using construct-specific quantitative PCR (qPCR) as described previously in U.S. Pat. No. 8,293,533, which issued Oct. 23, 2012 results on leaf punches from T1 plants as described herein.

For each event, the average oil content of all transgenic homozygous T2 seed and all null seed was determined. The average oil content of null seed was then subtracted from the average oil content of the homozygous T2 transgenic seed and the difference is reported in Table 22 (Avg. Oil Delta %). The difference in average protein content between T2 homozygous transgenic and null seed was similarly determined and is shown in Table 22 and Table 23 (Avg. Pro Delta %). The sum of the Avg. Oil Delta % and Avg. Pro Delta % (Avg. Proil Delta %) is also shown in

Table 22 and Table 23.

TABLE 22
Summary of difference in average oil and protein
contents between homozygous transgenic and null
T2 seed for soybean events expressing GmODP1

| | | | Avg. | Avg. | Avg. |
|---|---|---|---|---|---|
| | | | Oil | Pro | Proil |
| Exp. | Gene | Event | Delta % | Delta % | Delta % |
| Oil 109 | GmODP1 | 8787.10.1 | 1.8 | 2.8 | 4.7 |
| Oil 109 | GmODP1 | 8787.7.3 | 1.3 | 2.9 | 4.2 |
| Oil 109 | GmODP1 | 8810.5.1 | 1.5 | 1.5 | 3.0 |

Table 22 shows that average oil and protein content is increased when GmODP1 is over-expressed in soybean under control of the GmSus promoter when compared to the average of null seed. Oil and protein are increased by as high as 3.0 to 4.7 points in these single copy events.

TABLE 23
Summary of difference in average oil and protein
contents between homozygous transgenic and null
T2 seed for soybean events expressing GmLec1

| | | | Avg. | Avg. | Avg. |
|---|---|---|---|---|---|
| | | | Oil | Pro | Proil |
| Exp. | Gene | Event | Delta % | Delta % | Delta % |
| Oil 110 | GmLEC1 | 8781.11.2 | 2.6 | 1.1 | 3.7 |
| Oil 110 | GmLEC1 | 8781.3.6 | 0.8 | 1.0 | 1.8 |
| Oil 110 | GmLEC1 | 8781.7.8 | 1.3 | 2.9 | 4.1 |

Table 23. shows that average oil and protein content is increased when GmLEC1 is over-expressed in soybean under control of the GmSus promoter when compared to the average of null seed. Oil and protein are increased by as high as 1.8 to 4.1 points in these single copy events.

Compositional Analysis on Bulk T2 Seed from 011109 and 011110 Events:

T2 seed from homozygous and null plants from Oil 109 event 8787.7.3 (GmODP1) and Oil110 event 8781.7.8 (GmLEC1) were collected and 5 representative seed from each plant were crushed in a genogrinder as described so herein. Total oil content was determined by NMR and total protein content was determined using the combustion analyzer, on soy powders, exactly as described herein. Further, lipids were extracted from soy powders using heptane extraction and fatty acid composition was determined using GC-FAME analysis as described herein. Also, total non-structural soluble carbohydrate was extracted and quantified, and composition determined, using GC as described herein.

The results for fatty acid composition for T2 seed bulks from each homozygous transgenic plant, and the results for T2 seed bulks from each null plant are presented for both in Table 24. The total saturated fatty acid content (% Tot Sats) is calculated as the sum of the percent 16:0 and percent 18:0. The results for each plant were also averaged together and these averages are also presented in Table 24. In Table 24, fatty acid composition and average fatty acid composition for lipids from seeds from all homozygous transgenic or null plants are reported as a percent of total fatty acid.

The change in fatty acid composition (% FA transgenic−% FA null) and the percent increase or decrease (percent change) for the average of any particular fatty acid as is also shown in Table 24 where the percent is calculated in the following way; [(transgenic value-null value)/null value×100%].

Additionally, the results for oil content (% Oil; calculated as percent of soybean powder) and protein content (% Prot; calculated as percent of soybean powder) for null and transgenic seed are also shown in Table 24, as are the values for the sum of protein and oil content (% Proil; calculated as sum of % Oil and % Protein). The average percent Oil, percent Prot and percent Proil is also summarized in Table 24 as is the change and percent change, calculated as described for fatty acid composition.

TABLE 24
Fatty acid composition of lipids and percent Oil, percent Prot and percent Proil from bulk T2
seed powders from null and homozygous transgenic Oil109 (GmODP1) or Oil10 (GmLEC1) events.

| | | T2 | | % | % | | | | | | | % Tot |
|---|---|---|---|---|---|---|---|---|---|---|---|---|
| Exp | Event/Plant | Seed Pack | Type | Oil | Protein | Proil | %16:0 | %18:0 | %18:1 | %18:2 | %18:3 | Sats |
| Oil109 | 8787.7.3.4 | 12SN37-2200 | Null | 18.7 | 37.3 | 55.9 | 10.8 | 3.8 | 25.1 | 53.7 | 6.6 | 14.6 |
| Oil109 | 8787.7.3.4 | 12SN37-2305 | Null | 20.4 | 35.0 | 55.3 | 10.3 | 4.0 | 23.5 | 55.7 | 6.5 | 14.3 |
| Oil109 | 8787.7.3.4 | 12SN37-2341 | Null | 18.9 | 37.2 | 56.1 | 10.5 | 4.2 | 28.9 | 50.8 | 5.6 | 14.7 |
| | | | Avg. | 19.3 | 36.5 | 55.8 | 10.5 | 4.0 | 25.8 | 53.4 | 6.2 | 14.5 |
| Oil109 | 8787.7.3.4 | 12SN37-2269 | Trans | 19.9 | 38.3 | 58.2 | 6.9 | 3.5 | 87.9 | 0.5 | 1.2 | 10.4 |
| Oil109 | 8787.7.3.4 | 12SN37-2377 | Trans | 20.6 | 37.7 | 58.3 | 6.7 | 3.6 | 87.8 | 0.7 | 1.2 | 10.3 |
| | | | Avg. | 20.3 | 38.0 | 58.2 | 6.8 | 3.6 | 87.8 | 0.6 | 1.2 | 10.4 |
| OIL109 | | | Change | 1.0 | 1.5 | 2.5 | −3.7 | −0.5 | 62.0 | −52.8 | −5.0 | −4.2 |
| Oil109 | | | % Change | 5 | 4 | 4 | −35 | −11 | 240 | −99 | −81 | −29 |
| Oil110 | 8781.7.8.2 | 12GR27-200 | Null | 18.1 | 34.8 | 52.9 | 10.8 | 3.7 | 17.3 | 59.6 | 8.5 | 14.6 |
| Oil110 | 8781.7.8.2 | 12GR27-202 | Null | 18.0 | 38.0 | 56.0 | 10.7 | 3.7 | 20.9 | 57.3 | 7.3 | 14.4 |
| | | | Avg. | 18.0 | 36.4 | 54.4 | 10.8 | 3.7 | 19.1 | 58.5 | 7.9 | 14.5 |
| Oil110 | 8781.7.8.2 | 12GR27-174 | Avg. | 19.1 | 37.8 | 56.9 | 8.1 | 3.6 | 83.9 | 1.6 | 2.9 | 11.7 |
| Oil110 | 8781.7.8.2 | 12GR27-176 | Avg. | 18.2 | 38.8 | 57.0 | 7.1 | 3.6 | 85.0 | 1.4 | 2.8 | 10.7 |
| Oil110 | 8781.7.8.2 | 12GR27-184 | Avg. | 18.5 | 38.2 | 56.7 | 7.4 | 3.6 | 84.0 | 1.7 | 3.3 | 11.0 |
| | | | Avg. | 18.6 | 38.3 | 56.9 | 7.5 | 3.6 | 84.3 | 1.6 | 3.0 | 11.1 |
| Oil110 | | | Change | 0.5 | 1.9 | 2.4 | −3.3 | −0.1 | 65.2 | −56.9 | −4.9 | −3.4 |
| Oil110 | | | % Change | 3 | 5 | 4 | −30 | −3 | 341 | −97 | −62 | −23 |

The results for individual, non-structural carbohydrate content for T2 seed bulks from each homozygous transgenic and null plant are presented for in Table 25. In Table 25, non-structural, soluble carbohydrate content (pinitol, sorbitol, fructose, glucose, sucrose, galactinol, raffinose, stachyose) are reported as a percent of ground soy powder. Also presented are the total rafinosaccharides (% Total Rafs; calculated as the sum of raffinose and stachyose) and the total soluble carbohydrates (% Total Carbs; calculated as the sum of all individual carbohydrates). The results for each plant were also averaged together and these averages are also presented in Table 25 as is the change in soluble carbohydrate composition and the percent increase or decrease (percent change) for the average of any particular carbohydrate calculated as described herein.

TABLE 25
Non-structural, soluble carbohydrate content of bulk T2 seed powders from
null and homozygous transgenic Oil109 (GmODP1) or Oil110 (GmLEC1) events.

| | | T2 | | Pini- | Sorbi- | Fruc- | Glu- | Su- | Galac- | Raffi- | Stachy- | % Total | % Total |
|---|---|---|---|---|---|---|---|---|---|---|---|---|---|
| Exp | Event/Plant | Seed Pack | Type | tol | tol | tose | cose | crose | tinol | nose | ose | Carbs | Rafs |
| Oil109 | 8787.7.3.4 | 12SN37-2200 | Null | 0.3 | 0.0 | 0.0 | 0.0 | 4.2 | 0.0 | 0.7 | 3.9 | 9.2 | 4.6 |
| Oil109 | 8787.7.3.4 | 12SN37-2305 | Null | 0.6 | 0.0 | 0.0 | 0.0 | 4.5 | 0.0 | 0.7 | 3.4 | 9.2 | 4.1 |
| Oil109 | 8787.7.3.4 | 2341 | Null | 0.7 | 0.0 | 0.1 | 0.0 | 3.8 | 0.0 | 0.8 | 3.3 | 8.7 | 4.1 |
| | | | Avg. | 0.5 | 0.0 | 0.0 | 0.0 | 4.2 | 0.0 | 0.7 | 3.5 | 9.0 | 4.3 |
| Oil109 | 8787.7.3.4 | 12SN37-2269 | Trans | 0.4 | 0.0 | 0.1 | 0.0 | 3.8 | 0.0 | 0.7 | 3.5 | 8.4 | 4.1 |
| Oil109 | 8787.7.3.4 | 12SN37-2377 | Trans | 0.5 | 0.0 | 0.0 | 0.0 | 4.2 | 0.0 | 0.6 | 3.6 | 9.1 | 4.3 |
| | | | Avg. | 0.5 | 0.0 | 0.1 | 0.0 | 4.0 | 0.0 | 0.6 | 3.5 | 8.7 | 4.2 |
| Oil109 | | | Change | −0.1 | 0.0 | 0.0 | 0.0 | −0.2 | 0.0 | −0.1 | 0.0 | −0.3 | −0.1 |
| Oil109 | | | % Change | −0.1 | 0.1 | 0.2 | 0.0 | 0.0 | 0.4 | −0.1 | 0.0 | 0.0 | 0.0 |
| Oil110 | 8781.7.8.2 | 12GR27-200 | Null | 0.3 | 0.0 | 0.0 | 0.0 | 6.4 | 0.0 | 1.2 | 3.5 | 11.4 | 4.7 |
| Oil110 | 8781.7.8.2 | 12GR27-202 | Null | 0.4 | 0.0 | 0.0 | 0.0 | 6.3 | 0.0 | 1.1 | 3.1 | 11.0 | 4.2 |
| | | | Avg. | 0.3 | 0.0 | 0.0 | 0.0 | 6.4 | 0.0 | 1.1 | 3.3 | 11.2 | 4.4 |
| Oil110 | 8781.7.8.2 | 12GR27-174 | Avg. | 0.2 | 0.0 | 0.0 | 0.0 | 5.1 | 0.0 | 0.8 | 2.9 | 9.0 | 3.6 |
| Oil110 | 8781.7.8.2 | 12GR27-176 | Avg. | 0.3 | 0.0 | 0.0 | 0.0 | 4.5 | 0.0 | 1.0 | 3.3 | 9.1 | 4.3 |
| Oil110 | 8781.7.8.2 | 12GR27-184 | Avg. | 0.1 | 0.0 | 0.0 | 0.0 | 4.9 | 0.0 | 0.8 | 3.4 | 9.3 | 4.2 |
| | | | | 0.2 | 0.0 | 0.0 | 0.0 | 4.8 | 0.0 | 0.9 | 3.2 | 9.2 | 4.0 |
| Oil110 | | | Change | −0.1 | 0.0 | 0.0 | 0.0 | −1.5 | 0.0 | −0.3 | −0.1 | −2.1 | −0.4 |
| Oil110 | | | % Change | −36 | −15 | 2 | −75 | −24 | 1 | −25 | −4 | −18 | −9 |

A soybean meal can be generated by one skilled in the art and the resulting protein and soluble carbohydrate compositions can be calculated for the resulting soybean meal using the composition obtained for the seed as described above. Given the average oil, protein and total soluble carbohydrate compositions shown in Table 25, the resulting average protein and soluble carbohydrate compositions can be calculated for a soybean meal, as described herein, and these are shown in Table 26.

TABLE 26
Soluble carbohydrate and protein of calculated for a soybean meal generated from null and transgenic Oil109 and Oil110 events.

| | | T2 | | % | Pini- | Sorbi- | Fruc- | Glu- | Su- | Galac- | Raffi- | Stachy- | % Total | % Total |
|---|---|---|---|---|---|---|---|---|---|---|---|---|---|---|
| Experiment | Event/Plant | Seed Pack | Type | Protein | tol | tol | tose | cose | crose | tinol | nose | ose | Carbs | Rafs |
| Oil109 | 8787.7.3.4 | 12SN37-2200 | Null | 45.8 | 0.4 | 0.0 | 0.0 | 0.0 | 5.2 | 0.0 | 0.8 | 4.8 | 11.3 | 5.7 |
| Oil109 | 8787.7.3.4 | 12SN37-2305 | Null | 43.9 | 0.7 | 0.0 | 0.1 | 0.0 | 5.6 | 0.0 | 0.9 | 4.3 | 11.6 | 5.2 |
| Oil109 | 8787.7.3.4 | 12SN37-2341 | Null | 45.8 | 0.9 | 0.0 | 0.1 | 0.0 | 4.7 | 0.0 | 0.9 | 4.1 | 10.8 | 5.0 |
| | | | Avg. | 45.2 | 0.7 | 0.0 | 0.1 | 0.0 | 5.2 | 0.0 | 0.9 | 4.4 | 11.2 | 5.3 |
| Oil109 | 8787.7.3.4 | 12SN37-2269 | Trans | 47.8 | 0.5 | 0.0 | 0.1 | 0.0 | 4.7 | 0.0 | 0.8 | 4.3 | 10.5 | 5.1 |
| Oil109 | 8787.7.3.4 | 12SN37-2377 | Trans | 47.5 | 0.7 | 0.0 | 0.1 | 0.0 | 5.3 | 0.0 | 0.8 | 4.6 | 11.4 | 5.4 |
| | | | Avg. | 47.6 | 0.6 | 0.0 | 0.1 | 0.0 | 5.0 | 0.0 | 0.8 | 4.4 | 11.0 | 5.2 |
| Oil109 | | | Change | 2.4 | −0.1 | 0.0 | 0.0 | 0.0 | −0.2 | 0.0 | −0.1 | 0.1 | −0.2 | 0.0 |
| Oil109 | | | % Change | 0.1 | −0.1 | 0.1 | 0.2 | 0.0 | 0.0 | 0.4 | −0.1 | 0.0 | 0.0 | 0.0 |
| Oil110 | 8781.7.8.2 | 12GR27-200 | Null | 42.5 | 0.3 | 0.1 | 0.0 | 0.0 | 7.8 | 0.0 | 1.4 | 4.3 | 13.9 | 5.7 |
| Oil110 | 8781.7.8.2 | 12GR27-202 | Null | 46.3 | 0.4 | 0.1 | 0.0 | 0.0 | 7.7 | 0.0 | 1.3 | 3.8 | 13.4 | 5.2 |
| | | | | 44.4 | 0.4 | 0.1 | 0.0 | 0.0 | 7.8 | 0.0 | 1.4 | 4.0 | 13.7 | 5.4 |
| Oil110 | 8781.7.8.2 | 12GR27-174 | Avg. | 46.7 | 0.3 | 0.0 | 0.0 | 0.0 | 6.3 | 0.0 | 0.9 | 3.5 | 11.1 | 4.5 |
| Oil110 | 8781.7.8.2 | 12GR27-176 | Avg. | 47.4 | 0.3 | 0.1 | 0.0 | 0.0 | 5.5 | 0.0 | 1.2 | 4.0 | 11.2 | 5.2 |
| Oil110 | 8781.7.8.2 | 12GR27-184 | Avg. | 46.9 | 0.2 | 0.0 | 0.0 | 0.0 | 6.0 | 0.0 | 1.0 | 4.2 | 11.4 | 5.2 |
| | | | | 47.0 | 0.3 | 0.0 | 0.0 | 0.0 | 5.9 | 0.0 | 1.0 | 3.9 | 11.2 | 4.9 |
| Oil110 | | | Change | 2.6 | −0.1 | 0.0 | 0.0 | 0.0 | −1.8 | 0.0 | −0.3 | −0.1 | −2.5 | −0.5 |
| Oil110 | | | % Change | 6 | −36 | −15 | 3 | −75 | −23 | 2 | −24 | −4 | −18 | −9 |

1. A transgenic soybean seed comprising a recombinant DNA construct, the recombinant DNA construct comprising at least one polynucleotide encoding a polypeptide selected from the group consisting of (i) a DGAT polypeptide, (ii) an ODP1 polypeptide, (iii) a Lec1 polypeptide, and (iv) a combination thereof, the polynucleotide being linked to at least one regulatory sequence, and wherein the transgenic soybean seed comprises one or more of (i) a first construct down regulating GAS activity, and (ii) a second construct down regulating a fad 3 activity, a fad2 activity, or a fat2B activity, wherein the first construct and the second construct are on the same construct or are on different constructs as the recombinant DNA construct, and wherein the transgenic soybean seed has an at least 10% percent increase in total fatty acid and an at least 1% percent increase in protein, when compared to a control null segregant seed.

2. The transgenic soybean seed of claim 1, wherein the seed exhibits a percent decrease of at least one fatty acid selected from the group consisting of palmitic acid, linoleic acid and linolenic acid, when compared to a control null segregant seed.

3. The transgenic soybean seed of claim 1, wherein the seed exhibits an at least 25% increase of oleic acid when compared to a control null segregant seed.

4. The transgenic seed of claim 1, wherein the seed further exhibits an at least 60% decrease of raffinose saccharides when compared null segregant seed.

5. The transgenic seed of claim 1, wherein the seed exhibits a percent decrease in saturated fatty acids when compared to a null segregant seed.

6. The transgenic soybean seed of claim 1 wherein the at least one regulatory sequence is a soybean sucrose synthase promoter or a Medicago truncatula sucrose synthase promoter.

7. The transgenic soybean seed of claim 1, wherein the DGAT sequence is a DGAT1 sequence.

8. The transgenic soybean seed of claim 1, wherein the DGAT sequence is a DGAT2 sequence.

9. Meal prepared from the transgenic soybean seed of claim 1.

10-11. (canceled)

12. A method for increasing the total fatty acids and protein in a transgenic seed, the method comprising transforming at least one regenerable soybean cell with at least one recombinant construct comprising at least one polynucleotide encoding a polypeptide selected from the group consisting of (i) a DGAT polypeptide, (ii) an ODP1 polypeptide, (iii) a Lec1 polypeptide, and (iv) a combination thereof, the polynucleotide being linked to at least one regulatory sequence, wherein transgenic seed from the plant regenerated from the soybean cell comprises the recombinant DNA construct and one or more of a first construct down regulating GAS activity and a second construct down regulating a fad 3 activity, a fad2 activity, or a fat2B activity, wherein the first construct and the second construct are on the same construct or are on different constructs as the recombinant DNA construct, and wherein expression of the recombinant construct and the one or more constructs in the transgenic seed results in an at least 10% increase of total fatty acids and an at least 1% increase of protein, when compared to a control null segregant seed.

13. The method of claim 12, wherein the percent increase in protein is at least 4% when compared to the control null segregant seed.

14. The method of claim 12, wherein the transgenic seed further exhibits a percent decrease of at least 60% in total raffinose saccharides when compared to the null segregant seed.

15. The method of claim 12, wherein the transgenic seed further exhibits a percent decrease of total saturated fatty acids when compared to the null segregant seed.

16. The method of claim 12, wherein the transgenic seed further exhibits an at least 50% decrease of linolenic acid.

17. The method of claim 12, wherein the transgenic seed further exhibits an at least 20% increase of oleic acid.

18. The method of claim 12, wherein the DGAT sequence is DGAT1.

19. The method of claim 12, wherein the DGAT sequence is a Yarrowia or a soybean sequence.

20. The method of claim 12, wherein the at least one regulatory sequence is a soybean sucrose synthase promoter or Medicago truncatula sucrose synthase promoter.

21-24. (canceled)

25. A method of increasing the percentage of total fatty acid and protein of a soybean seed, the method comprising the steps of:
(a) crossing (I) a first transgenic soybean plant comprising a recombinant construct comprising at least one polynucleotide encoding a polypeptide selected from the group consisting of (i) a DGAT polypeptide, (ii) an ODP1 polypeptide, (iii) a Lec1 polypeptide, and (iv) a combination thereof, the polynucleotide being linked to at least one regulatory sequence, with (II) a second transgenic soybean plant comprising a second construct down regulating a fad2 activity; and
(b) selecting a third transgenic plant from the cross of step (a), wherein seed of the third transgenic plant comprises the polynucleotide and the second construct and wherein expression of the polypeptide and the second construct results in a percent increase in protein in the transgenic soybean seed, when compared to the percent increase of a control null segregant seed.

26. The third transgenic plant obtained from the method of claim 25.

27-29. (canceled)
